(19) United States
(12) Patent Application Publication
(10) Pub. No.: US 2011/0098309 A1
(43) Pub. Date: Apr. 28, 2011

(54) METHODS OF INHIBITING THE FORMATION OF AMYLOID-BETA DIFFUSABLE LIGANDS USING ACYLHYDRAZIDE COMPOUNDS

(75) Inventors: Gary Charles Look, Santa Clara, CA (US); Lauri Schultz, Batavia, IL (US); Alexandre Mikhaylovich Polozov, Naperville, IL (US); Nikhil Bhagat, Aurora, IL (US); Jian Wang, San Diego, CA (US); David E. Zembower, La Grange, IL (US); William F. Goure, Livermore, CA (US); Todd Pray, San Mateo, CA (US); Grant A. Krafft, Glenview, IL (US)

(73) Assignee: ACUMEN PHARMACEUTICALS, INC., Livermore, CA (US)

(21) Appl. No.: 12/668,174

(22) PCT Filed: Jul. 11, 2008
(86) PCT No.: PCT/US08/69878

§ 371 Date: Nov. 22, 2010

Related U.S. Application Data
(63) Continuation-in-part of application No. 11/777,264, filed on Jul. 12, 2007.
Application No. 11/777,264 is a continuation-in-part of application No. 11/777,266, filed on Jul. 12, 2007.
(60) Provisional application No. 60/950,724, filed on Jul. 19, 2007.
(60) Provisional application No. 60/950,810, filed on Jul. 19, 2007.

(51) Int. Cl. A61K 31/4965; C12N 5/071; A61K 31/16; A61K 31/343; A61K 31/341; A61K 31/381; A61K 31/40; C07C 233/00; C07D 333/56; C07D 333/22; C07D 207/30; C07D 241/04; C07D 307/02; A61P 25/28

(52) U.S. Cl. 514/255.01; 435/325; 514/614; 514/448; 514/461; 514/443; 514/408; 564/123; 549/49; 549/70; 548/563; 544/391; 549/483

(57) ABSTRACT

Disclosed are methods of inhibiting, regulating, and/or modulating the formation of soluble, globular, non-fibrillar, neurotoxic amyloid β1-42 oligomers from amyloid β1-42 monomers using acylhydrazide compounds. Also disclosed are methods of treating a patient suffering from diseases associated with the formation of soluble, globular, non-fibrillar, neurotoxic amyloid β1-42 oligomers using acylhydrazide compounds.

### CROSS REFERENCE TO RELATED APPLICATIONS

This application claims priority to U.S. application Ser. Nos. 11/777,264 and 11/777,266, both filed on Jul. 12, 2007 and claims the benefit under 35 U.S.C. §119(e) of U.S. Provisional Ser. Nos. 60/950,724 and 60/950,810, both filed on Jul. 19, 2007, all of which are incorporated by reference in their entirety.

### BACKGROUND OF THE INVENTION

1. Field of the Invention

This invention relates to methods of inhibiting, regulating, and/or modulating the formation of soluble, globular, non-fibrillar, neurotoxic amyloid β₁₋₄₂ oligomers from amyloid β₁₋₄₂ monomers using acylhydrazide compounds. This invention also relates to methods of treating a patient suffering from diseases associated with the formation of soluble, globular, non-fibrillar, neurotoxic amyloid β₁₋₄₂ oligomers by administering acylhydrazide compounds to the patients.

2. State of the Art

Alzheimer's disease (AD) is a fatal progressive dementia that has no cure at present. Although the molecular basis of the disease is not established, considerable evidence now implicates neurotoxins derived from amyloid beta (Aβ) peptides and in particular the 42-amino acid amyloid beta peptide (Aβ₁₋₄₂). Aβ is an amphipathic peptide, the abundance of which is increased by gene mutations and risk factors linked to AD. Fibrils formed from Aβ constitute the cores of amyloid senile plaques, which are hallmarks of AD brain. Analogous fibrils generated in vitro are lethal to cultured brain neurons. These findings provided the central rationale for the original amyloid cascade hypothesis, a theory in which memory loss was proposed to be the consequence of neuron death caused by fibrillar Aβ (Hardy and Higgins (1992) Science 256:184-185).

Despite its strong experimental support and intuitive appeal, the original amyloid cascade hypothesis has proven inconsistent with key observations, including the poor correlation between dementia and amyloid senile plaque burden (Katzman (1988) Ann. Neurol. 23(2):138-144). Using a transgenic mouse model of AD, two surprising findings were obtained when the mice were treated with monoclonal antibodies against Aβ: (1) vaccinated mice showed reversal of memory loss, with recovery evident in 24 hours; and (2) cognitive benefits of vaccination accrued despite no change in senile plaque levels (Dodart et al. (2002) Nat. Neurosci 5:452-457; Kotilinek et al. (2002) J. Neurosci. 22:6331-6335). Such findings are not consistent with a mechanism for memory loss dependent on neuron death caused by amyloid fibrils.

Salient flaws in the original amyloid cascade hypothesis have been eliminated by an updated amyloid cascade hypothesis that incorporates a role for additional neurologically active molecules formed by Aβ self-assembly. These molecules are amyloid β-derived diffusible ligands (ADDLs), which assemble from Aβ₁₋₄₂ at low concentrations (Lambert et al. (1998) Proc. Natl. Acad. Sci. USA 95:6448-6453). Essentially the missing links in the original amyloid cascade hypothesis, ADDLs rapidly inhibit long term potentiation (Lambert et al. (1998) Proc. Natl. Acad. Sci. USA 95:6448-6453; Walsh et al. (2002) Nature 416: 535-539; Wang et al. (2002) Brain Res. 924:133-140), a classic experimental paradigm for memory and synaptic plasticity. In the updated Aβ cascade hypothesis memory loss stems from synapse failure, prior to neuron death, with failure being caused by ADDLs, not fibrils (Hardy and Selkoe (2002) Science 297:353-356). ADDLs occur in brain tissue and are strikingly elevated in AD brain tissue compared to age matched controls (Kayed et al. (2002) Science 300:486-489; Gong et al. (2003) Proc. Natl. Acad. Sci. USA 100:10417-10422) and in AD transgenic mice models (Kotilinek et al. (2002) J. Neurosci. 22:6331-6335; Chang et al. (2003) J. Mol. Neurosci. 20:305-313).

A simplistic mechanistic approach to this theory can be illustrated as follows:
amyloid senile plaque [symbol] Monomeric AB₁₋₄₂ [symbol] ADDLs
   where formation of ADDLs is a separate pathway from formation of amyloid senile plaque both of which are in equilibrium with monomeric Aβ₁₋₄₂.

Further experiments have shown important neurological properties of ADDLs. ADDLs were shown to have selective toxicity to hippocampal CA1 neurons compared with CA3 neurons, and the complete absence of toxicity towards cerebellar neurons (Kim et al. (2003) FASEB J. 17:118-120). Ventricular injection of Aβ₁₋₄₂ oligomers into wild-type rats resulted in rapid, compromised behavioral models with complete recovery occurring within 24 hours (Cleary et al. (2005) Nat. Neurosci. 8:79-84) and these deficits are attributed to higher order oligomers, specifically 12-mer oligomers (Lesne et al. (2006) Nature 440:352-357). ADDL binding to neurons occurs with high specificity and is localized to post-synaptic receptors on a subset of hippocampal neurons (Lacor et al. (2004) J. Neurosci. 24:10191-10200). This triggers the rapid and persistent up-regulation of the immediate early gene product arc, translation of which is activity dependent at polyribosomes localized to subsets of dendritic spines (Steward et al. (1998) Neuron 21:741-751; Guzowski et al. (2000) J. Neurosci. 20:3993-4001). More recently, ADDLs have been implicated as upstream activators of tau phosphorylation and have been shown to interfere with animal behavior at femtomolar levels (Matsubara et al. (2004) Neurobiol. Aging 25:833-841).

The reversibility of memory loss in mouse models, coupled with the neurological properties of ADDLs and their presence in an AD brain, provides strong support for the hypothesis that AD is a disease of ADDL-induced synaptic failure (Lambert et al. (1998) Proc. Natl. Acad. Sci. USA 95:6448-6453; Klein et al. (2001) Trends Neurosci. 24:219-220; Selkoe (2002) Science 298:789-791).

The use of antibodies specific to ADDLs is a powerful way to modulate the equilibrium between monomeric Aβ₁₋₄₂ and ADDLs thereby providing treatment for disease conditions mediated by ADDLs. However, antibody delivery is typically limited to injectable solutions which pose patient compliance issues as well as the presence of an attending clinician. Small molecules that modulate this equilibrium, deliverable by non-injectable means such as oral delivery, transdermal delivery, pulmonary delivery, nasal delivery, etc. would be particularly beneficial.

A number of small molecules developed originally as amyloid fibril blockers are purported to possess Aβ oligomer assembly blocking properties. Some of these compounds include Alzhmed™ (Gervais (2004) Neurobiol. Aging 25:S11-12), Clioquinol (Ritchie et al. (2003) Arch. Neurol. 60:1685-1691), substituted β-cyclodextrins (Yu et al. (2002) J. Mol. Neurosci. 19:51-55), trehalose (Lui (2005) Neurobiol. Disease 20:74-81), simple amino, carbonyl, and nitro substituted phenols (De Felice et al. (2001) FASEB J. March 20; De Felice et al. (2004) FASEB J. 18:1366-1372), Curcumin (Yang et al. (2005) J. Biol. Chem. 280(7):5892-5901), cyclohexanehexyl analogs (McLaurin et al. (2006) Nature Med. 12:801-808), spirosterols (Lecanu et al. (2004) Steroids 69:1-16) and tricyclic pyrones (Maeqawa et al. (2006) J. Neurochem. 98:57-67). Two of these compounds, Alzhemed™ and Clioquinol, have progressed into clinical trials.

Alzhemed™ (3-amino-1-propanesulfonic acid), a so-called “GAG mimetic,” is proposed to reduce soluble and insoluble amyloid levels by binding to Aβ monomer, although no experimental details have appeared to confirm the proposed mode of action. Alzhemed™ has recently completed a 20 month open-label extension of a Phase II trial, and there are reports of slowed cognitive decline in some patients with mild AD, however, no efficacy was observed during the blinded phase of the study (Gervais (2004) Neurobiol. Aging 25:S11-12).

The second compound in a phase II clinical trial, Clioquinol, was shown to stabilize the patients' cognitive ability compared to untreated patients and showed lower Aβ₁₋₄₂ levels in their plasma (Ritchie et al. (2003) Arch. Neurol. 60:1685-1691). However, a toxic impurity (a di-iodo form of Clioquinol) made during production has resulted in the study being halted and Clioquinol being replaced with an analog termed PBT2 (Blennow et al. (2006) Lancet 368:387-403).

Lastly, an unidentified compound or compounds from an extract of ginko biloba leaves was reported to lower the levels of Aβ₁₋₄₂ trimers and tetramers and increase the levels of high molecular weight polymers in a dose dependent manner (Yao et al. (2001) Brain Res. 889:181-190). Dose dependent protection against Aβ oligomer induced toxicity to PC-12 cells was also reported.

Of the compounds reported to block Aβ assembly or bind to Aβ₁₋₄₂ monomer, few appear to have high therapeutic potential. Given its very simple structure and hydrophilic properties, it is highly unlikely that Alzhemed™ has high and selective affinity for Aβ₁₋₄₂ monomer. Any effect that Alzhemed™ has on Aβ aggregation or disaggregation is likely attributable to its interaction with ionic residues near the N-terminus of Aβ₁₋₄₂. The cyclodextrins do not have either lead-like or drug-like properties that would recommend them for development (Oprea et al. (2001) J. Chem. Inf. Comput. Sci. 41:1308-1315; Vieth et al. (2004) J. Med. Chem. 47:224-232), and the phenols of De Felice contain aldehyde and nitro functionalities that are often considered reactive and excluded from pharmaceutical screening libraries (Walters and Namchuk (2003) Nat. Rev. 2:259-266). A number of molecules containing the phenol functionality have been reported as “frequent hitters” in screening libraries (Roche et al. (2002) J. Med. Chem. 45:137-142). Thus, further evaluation of the activity and selectivity of the phenols of De Felice is needed to confirm that these compounds are valid hits. Some compounds with a steroidal backbone have been reported to be promiscuous inhibitors due to an unexpected self aggregation process (McGovern et al. (2002) J. Med. Chem. 45:1712-1722), which may explain the ambiguous spirosterol results. Finally, the active ingredient in the ginko biloba extract is unknown. Thus, most of the purported Aβ assembly blockers would not be considered compounds for therapeutic development.

Notwithstanding these putative results and as noted above, binding assays indicate that these compounds are, at best, moderate antagonists to ADDL formation. Accordingly, it would be particularly beneficial to provide for small molecules which provide enhanced inhibition, regulation, and/or modulation of ADDL formation.

### SUMMARY OF THE INVENTION

This invention is directed to the discovery that soluble, globular, non-fibrillar, neurotoxic Aβ₁₋₄₂ (ADDL) formation can be antagonized by certain compounds. It is contemplated that by antagonizing (or inhibiting, modulating, or regulating) ADDL formation, these compounds may be used to treat patients suffering from diseases mediated, at least in part by, ADDL formation. It is further contemplated that these compounds may also be used to inhibit, modulate or regulate neuronal dysfunction or neurotoxicity that is caused by ADDLs.

In one embodiment of the invention, the methods employ compounds of the formula:

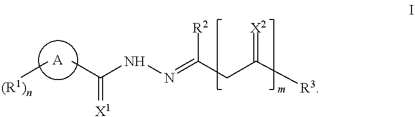

wherein:

A is a 5-10 membered heteroaryl ring having 1 to 3 heteroatoms or an aryl ring;

X¹ and X² are independently selected from the group consisting of oxygen, sulfur or N—OR⁴;

R¹ is selected from the group consisting of hydroxy, halo, nitro, C₁₋₆ alkyl, C₁₋₆ haloalkyl, C₁₋₆ alkoxy, —N(R⁵)(R⁶), C₃₋₁₀ cycloalkyl, aryl, heteroaryl, heterocyclic, wherein the aryl, heteroaryl, and heterocyclic group is optionally substituted with 1-3 R⁹ groups;

R² is selected from the group consisting of hydrogen, C₁₋₆ alkyl, and C₁₋₆ haloalkyl;

R³ is selected from the group consisting of C₁₋₆ alkyl, C₁₋₆ alkoxy, —N(R⁵)(R⁶), and R⁸.

R⁴ is selected from the group consisting of hydrogen and C₁₋₆ alkyl;

each R⁵ is independently selected from the group consisting of hydrogen, C₁₋₆ alkyl, —C(═O)—C₁₋₆ alkyl, and —SO₂(R⁷);

each R⁶ is independently selected from the group consisting of hydrogen and C₁₋₆ alkyl;

R⁷ is selected from the group consisting of hydrogen, C₁₋₆ alkyl, and aryl optionally substituted with 1 to 3 of C₁₋₄ alkyl or halo;

R⁸ is selected from the group consisting of aryl, biaryl, biaryl, heteroaryl, and heterocyclic, wherein each R⁸ is optionally substituted with 1-4 R⁹ groups;

each R⁹ is independently selected from the group consisting of hydroxy, halo, nitro, C₁₋₆ alkyl, C₂₋₆ alkenyl, C₁₋₆ alkoxy, C₁₋₆ haloalkoxy, C₃₋₁₀ cycloalkyl, aralkyl, aryl, —N(R⁵)(R⁶), carboxyl, carboxyl ester, and heterocyclic;

n is 0, 1, 2, or 3; and

m is 0 or 1;

or a pharmaceutically acceptable salt, stereoisomer, tautomer, or prodrug thereof.

The invention also contemplates using compounds of formula II or III:

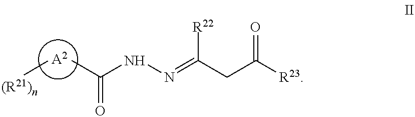

wherein:

A² is a 5-10 membered heteroaryl ring having 1 to 3 heteroatoms or an aryl ring;

R²¹ is selected from the group consisting of hydroxy, halo, nitro, C₁₋₆ alkyl, C₁₋₆ haloalkyl, —N—S(O)₂—R²⁴, and aryl;

R²² is selected from the group consisting of hydrogen, C₁₋₆ alkyl, and C₁₋₆ haloalkyl;

R²³ is selected from the group consisting of C₁₋₆ alkyl, amino, and R²⁵;

R²⁴ is selected from the group consisting of C₁₋₆ alkyl, and aryl optionally substituted with halo or C₁₋₆ alkyl,

R²⁵ is selected from the group consisting of aryl, heteroaryl, and heterocyclic, all of which may be optionally substituted with 1-3 R²⁶ groups;

each R²⁶ is independently selected from the group consisting of hydroxy, halo, C₁₋₆ alkyl, aralkyl, and aryl;

n is 0, 1, 2, or 3;

or a pharmaceutically acceptable salt, stereoisomer, tautomer, or prodrug thereof.

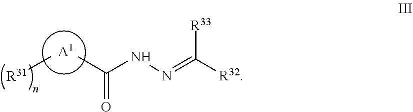

wherein:

A¹ is a 5-10 membered heteroaryl ring having 1 to 3 heteroatoms or an aryl ring;

R³¹ is selected from the group consisting of hydroxy, halo, nitro, C₁₋₆ alkyl, C₁₋₆ alkoxy, C₃₋₁₀ cycloalkyl, aryl, heteroaryl optionally substituted with 1-3 C₁₋₆ alkyl, amino, —N—SO₂—R³⁴, and —N—C(═O)—R³⁴;

R³² is selected from the group consisting of hydrogen, C₁₋₆ alkyl, and C₁₋₆ haloalkyl;

R³³ is selected from the group consisting of aryl, biaryl, heteroaryl, and heterocyclic, wherein each R³³ is optionally substituted with 1-4 R³⁵ groups;

R³⁴ is selected from the group consisting of C₁₋₆ alkyl and aryl optionally substituted with halo;

R³⁵ is selected from the group consisting of hydroxy, nitro, halo, C₁₋₆ alkyl, C₂₋₆ alkenyl, C₁₋₆ alkoxy, C₁₋₆ haloalkoxy, C₃₋₁₀ cycloalkyl, halo, amino, alkylamino, dialkylamino, aminoacyl, aryl-alkylene, carboxyl, carboxyl ester, and heterocyclic; and

n is 0, 1, 2, or 3;

or a pharmaceutically acceptable salt, stereoisomer, tautomer, or prodrug thereof.

Accordingly, in one of its method aspects, this invention is directed to a method for antagonizing neurotoxic ADDL formation from monomeric Aβ₁₋₄₂ by contacting monomeric Aβ₁₋₄₂ with an effective amount of a compound of formula I, II, or III.

In yet another embodiment, the invention is directed to a method of inhibiting, regulating and/or modulating the ADDL-induced neuronal dysfunction and/or neurotoxicity in a neuronal cell by inhibiting the formation of ADDLs. The method comprises contacting Aβ₁₋₄₂ monomers which may be in the presence of neuronal cells with an effective amount of a compound of formula I, II, or III.

In another embodiment, the invention is directed to a method of inhibiting, regulating and/or modulating amyloid-β oligomer formation or the activity of such oligomers in a patient suffering from or at risk from suffering from a disease associated with the formation of Aβ₁₋₄₂ oligomers. The method comprises administering to the patient a therapeutically effective amount of a compound of formula I, II, or III.

In yet another embodiment, the invention is directed to a method for treating a patient suffering from or at risk of suffering from an ADDL-related disease selected from the group consisting of Alzheimer's disease, Down's Syndrome, stroke, mild cognitive impairment, focal ischemia associated dementia, and neuronal degeneration. The method comprises administering to said patient a therapeutically effective amount of a compound of formula I, II, or III.

In another embodiment, the present invention relates to a composition for use in the treatment of a patient suffering from or at risk of suffering from a disease selected from the group consisting of Alzheimer's disease, Down's Syndrome, stroke, mild cognitive impairment, focal ischemia associated dementia, and neuronal degeneration wherein the composition comprises a therapeutically effective amount of a compound of formula I, II, or III.

One embodiment of the invention is directed to a method of enhancing cognitive function in a patient who has diminished cognitive function due to ADDL neurotoxicity. The method comprises administering to the patient a therapeutically effective amount of a compound of formula I, II, or III.

In some embodiments of the invention, the diminished cognitive function in a patient is due to the patient suffering from or at risk of suffering from a disease associated with the formation of and/or activity of ADDLs. In other embodiments, the disease is selected from the group consisting of Alzheimer's disease, Down's Syndrome, stroke and mild cognitive impairment.

In other embodiments of the invention, the diminished cognitive function in a patient is due to the patient suffering from or at risk of suffering from a disease associated with insoluble amyloid fibrils, senile plaques, and/or tangles. Alternatively, the diminished cognitive function in a patient is due to the patient suffering from or at risk from suffering from a disease associated with over-expression of Aβ₁₋₄₂ protein. In some embodiments, the disease is selected from the group consisting of focal ischemia associated dementia and neuronal degeneration.

In another embodiment, the invention is directed to a method of inhibiting, regulating and or modulating the binding of neurotoxic ADDLs to spines and/or synapses of a neuronal cell. The method comprises contacting said neuronal cell with an effective amount of a compound of formula I, II, or III.

In yet another embodiment, the invention is directed to a method of inhibiting, regulating and/or modulating the long term potentiation of neuronal cells. The method comprises contacting said cells with an effective amount of a compound of formula I, II, or III.

In another embodiment, the invention is directed to a method of treating a patient suffering from diminished cognitive function due to the patient suffering from or at risk of suffering from a disease selected from the group consisting of Alzheimer's disease, Down's Syndrome, stroke, mild cognitive impairment, focal ischemia associated dementia and neuronal degeneration. The method comprises administering to said patient a therapeutically effective amount of a compound of formula I, II, or III.

In one embodiment of the invention, the compound is administered in an amount of from about 0.05 milligrams to 1000 milligrams, one or more times per day. In another embodiment of the invention, the compound is administered in a pharmaceutical composition, further comprising a pharmaceutically acceptable excipient.

In one embodiment of the application, the compounds of the invention have an IC₅₀ of about 50 μM or less when tested in the FRET assay. It is contemplated that compounds of this invention have an IC₅₀ of about 50 μM or less in an assay that tests for formation of ADDLs. In one embodiment, this assay is Example 16 described below. In another embodiment of the invention, the compounds have an IC₅₀ of 25 μM or less when tested in the FRET assay. In another embodiment of the invention, the compounds have an IC₅₀ of 10 μM or less. In yet another embodiment of the invention, the compounds have an IC₅₀ of 5 μM or less.

Also included within in the scope of the invention is a compound selected from the group consisting of:
2-hydroxy-N′-(1,1,1-trifluoro-4-(furan-2-yl)-4-oxobutan-2-ylidene)benzohydrazide;
N′-(4-(4-benzylpiperazin-1-yl)-1,1,1-trifluoro-4-oxobutan-2-ylidene)-2-hydroxybenzohydrazide;
3-chloro-6-fluoro-N′-(4-(furan-2-yl)-4-oxobutan-2-ylidene)benzo[b]thiophene-2-carbohydrazide;
N-(2-(2-(1,1,1-trifluoro-4-oxo-4-(thiophen-2-yl)butan-2-ylidene)hydrazinecarbonyl)phenyl)methanesulfonamide;
2-hydroxy-N′-(1,1,1-trifluoro-4-(4-methylpiperazin-1-yl)-4-oxobutan-2-ylidene)benzohydrazide;
2-hydroxy-N′-(1,1,1-trifluoro-5,5-dimethyl-4-oxohexan-2-ylidene)benzohydrazide;
N′-(4-(benzo[b]thiophen-2-yl)-1,1,1-trifluoro-4-oxobutan-2-ylidene)-2-hydroxybenzohydrazide;
2-hydroxy-N′-(1,1,1-trifluoro-4-(1-methyl-1H-pyrazol-5-yl)-4-oxobutan-2-ylidene)benzohydrazide;
3-chloro-6-fluoro-N′-(1,1,1-trifluoro-4-(5-methylthiophen-2-yl)-4-oxobutan-2-ylidene)benzo[b]thiophene-2-carbohydrazide;
3-chloro-6-fluoro-N′-(1,1,1-trifluoro-5,5-dimethyl-4-oxohexan-2-ylidene)benzo[b]thiophene-2-carbohydrazide;
2-hydroxy-N′-(1,1,1-trifluoro-4-(5-methylthiophen-2-yl)-4-oxobutan-2-ylidene)benzohydrazide;
3-chloro-N′-(1,1,1-trifluoro-4-oxo-4-(thiophen-2-yl)butan-2-ylidene)benzo[b]thiophene-2-carbohydrazide;
3-chloro-N′-(1,1,1-trifluoro-4-(furan-2-yl)-4-oxobutan-2-ylidene)benzo[b]thiophene-2-carbohydrazide;
4-chloro-N-(2-(2-(1,1,1-trifluoro-4-oxo-4-(thiophen-2-yl)butan-2-ylidene)hydrazinecarbonyl)phenyl)benzenesulfonamide;
N′-(4-(benzo[b]thiophen-2-yl)-1,1,1-trifluoro-4-oxobutan-2-ylidene)-3-chloro-6-fluorobenzo[b]thiophene-2-carbohydrazide;
2-hydroxy-N′-(1,1,1-trifluoro-4-morpholino-4-oxobutan-2-ylidene)benzohydrazide;
N′-(4-(5-chlorothiophen-2-yl)-1,1,1-trifluoro-4-oxobutan-2-ylidene)-2-hydroxybenzohydrazide;
5-chloro-2-hydroxy-N′-(1,1,1-trifluoro-4-oxo-4-(thiophen-2-yl)butan-2-ylidene)benzohydrazide;
3-chloro-4-methyl-N′-(1,1,1-trifluoro-4-oxo-4-(thiophen-2-yl)butan-2-ylidene)thiophene-2-carbohydrazide;
N′-(1,1,1-trifluoro-4-oxo-4-(thiophen-2-yl)butan-2-ylidene)-1H-indole-7-carbohydrazide;
3-chloro-N′-(4-(5-chlorothiophen-2-yl)-1,1,1-trifluoro-4-oxobutan-2-ylidene)-6-fluorobenzo[b]thiophene-2-carbohydrazide;
3-chloro-6-fluoro-N′-(1,1,1-trifluoro-4-oxo-4-(thiophen-2-yl)butan-2-ylidene)benzo[b]thiophene-2-carbohydrazide;
2-hydroxy-3-methyl-N′-(1,1,1-trifluoro-4-oxo-4-(thiophen-2-yl)butan-2-ylidene)benzohydrazide;
4-nitro-N′-(1,1,1-trifluoro-4-oxo-4-(thiophen-2-yl)butan-2-ylidene)benzohydrazide;
N′-(1,1,1-trifluoro-4-oxo-4-(thiophen-2-yl)butan-2-ylidene)benzo[b]thiophene-2-carbohydrazide;
2-hydroxy-N′-(4-oxo-4-(4-phenylpiperidin-1-yl)butan-2-ylidene)benzohydrazide;
3-chloro-N′-(1,1,1-trifluoro-4-oxo-4-(thiophen-2-yl)butan-2-ylidene)-1H-indole-2-carbohydrazide;
3-chloro-6-fluoro-N′-(1,1,1-trifluoro-4-(furan-2-yl)-4-oxobutan-2-ylidene)benzo[b]thiophene-2-carbohydrazide;
3-(2-(2-hydroxybenzoyl)hydrazono)butanamide;
1-phenyl-N′-(1,1,1-trifluoro-4-oxo-4-(thiophen-2-yl)butan-2-ylidene)-5-(trifluoromethyl)-1H-pyrazole-4-carbohydrazide;
2,6-difluoro-N′-(1,1,1-trifluoro-4-oxo-4-(thiophen-2-yl)butan-2-ylidene)benzohydrazide;
2-hydroxy-N′-(2-hydroxy-5-methoxybenzylidene)-4-(1H-pyrrol-1-yl)benzohydrazide;
1-hydroxy-N′-(2-hydroxy-5-methoxybenzylidene)-2-naphthohydrazide;
N′-(3-chloro-5-fluoro-2-hydroxybenzylidene)-2-hydroxybenzohydrazide;
N′-(2,6-dihydroxybenzylidene)-2-hydroxybenzohydrazide;
3-chloro-N′-(2-hydroxy-5-methoxybenzylidene)-1H-indole-2-carbohydrazide;
N′-(2-bromo-6-hydroxybenzylidene)-2-hydroxybenzohydrazide;
N′-(3-chloro-5-cyclohexyl-2-hydroxybenzylidene)-2-hydroxybenzohydrazide;
N′-(5-tert-butyl-2-hydroxybenzylidene)-2-hydroxybenzohydrazide;
2-hydroxy-N′-((4-hydroxy-3′-methoxybiphenyl-3-yl)methylene)benzohydrazide;
4-chloro-2-hydroxy-N′-(2-hydroxy-5-methoxybenzylidene)benzohydrazide;
N′-(2-hydroxy-5-methoxybenzylidene)-2-oxoindoline-7-carbohydrazide;
2-hydroxy-N′-(2-hydroxy-6-methoxybenzylidene)benzohydrazide;
2-hydroxy-N′-(2-hydroxy-5-(trifluoromethoxy)benzylidene)benzohydrazide;
N′-(2-hydroxy-5-methoxybenzylidene)-1H-indole-7-carbohydrazide;
3-chloro-6-fluoro-N′-((2-hydroxynaphthalen-1-yl)methylene)benzo[b]thiophene-2-carbohydrazide;
2-hydroxy-N′-(2-hydroxy-5-methoxybenzylidene)-3-methylbenzohydrazide;
2-((2-(2-hydroxybenzoyl)hydrazono)methyl)benzamide;
N′-(2-amino-5-chlorobenzylidene)-2-hydroxybenzohydrazide;
2-hydroxy-N′-(2-hydroxy-5-methoxybenzylidene)-1-naphthohydrazide;
4-fluoro-2-hydroxy-N′-(2-hydroxy-5-methoxybenzylidene)benzohydrazide;
3-chloro-N′-(2-hydroxy-5-methoxybenzylidene)-4-methylthiophene-2-carbohydrazide;
2-hydroxy-N′-(2-hydroxy-5-methoxybenzylidene)-5-methylbenzohydrazide;
N′-(3-fluoro-2-hydroxybenzylidene)-2-hydroxybenzohydrazide;
5-fluoro-2-hydroxy-N′-(2-hydroxy-5-methoxybenzylidene)benzohydrazide;
ethyl 6-bromo-5-hydroxy-4-(2-(2-hydroxybenzoyl)hydrazono)methyl)-2-methylbenzofuran-3-carboxylate;
3-(benzo[d][1,3]dioxol-5-yl)-N′-(2-hydroxy-5-methoxybenzylidene)-1H-pyrazole-5-carbohydrazide;
N′-(4-(diethylamino)-2-hydroxybenzylidene)-2-hydroxybenzohydrazide;
2-hydroxy-N′-(2-hydroxy-4-methoxybenzylidene)benzohydrazide;
2-hydroxy-N′-(2-hydroxy-5-methoxybenzylidene)-5-methoxybenzohydrazide;
2-hydroxy-N′-(2-hydroxy-5-methoxybenzylidene)benzohydrazide;
N′-(5-ethoxy-2-hydroxybenzylidene)-2-hydroxybenzohydrazide;
N′-(2,3-dihydroxybenzylidene)-2-hydroxybenzohydrazide;
2-hydroxy-N′-(2-hydroxy-4-morpholinobenzylidene)benzohydrazide;
2-hydroxy-N′-((3-hydroxy-5-nitrobenzofuran-2-yl)methylene)benzohydrazide;
N′-(2,4-dihydroxybenzylidene)-2-hydroxybenzohydrazide;
N′-(5-chloro-2-hydroxy-3-methoxybenzylidene)-2-hydroxybenzohydrazide;
3-chloro-N′-(5-chloro-2-hydroxybenzylidene)-4-methylthiophene-2-carbohydrazide;
2-amino-N′-(2-amino-5-chlorobenzylidene)benzohydrazide;
2-hydroxy-N′-(2-hydroxy-3-methoxybenzylidene)benzohydrazide;
2-fluoro-6-hydroxy-N′-(2-hydroxy-5-methoxybenzylidene)benzohydrazide;
2-hydroxy-N′-(2-hydroxy-3-methylbenzylidene)benzohydrazide;
N′-((2-hydroxynaphthalen-1-yl)methylene)-3-methyl-1H-pyrazole-5-carbohydrazide;
5-bromo-2-hydroxy-N′-(2-hydroxy-5-methoxybenzylidene)benzohydrazide;
2-hydroxy-N′-(2-hydroxy-5-nitrobenzylidene)benzohydrazide;
N-(2-(2-(2-hydroxy-5-methoxybenzylidene)hydrazinecarbonyl)phenyl)methanesulfonamide;
N′-(3,5-difluoro-2-hydroxybenzylidene)-2-hydroxybenzohydrazide;
N′-(1-(5-chloro-2-hydroxyphenyl)-2,2,2-trifluoroethylidene)-2-hydroxybenzohydrazide;
2-hydroxy-N′-(2-hydroxy-5-methoxybenzylidene)-5-nitrobenzohydrazide;
8-hydroxy-N′-(2-hydroxy-5-methoxybenzylidene)-1-naphthohydrazide;
N′-(3-ethoxy-2-hydroxybenzylidene)-2-hydroxybenzohydrazide;
3-(5-chlorothiophen-2-yl)-N′-(2-hydroxy-5-methoxybenzylidene)-1H-pyrazole-5-carbohydrazide;
N′-(3-bromo-5-chloro-2-hydroxybenzylidene)-2-hydroxybenzohydrazide;
N′-(3-bromo-2-hydroxy-5-methoxybenzylidene)-2-hydroxybenzohydrazide;
2-amino-N′-(2-hydroxy-5-methoxybenzylidene)benzohydrazide;
N′-(5-chloro-2-hydroxybenzylidene)-4-methyl-1,2,3-thiadiazole-5-carbohydrazide;
N-(2-(2-(2-acetamido-5-chlorobenzylidene)hydrazinecarbonyl)phenyl)acetamide; and
N-(4-chloro-2-(2-(2-hydroxybenzoyl)hydrazono)methyl)phenyl)-2,4-difluorobenzenesulfonamide;

or a pharmaceutically acceptable salt, stereoisomer, tautomer, or prodrug thereof.

### DETAILED DESCRIPTION OF THE INVENTION

### A. Methods of the Invention

Before the methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless the context clearly dictates otherwise.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety. Nothing herein is to be construed as an admission that the invention is not entitled to antedate such disclosure by virtue of prior invention.

The definitions used herein are limited to the application of small molecules as they relate to ADDL aggregation or oligomerization and diseases mediated by such.

This invention is directed to the discovery that the formation of soluble, oligomeric, globular, non-fibrillar, neurotoxic Aβ₁₋₄₂ peptides (ADDLs) can be antagonized by compounds of formula I, II, or III. Without being limited by any theory, it is believed that the administration of a therapeutically effective amount of one or more of the compounds described herein will interact with key assembly motifs within the Aβ₁₋₄₂ monomers or within critical motifs on the Aβ₁₋₄₂ oligomers. This interaction, in turn, will prevent the formation of neurotoxic ADDLs or the activity of such ligands. The disruption of the ADDLs or the activity of such ligands will protect long term potentiation of neuronal cells thereby obviating and/or reversing the neurotoxicity associated with ADDL. In addition, this interaction does not interfere with the formation of Aβ senile plaques.

The term “ADDL” is conventionally defined as amyloid beta-derived diffusable ligands which have the following characteristics: soluble, oligomeric, globular, non-fibrillar, neurotoxic Aβ₁₋₄₂ peptides (GenBank Ref. No. IZOQ_A, accessed on Nov. 21, 2007).

The compounds described herein are useful in a method for inhibiting, regulating and/or modulating assembly of ADDLs either in vitro or in vivo.

The term “soluble” means the ability for a given substance, the solute (an example in the instant invention is the Aβ₁₋₄₂ oligomer), to dissolve in a solvent. Within the context of the instant invention, soluble Aβ oligomers are capable of being fractionated by centrifugation.

The term “oligomeric” means a protein complex of a finite number of monomer subunits. In the context of the invention, oligomers are referred to as trimers, low-n-mers, dodecamers (12-mers), and large-n-multimers composed of Aβ₁₋₄₂ peptides. The term “oligomeric” does not include senile amyloid plaques.

The term “globular” means a large soluble protein complex, which is to be distinguished from fibrils and amyloid plaques. Preferably, the globular structure ranges in size from 4 nanometers (nm) to about 12 nm, preferably, from about 4.7 to about 11 nm, which can be observed upon atomic force microscope analysis (AFM) of supernatant fractions of Aβ₁₋₄₂ soluble oligomer preparations as described in U.S. Pat. No. 6,218,506.

The term “non-fibrillar” means the Aβ₁₋₄₂ peptides and oligomeric complexes that are not aligned in a morphologically distinct pattern known as amyloid protofibrils or amyloid fibrils.

As mentioned above, the compounds described herein are useful for antagonizing ADDL formation in vivo and the diseases associated with ADDL formation. As such, the terms “disease,” “disorder,” and “condition” are used inclusively and refer to any condition mediated, at least in part, by ADDLs. In the context of this invention the disease may be associated with insoluble amyloid fibrils, senile plaques, neurofibrillary tangles, and/or the over-expression of amyloid β₁₋₄₂ protein. Examples include, but are not limited to, Alzheimer's disease, Down's Syndrome, mild cognitive impairment, stroke, focal ischemia associated dementia, and neuronal degeneration. Patients amenable to treatment include individuals at risk of disease but not exhibiting symptoms, as well as patients presently exhibiting symptoms. Therefore, the compounds described herein can be administered prophylactically to the general population without the need for any assessment of the risk of the patient.

The term “amyloid fibrils” means protein aggregates sharing specific structural traits. Histopathological techniques generally identify the structures by apple-green birefringence when stained with Congo red and seen under polarized light.

The term “senile plaque” or “senile plaque formation” refers to the extracellular deposit of amyloid in the gray matter of the brain. The deposits are associated with degenerative neural structures. It is understood that senile plaque is different from and distinguished over ADDLs.

The term “tangles” means the neurofibrillary tangles formed inside of degenerating neurons by bundling of paired helical filaments, which assemble from hyperphosphorylated forms of the microtubule-associated protein know as tau.

The term “patient” refers to animals, including mammals, humans, and non-human mammals. In certain embodiments, a patient is an animal, particularly an animal selected from a mammalian species including rat, rabbit, bovine, ovine, porcine, canine, feline, murine, equine, and primate, particularly human.

The methods are especially useful for patients who have a known genetic risk of Alzheimer's disease. Such individuals include those having relatives who have been diagnosed with the disease and those whose risk is determined by analysis of genetic or biochemical markers. Genetic markers of risk for Alzheimer's disease include mutations in the APP gene, particularly mutations at position 717 and positions 670 and 671 referred to as the Hardy and Swedish mutations respectively. Other markers of risk are mutations in the presenilin genes, PS1 and PS2, and ApoE4, family history of Alzheimer's Disease, hypercholesterolemia or atherosclerosis. Individuals presently suffering from Alzheimer's disease can be recognized from characteristic dementia, as well as the presence of risk factors described above. In addition, a number of diagnostic test are available for identifying individuals who have Alzheimer's disease. These include measurement of CSF tau as described in Vandermeeren et al. (1993) J. Neurochem. 61:1828-1834; Arai et al. (1995) Ann. Neurol. 38:649-652; and Jansen et al. (1995) Neurosci. Lett. 186:189-191 and Aβ₁₋₄₂ levels as described in Andreasen et al. (1999) Arch. Neurol. 56:673-680; Vanderstichele et al., “Development of a specific diagnostic test for measurement of β-amyloid1-42 in CSF”, Progress in Alzheimer's and Parkinson's diseases, Fisher et al. (eds), New York, Plenum, pgs. 773-778; and Hulstaert et al. (1999) Neurology 52:1555-1562. Individuals suffering from Alzheimer's disease can also be diagnosed by NINCDS-ADRDA (National Institute of Neurological and Communicative Diseases and Stroke/Alzheimer's Disease and Related Disorders Association) criteria as described in Hogervorst et al. “Diagnosing dementia: Interrater Reliability Assessment and Accuracy of the NINCDS/ADRDA Criteria versus CERAD Histopathological Criteria for Alzheimer's Disease” University of Oxford, Oxford Project to Investigate Memory and Aging (OPTIMA), Oxford, UK; and McKhann et al. (1984) Neurology 34(7):939-944.

In asymptomatic patients, treatment can begin at any age (e.g., 10, 20, 30 years of age). Usually, however, it is not necessary to begin treatment until a patient reaches about 40, 50, 60, or 70 years of age. Treatment typically entails multiple dosages over a period of time. Treatment can be monitored by assaying for the presence of ADDLs over time.

As mentioned above, the methods described herein are useful for treating patients. As used herein, the terms “Treating” or “treatment” of a disease includes: (1) preventing the disease, i.e., causing the clinical symptoms of the disease not to develop in a patient that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “suffering” as it related to the term “treatment” refers to a patient or individual who has been diagnosed with or is predisposed to a disease. A patient may also be referred to being “at risk of suffering” from a disease. This patient has not yet developed characteristic disease pathology, however are known to be predisposed to the disease due to family history, being genetically predisposed to developing the disease, or diagnosed with a disease or disorder that predisposes them to developing the disease to be treated.

In addition to Alzheimer's disease, several other disease are known to be associated with Aβ₁₋₄₂ formation including, but are not limited to, Down's Syndrome, stroke and mild cognitive impairment. It is conceivable that similar to Alzheimer's disease, treatment of patients suffering from or at risk of suffering from these diseases is possible due to the parallel mechanisms of the diseases.

Similarly, over-expression of Aβ₁₋₄₂ is associated with focal ischemia associated dementia and neuronal degeneration. Over-expression of Aβ₁₋₄₂ is believed to result in accumulation of ADDLs, thereby inducing neurotoxicity. Treating a patient suffering from or at risk of suffering from one of these diseases by administration of one or more of the compounds described herein will ameliorate the neurotoxicity of over-expressed Aβ₁₋₄₂.

The term “neurotoxicity” refers to the toxic effect of ADDLs on neuronal cells either in vitro and/or in vivo. ADDLs bind to specific neuronal receptors triggering aberrant neuronal signaling, which compromises long term potentiation and causes memory deficits. Thus, ADDLs alter the function of the neuronal cell in such a manner that, while still viable, the neuron does not properly function. Such altered functionality is referred to herein as “neuronal dysfunction,” which is a subclass of neurotoxicity. Persistent ADDL signaling causes aberrant transcription and the progressive loss of synapses, and very long term persistent ADDL signaling and accumulated structural pathology leads to eventual neuron death and gross brain dystrophy.

In therapeutic applications, a pharmaceutical composition containing one or more compounds described herein is administered to a patient suspected of, or already suffering from such a disease associated with the accumulation of ADDLs, wherein said compounds are administered in an amount sufficient to treat, or at least partially treat, the symptoms of the disease (biochemical, histological and/or behavioral), including its complication and intermediate pathological phenotypes in development of the disease. In prophylactic applications, a pharmaceutical composition containing one or more compounds described herein is administered to a patient susceptible to, or otherwise at risk of, a disease associated with the accumulation of ADDLs, wherein said compounds are administered in an amount sufficient to eliminate or reduce the risk, lessen the severity, or delay the outset of the disease. This includes biochemical, histological and/or behavioral symptoms of the disease, its complications and intermediate pathological phenotypes presenting during development of the disease.

In some methods, administration of the compound reduces or eliminates mild cognitive impairment in patients that have not yet developed characteristic Alzheimer's pathology. In particular embodiments, a therapeutically effective amount intends to indicate the amount of one or more compounds described herein administered or delivered to the patient which is most likely to result in the desired response to treatment.

The invention is directed to enhancing cognitive function in a patient who has diminished function. The term “cognitive function” refers to the intellectual process by which one becomes aware of, perceives, or comprehends ideas. Cognitive function embraces the quality of knowing, which includes all aspects of perception; recognition; conception; sensing; thinking; reasoning; remembering and imagining.

The term “diminished cognitive function” refers to memory loss, mental slowing, intellectual decline and/or amnesia. Memory loss may be characterized as the difficulty or failure for immediate or delayed recall. Mental slowing is the difficulty in processing or completing previously learned tasks in a timely manner or in processing new information quickly. Intellectual decline is defined as a loss of information, or an inability to utilize information previously possessed or utilized by a person. Amnesia is an extreme loss of cognitive ability which results in partial or total inability to recall past experiences and impaired or total loss of the ability to speak or write. Diminished cognitive function may be caused by a number of disease conditions which are more thoroughly discussed below.

Methods of assessing cognitive function include, but are not limited to, standardized instruments for example Folstein Mini-Mental State Examination; Modified Mini-Mental State Exam; Middlesex Elderly Assessment of Mental State; Short Portable Mental Status Questionnaire; Alzheimer's Disease Assessment Scale; Clock Drawing Test; Clinical Dementia Rating; Neuropsychiatric Inventory or any similarly designed test. Using the above listed tests, a skilled clinician would be able to assess the level of diminished cognitive function of a patient or enhanced cognitive function following treatment. Additionally, informal observations and interactions of individuals to a patient can also be used to assess cognitive function and include, but are not limited to, family members, friends, formal care givers such as nurses, and individuals who have previous intimate knowledge of the patient.

Mechanical measure of the neurons and neuronal tissue may also be used to assess cognitive function including, but not limited to, Computed Tomography (CT); Computed Axial Tomography (CAT); Magnetic Resonance Imaging (MRI); Functional Magnetic Resonance Imaging (fMRI); Positron Emission Tomography (PET): Single Photon Emission Computed Tomography (SPECT); Diffuse Optical Imaging (DOI); Diffuse Optical Tomography (DOT) or any similarly designed instrumentation.

An “effective amount” is an amount of one or more of the compounds described herein which treats the ADDL-mediated disease. In one embodiment, the compounds of this invention will decrease ADDL formation either in vitro or in vivo by at least 10%, 25%, 40%, 60%, 80%, 90% or 95% as compared to control.

The “therapeutically effective amount” will vary depending on the compound, the disease and its severity and the age, weight, etc., of the patient to be treated all of which is within the skill of the attending clinician to assess. It is contemplated that a therapeutically effective amount of one or more of the compounds described herein will alter ADDL formation (including inhibiting or reversing formation of ADDLs) in the patient as compared to binding of ADDLs in the absence of treatment. As such, impairment of long term potentiation and subsequent memory formation is decreased. A therapeutically effective amount is distinguishable from an amount having a biological effect (a “biologically effective amount”). A compound of the present invention may have one or more biological effects in vitro or even in vivo, such as reduction in ADDL formation to some extent. A biological effect, however, may not result in any clinically measurable therapeutically effect as described above as determined by methods within the skill of the attending clinician.

The present invention is also directed ADDL inhibition in a neuronal cell and/or neuronal tissue. A “neuronal cell” or “neuron” is a cell that transmits and processes signals in the brain or other parts of the nervous system. Additionally, a neuronal cell, as used in the invention, can be isolated from animal brain tissue and grown in tissue culture. The isolated cells can be comprised of an established neuronal cell line selected from for example, but are not limited to, MC65; HCN-2; SH-SY5Y; SK-N-AS; SK-N-FI; SK-N-DZ; H19-7/IGF-IR; QNR/D; QNR/K2; C8-D30; C8-S; C8-D1A; OLGA-PH-J/92; Daoy; RSC96; SW10; RT4-D6P2T; RN33B; PC-12; DBRTG-05MG; C8-B4; SK-N-SH; B35; R3[33-10ras3]; Neuro-2A; and HCN-1A or any genetic, chemical, and/or biochemical modified variants thereof. (Commercially available from American Type Culture Collection (ATCC)). The isolated cells can also be comprised of primary cells and/or astrocytes isolated from neuronal tissues selected from, for example, but are not limited to, the hippocampus; cerebellum; cortex; hypothalamus; mid-brain; spinal cord; striatum; frontal lobe; temporal lobe; parietal lobe; occipital lobe and any genetic, chemical, and/or biochemical modified variants thereof. The isolated, cultured animal cell can be comprised of a neural stem cell or any differentiated, genetic, chemical, and/or biochemical modified variants thereof. Additionally, a neuronal cell or neuron can be isolated and distinguished from other cell types by detecting expression of neuronal markers selected from, but not limited to, CD133, GFAP, MAP-2, MPB, Nestin, Neural tubulin, Neurofilament, Neurosphere, Noggin, O4, O1, Synaptophysin, and Tau (http://stemcells.nih.gov/info/scireport/appendixE.asp, accessed on Nov. 26, 2007).

As used herein, the term “neuronal tissue” refers to any portion of the central nervous system including, but not limited to, the brain or spinal cord. Neuronal tissue can be composed of, at least in part, neuronal cells.

### B. Compounds

The compounds useful in the methods of the invention contain one or more and any combination of the following characteristics: (1) low or sub-micromolar potency when tested in the FRET assay described herein; (2) non-aggregating; (3) little or no neuronal toxicity when administered to a patient; (4) favorable solubility in an aqueous environment; (5) chemically tractable; (6) dose dependent characteristics; (7) reversibly bind to the Aβ protein; (8) capable of amyloid β monomer binding; (9) capable of binding soluble amyloid β oligomers.

In one embodiment of the invention, the compounds useful for treating patients are suitable for oral delivery. In this embodiment, the compounds are compliant with Lipinski's rule-of-five which provides a criteria to evaluate drug likeness. The rule states that, in general, an orally active drug has: no more than 5 hydrogen bond donors (OH and NH groups); no more than 10 hydrogen bond acceptors (notably N and O); a molecular weight under 500 g/mol; and a partition coefficient log P less than 5.

### 1. Exemplary Compounds

In one embodiment, methods of the invention employ compounds of the formula:

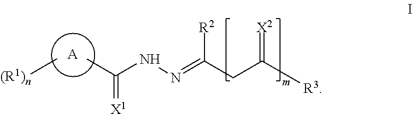

wherein:

A is a 5-10 membered heteroaryl ring having 1 to 3 heteroatoms or an aryl ring;

X¹ and X² are independently selected from the group consisting of oxygen, sulfur or N—OR⁴;

R¹ is selected from the group consisting of hydroxy, halo, nitro, C₁₋₆ alkyl, C₁₋₆ haloalkyl, C₁₋₆ alkoxy, —N(R⁵)(R⁶), C₃₋₁₀ cycloalkyl, aryl, heteroaryl, heterocyclic, wherein the aryl, heteroaryl, and heterocyclic group is optionally substituted with 1-3 R⁹ groups;

R² is selected from the group consisting of hydrogen, C₁₋₆ alkyl, and C₁₋₆ haloalkyl;

R³ is selected from the group consisting of C₁₋₆ alkyl, C₁₋₆ alkoxy, —N(R⁵)(R⁶), and R⁸;

R⁴ is selected from the group consisting of hydrogen and C₁₋₆ alkyl;

each R⁵ is independently selected from the group consisting of hydrogen, C₁₋₆ alkyl, and —SO₂(R⁷);

each R⁶ is independently selected from the group consisting of hydrogen and C₁₋₆ alkyl;

R⁷ is selected from the group consisting of hydrogen, C₁₋₆ alkyl, —C(═O)—C₁₋₆ alkyl, aryl optionally substituted with 1 to 3 of C₁₋₄ alkyl or halo;

R⁸ is selected from the group consisting of aryl, biaryl, heteroaryl, and heterocyclic, wherein each R⁸ is optionally substituted with 1-4 R⁹ groups;

each R⁹ is independently selected from the group consisting of hydroxy, halo, nitro, C₁₋₆ alkyl, C₂₋₆ alkenyl, C₁₋₆ alkoxy, C₁₋₆ haloalkoxy, C₃₋₁₀ cycloalkyl, halo, aminoacyl, acylamino, aralkyl, —N(R⁵)(R⁶), carboxyl, carboxyl ester, and heterocyclic;

n is 0, 1, 2, or 3; and

m is 0 or 1;

or a pharmaceutically acceptable salt, stereoisomer, tautomer, or prodrug thereof.

In another embodiment of the invention, the methods employ compounds of the formula:

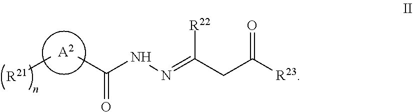

wherein:

A² is a 5-10 membered heteroaryl ring having 1 to 3 heteroatoms or an aryl ring;

R²¹ is selected from the group consisting of hydroxy, halo, nitro, C₁₋₆ alkyl, C₁₋₆ haloalkyl, —N—S(O)₂—R²⁴, and aryl;

R²² is selected from the group consisting of hydrogen, C₁₋₆ alkyl, and C₁₋₆ haloalkyl;

R²³ is selected from the group consisting of C₁₋₆ alkyl, amino, and R²⁵;

R²⁴ is selected from the group consisting of C₁₋₆ alkyl, and aryl optionally substituted with halo or C₁₋₆ alkyl,

R²⁵ is selected from the group consisting of aryl, heteroaryl, and heterocyclic, all of which may be optionally substituted with 1-3 R²⁶ groups;

each R²⁶ is independently selected from the group consisting of hydroxy, halo, C₁₋₆ alkyl, aralkyl, and aryl;

n is 0, 1, 2, or 3;

or a pharmaceutically acceptable salt, stereoisomer, tautomer, or prodrug thereof.

In yet another embodiment, methods of the invention employ a compound of the formula:

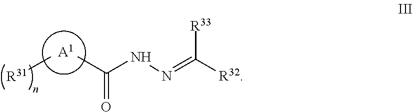

wherein:

A¹ is a 5-10 membered heteroaryl ring having 1 to 3 heteroatoms or an aryl ring;

R³¹ is selected from the group consisting of hydroxy, halo, nitro, C₁₋₆ alkyl, C₁₋₆ alkoxy, C₃₋₁₀ cycloalkyl, aryl, heteroaryl optionally substituted with 1-3 C₁₋₆ alkyl, amino, —N—SO₂—R³⁴, and —N—C(═O)—R³⁴;

R³² is selected from the group consisting of hydrogen, C₁₋₆ alkyl, and C₁₋₆ haloalkyl;

R³³ is selected from the group consisting of aryl, biaryl, heteroaryl, and heterocyclic, wherein each R³³ is optionally substituted with 1-4 R³⁵ groups;

R³⁴ is selected from the group consisting of C₁₋₆ alkyl and aryl optionally substituted with halo;

R³⁵ is selected from the group consisting of hydroxy, nitro, halo, C₁₋₆ alkyl, C₂₋₆ alkenyl, C₁₋₆ alkoxy, C₁₋₆ haloalkoxy, C₃₋₁₀ cycloalkyl, halo, amino, alkylamino, dialkylamino, aminoacyl, aryl-alkylene, carboxyl, carboxyl ester, and heterocyclic; and

n is 0, 1, 2, or 3;

or a pharmaceutically acceptable salt, stereoisomer, tautomer, or prodrug thereof.

In one embodiment, the group A is selected from the group consisting of phenyl, biphenyl, naphthyl, benzothiophenyl, thiadiazolyl, indanyl, thiophenyl, indolyl, pyrazolyl, furanyl, oxoindolinyl, pyridyl, and benzodioxoyl.

In some embodiments R¹ is selected from the group hydroxy, chloro, fluoro, bromo, iodo, methyl, methoxy, trifluoromethyl, cyclopropyl, phenyl, pyrrolyl, methylsulfonamido, 4-chlorophenylsulfonylamido, nitro, benzo[d][1,3]dioxolyl, amino, thienyl, 5-chlorothienyl, and methylcarbonylamino.

In another embodiment, the group A is optionally substituted and is selected from the group consisting of 2-(methylsulfonamido)phenyl, 1H-indan-7-yl, 1H-indol-7-yl, 1-hydroxy-naphthalen-2-yl, 1-phenyl-5-(trifluoromethyl)-1H-pyrazol-4-yl, 2-(4-chlorophenyl sulfonamido) phenyl, 2,4-dihydroxyphenyl, 2,6-difluorophenyl, 2-acetamidophenyl, 2-aminophenyl, benzothiophen-2-yl, 2-fluoro-6-hydroxyphenyl, 2-hydroxy-3-methylphenyl, 2-hydroxy-4-(1H-pyrrol-1-yl)phenyl, 2-hydroxy-4-methoxyphenyl, 2-hydroxy-5-methoxyphenyl, 2-hydroxy-5-methylphenyl, 2-hydroxy-5-nitrophenyl, 2-hydroxy-naphthalen-1-yl, 2-hydroxyphenyl, 2-methylfuran-3-yl, 2-oxoindolin-7-yl, 3-(5-chlorothiophen-2-yl)-1H-pyrazol-5-yl, 3-(benzo[d][1,3]dioxol-5-yl-1H-pyrazol-5-yl, 3-aminophenyl, 3-chloro-1H-indol-2-yl, 3-chloro-4-methyl-thiophen-2-yl, 3-chloro-6-fluorobenzo thiophene, 3-chlorobenzothiophen-2-yl, 3-cyclopropyl-1H-pyrazol-5-yl, 3-fluorophenyl, 3-hydroxy-naphthalen-2-yl, 3-methyl-1H-pyrazol-5-yl, 4-(2,5-dimethyl-1H-pyrrol-1-yl)phenyl, 4-bromophenyl, 4-chloro-2-hydroxyphenyl, 4-iodo-1-methyl-1H-pyrazol-3-yl, 4-iodophenyl, 4-methyl-1,2,3-thiadiazol-5-yl, 4-methylphenyl, 4-nitrophenyl, 5-bromo-2-hydroxyphenyl, 6-methyl-pyrid-3-yl, 8-hydroxynaphthalen-1-yl, and benzo[d][1,3]dioxol-5-yl.

In some embodiments, X¹ and X² are oxygen.

In some embodiments, R² is selected from hydrogen, methyl, or trifluoromethyl.

In some embodiments, m is 0. In other embodiments m is 1.

In some embodiments, R³ is selected from the group consisting of butyl, t-butyl, and amino.

In some embodiments, R³ is optionally substituted and is selected from the group consisting of phenyl, thienyl, naphthyl, furanyl, piperazinyl, benzothiophenyl, pyrazolyl, morpholino, and piperidinyl.

In some embodiments, R³ when a ring, is substituted with one or more groups consisting of hydroxy, chloro, fluoro, bromo, iodo, methyl, t-butyl, methoxy, ethoxy, benzyl, phenyl, cyclohexyl, trifluoromethoxy, allyl, aminocarbonyl, amino, ethoxycarbonyl, diethylamino, morpholino, nitro, 2,4-difluorophenylsulfonylamino, and methylcarbonylamino.

In other embodiments, R³ is selected from the group consisting of 5-chloro-2-(2,4-difluorophenylsulfonamido)phenyl, 1-hydroxy-napthalen-2-yl, 1-methyl-1H-pyrazol-5-yl, 2,3-dihydroxyphenyl, 2,4-dihydroxyphenyl, 2,6-dihydroxyphenyl, 2-acetamido-5-chlorophenyl, 2-amino-5-chlorophenyl, benzothiophen-2-yl, 2-bromo-6-hydroxyphenyl, 2-furanyl, 2-hydroxy naphthalen-1-yl, 4-hydroxy-3′-methoxybiphen-3-yl, 2-hydroxy-3-methoxyphenyl, 2-hydroxy-3-methylphenyl, 2-hydroxy-4-methoxyphenyl, 2-hydroxy-4-methylphenyl, 2-hydroxy-4-morpholinophenyl, 2-hydroxy-5-methoxyphenyl, 2-hydroxy-5-methylphenyl, 2-hydroxy-5-nitrophenyl, 2-hydroxy-5-trifluoromethoxyphenyl, 2-hydroxy-6-methoxy-phenyl, 2-hydroxynaphthalen-1-yl, 2-hydroxyphenyl, 3,5-dibromo-2-hydroxyphenyl, 3,5-dichloro-2-hydroxy-phenyl, 3,5-difluoro-2-hydroxyphenyl, 3-allyl-2-hydroxyphenyl, 3-bromo-2-hydroxy-5-methoxyphenyl, 3-bromo-5-chloro-2-hydroxyphenyl, 3-chloro-5-cyclohexyl-2-hydroxyphenyl, 3-chloro-5-fluoro-2-hydroxyphenyl, 3-ethoxy-2-hydroxyphenyl, 3-fluoro-2-hydroxyphenyl, 3-hydroxy-5-nitrobenzofuran-2-yl, 4-benzyl piperazin-1-yl, 4-diethylamino-2-hydroxyphenyl, 4-methyl piperazin-1-yl, 4-methylphenyl, 4-morpholino, 4-phenylpiperidin-1-yl, 5-bromo-2-hydroxy-3-iodophenyl, 5-bromo-2-hydroxyphenyl, 5-chlorothiophen-2-yl, 2-amino-5-chloro-phenyl, 5-chloro-2-hydroxy-3-methoxyphenyl, 5-chloro-2-hydroxyphenyl, 5-chlorothiophen-2-yl, 5-ethoxy-2-hydroxyphenyl, 5-methyl thiophen-2-yl, 5-tert-butyl-2-hydroxyphenyl, 6-bromo-5-hydroxy-2-(ethoxycarbonyl)benzofuran-4-yl, benzamid-2-yl, —NH₂, tert-butyl, and thien-2-yl.

Certain examples of compounds that may be useful in this invention are presented below in Table 1A and 1B. It is to be understood that the illustration of these compounds is in no way limiting the invention to the compounds described in these tables and it is therefore contemplated that other compounds are suitable for use in this invention. It should also be noted that the following compounds may exhibit stereoisomerism (i.e., E and Z isomers) and the invention contemplates use of either isomer and mixtures thereof.

TABLE 1A

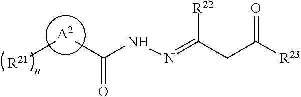

| No. | Structure | Compound Name |
|---|---|---|
| 1 | 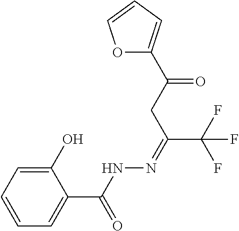 | 2-hydroxy-N′-(1,1,1-trifluoro-4-(furan-2-yl)-4- oxobutan-2-ylidene)benzohydrazide |
| 2 | 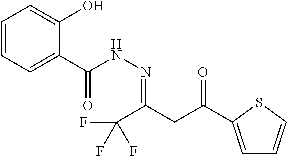 | 2-hydroxy-N′-(1,1,1-trifluoro-4-oxo-4-(thiophen- 2-yl)butan-2-ylidene)benzohydrazide |
| 3 | 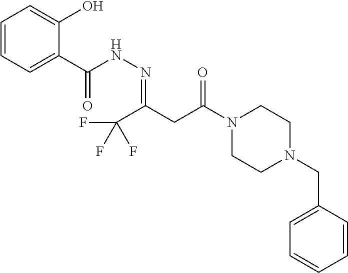 | N′-(4-(4-benzylpiperazin-1-yl)-1,1,1-trifluoro-4- oxobulan-2-ylidene)-2-hydroxy-benzohydrazide |
| 4 | 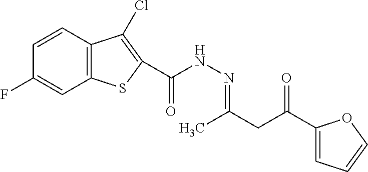 | 3-chloro-6-fluoro-N′-(4-(furan-2-yl)-4-oxobutan- 2-ylidene)benzo[b]-thiophene-2-carbohydrazide |
| 5 | 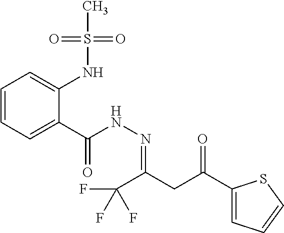 | N-(2-(2-(1,1,1-trifluoro-4-oxo-4- (thiophen-2-yl)butan-2-ylidene)hydrazine- carbonyl)phenyl)methanesulfonamide |
| 6 | 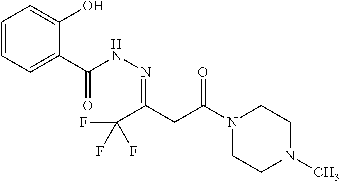 | 2-hydroxy-N′-(1,1,1-trifluoro-4-(4- methylpiperazin-1-yl)-4-oxobutan-2-ylidene) benzohydrazide |
| 7 | 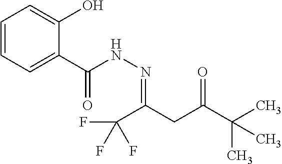 | 2-hydroxy-N′-(1,1,1-trifluoro-5,5-dimethyl-4- oxohexan-2-ylidene)benzohydrazide |
| 8 | 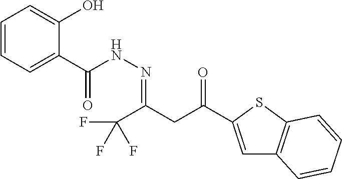 | N′-(4-(benzo[b]thiophen-2-yl)-1,1,1-trifluoro-4- oxobutan-2-ylidene)-2-hydroxy benzohydrazide |
| 9 | 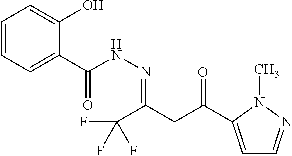 | 2-hydroxy-N′-(1,1,1-trifluoro-4-(1-methyl-1H- pyrazol-5-yl)-4-oxobutan-2-ylidene) benzohydrazide |
| 10 | 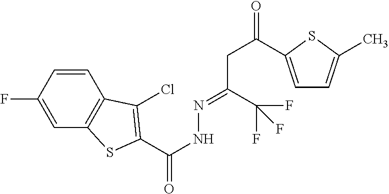 | 3-chloro-6-fluoro-N′-(1,1,1-trifluoro-4-(5- methylthiophen-2-yl)-4-oxobutan-2- ylidene)benzo[b]thiophene-2-carbohydrazide |
| 11 | 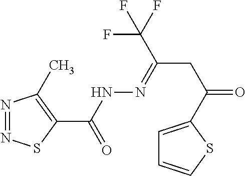 | 4-methyl-N′- (1,1,1-trifluoro-4-oxo-4-(thiophen-2- yl)butan-2-ylidene)-1,2,3-thiadiazole-5- carbohydrazide |
| 12 | 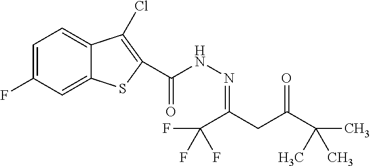 | 3-chloro-6-fluoro-N′-(1,1,1-trifluoro-5,5- dimethyl-4-oxohexan-2- ylidene)benzo[b]thiophene-2-carbohydrazide |
| 13 | 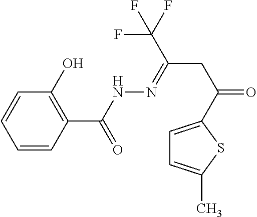 | 2-hydroxy-N′-(1,1,1-trifluoro-4-(5- methylthiophen-2-yl)-4-oxobutan-2-ylidene) benzohydrazide |
| 14 | 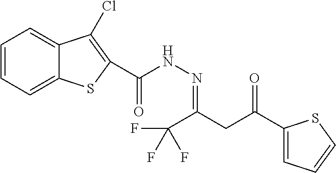 | 3-chloro-N′-(1,1,1-trifluoro-4-oxo-4-(thiophen-2- yl)butan-2-ylidene)benzo[b]thiophene-2- carbohydrazide |
| 15 | 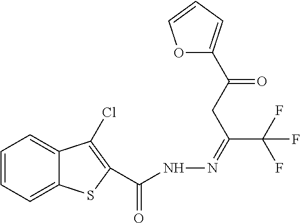 | 3-chloro-N′-(1,1,1-trifluoro-4-(furan-2-yl)-4- oxobutan-2-ylidene)benzo[b]thiophene-2- carbohydrazide |
| 16 | 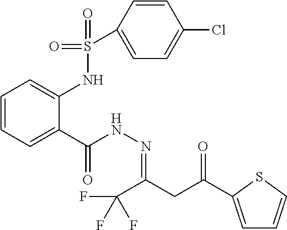 | 4-chloro-N-(2-(2-(1,1,1-trifluoro-4-oxo-4- (thiophen-2-yl)butan-2-ylidene) hydrazinecarbonyl)phenyl)benzenesulfonamide |
| 17 | 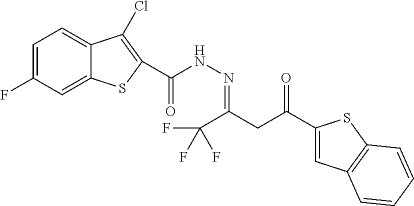 | N′-(4-(benzo[b]thiophen-2-yl)-1,1,1-trifluoro-4- oxobutan-2-ylidene)-3-chloro-6-fluorobenzo[b] thiophene-2-carbohydrazide |
| 18 | 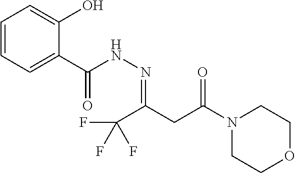 | 2-hydroxy-N′-(1,1,1-trifluoro-4-morpholino-4- oxobutan-2-ylidene)benzohydrazide |
| 19 | 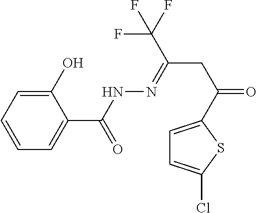 | N′-(4-(5-chlorothiophen-2-yl)-1,1,1-trifluoro-4- oxobutan-2-ylidene)-2-hydroxy benzohydrazide |
| 20 | 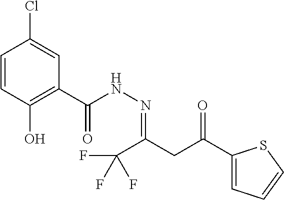 | 5-chloro-2-hydroxy-N′-(1,1,1-trifluoro-4-oxo-4- (thiophen-2-yl)butan-2-ylidene)benzohydrazide |
| 21 | 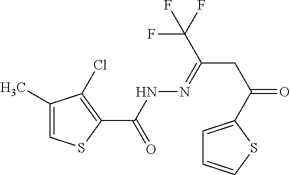 | 3-chloro-4-methyl-N′-(1,1,1-trifluoro-4-oxo-4- (thiophen-2-yl)butan-2-ylidene)thiophene-2- carbohydrazide |
| 22 | 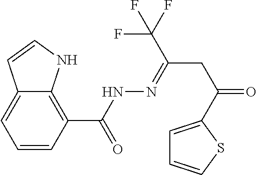 | N′-(1,1,1-trifluoro-4-oxo-4-(thiophen-2-yl)butan- 2-ylidene)-1H-indole-7-carbohydrazide |
| 23 | 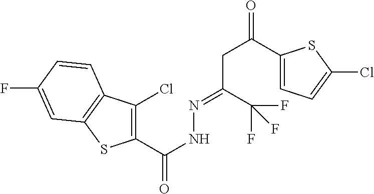 | 3-chloro-N′-(4-(5-chlorothiophen-2-yl)-1,1,1- trifluoro-4-oxobulan-2-ylidene)-6- fluorobenzo[b]thiophene-2-carbohydrazide |
| 24 | 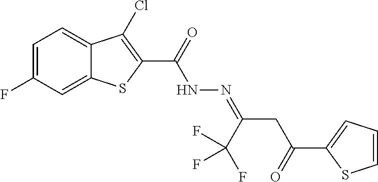 | 3-chloro-6-fluoro-N′-(1,1,1-trifluoro-4-oxo-4- (thiophen-2-yl)butan-2- ylidene)benzo[b]thiophene-2-carbohydrazide |
| 25 | 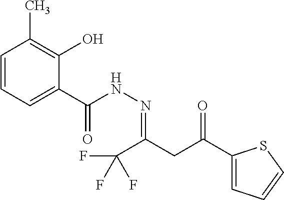 | 2-hydroxy-3-methyl-N′-(1,1,1-trifluoro-4-oxo-4- (thiophen-2-yl)butan-2-ylidene)benzohydrazide |
| 26 | 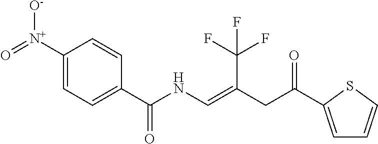 | 4-nitro-N′-(1,1,1-trifluoro-4-oxo-4-(thiophen-2- yl)butan-2-ylidene)benzohydrazide |
| 27 | 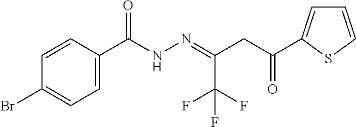 | 4-bromo-N′-(1,1,1-trifluoro-4-oxo-4-(thiophen-2- yl)butan-2-ylidene)benzohydrazide |
| 28 | 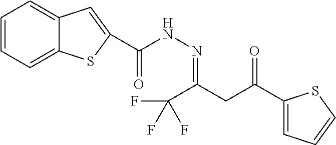 | N′-(1,1,1-trifluoro-4-oxo-4-(thiophen-2-yl)butan- 2-ylidene)benzo[b]thiophene-2-carbohydrazide |
| 29 | 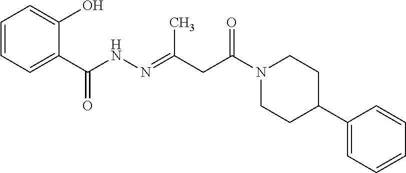 | 2-hydroxy-N′-(4-oxo-4-(4-phenylpiperidin-1- yl)butan-2-ylidene)benzohydrazide |
| 30 | 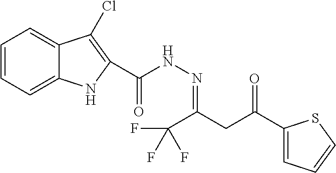 | 3-chloro-N′-(1,1,1-trifluoro-4-oxo-4-(thiophen-2- yl)butan-2-ylidene)-1H-indole-2-carbohydrazide |
| 31 | 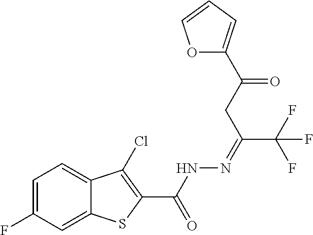 | 3-chloro-6-fluoro-N′-(1,1,1-trifluoro-4-(furan-2- yl)-4-oxobulan-2-ylidene)benzo[b]thiophene-2- carbohydrazide |
| 32 | 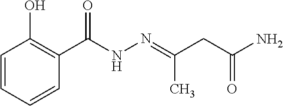 | 3-(2-(2-hydroxybenzoyl)hydrazono)butanamide |
| 33 | 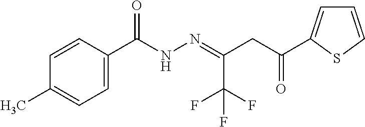 | 4-methyl-N′- (1,1,1-trifluoro-4-oxo-4-(thiophen-2- yl)butan-2-ylidene)benzohydrazide |
| 34 | 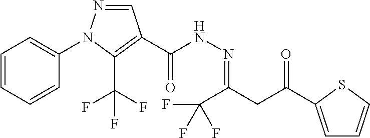 | 1-phenyl-N′-(1,1,1-trifluoro-4-oxo-4-(thiophen-2- yl)butan-2-ylidene)-5-(trifluoromethyl)-1H- pyrazole-4-carbohydrazide |
| 35 | 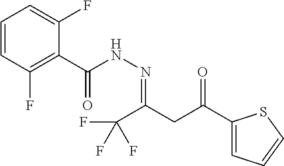 | 2,6-difluoro-N′-(1,1,1-trifluoro-4-oxo-4- (thiophen-2-yl)butan-2-ylidene)benzohydrazide |

TABLE 1B

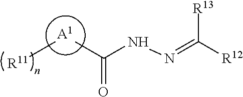

| No. | Structure | Compound Name |
|---|---|---|
| 36 | 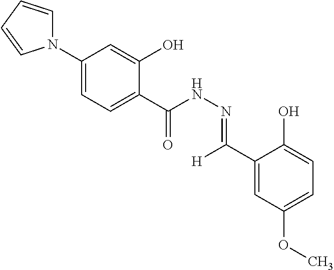 | 2-hydroxy-N′-(2-hydroxy-5-methoxy- benzylidene)-4-(1H-pyrrol-1-yl) benzohydrazide |
| 37 | 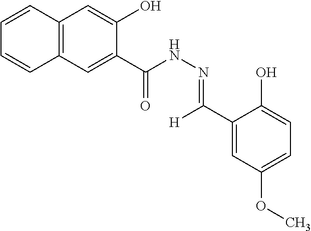 | 3-hydroxy-N′-(2-hydroxy-5-methoxy- benzylidene)-2-naphtho hydrazide |
| 38 | 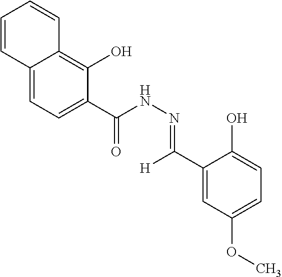 | 1-hydroxy-N′-(2-hydroxy-5- methoxybenzylidene)-2-naphtho- hydrazide |
| 39 | 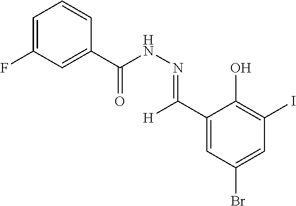 | N′-(5-bromo-2-hydroxy-3- iodobenzylidene)-3-fluoro- benzohydrazide |
| 40 | 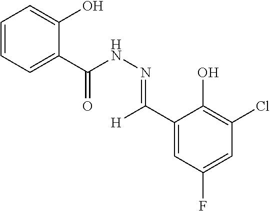 | N′-(3-chloro-5-fluoro-2- hydroxybenzylidene)-2-hydroxy- benzohydrazide |
| 41 | 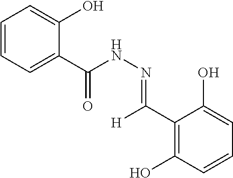 | N′-(2,6-dihydroxybenzylidene)-2- hydroxy benzohydrazide |
| 42 | 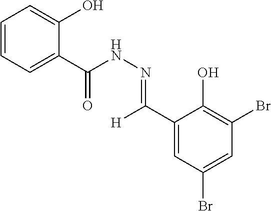 | N′-(3,5-dibromo-2-hydroxybenzylidene)- 2-hydroxybenzohydrazide |
| 43 | 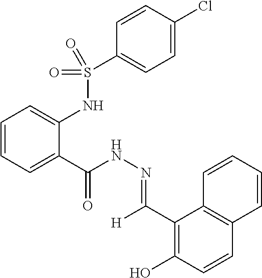 | 4-chloro-N-(2-(2-((2- hydroxynaphthalen-1- yl)methylene)hydrazinecarbonyl)phenyl) benzenesulfonamide |
| 44 | 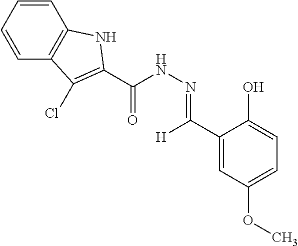 | 3-chloro-N′-(2-hydroxy-5- methoxybenzylidene)-1H-indole-2- carbohydrazide |
| 45 | 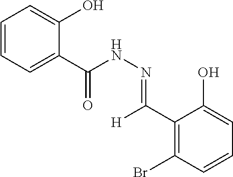 | N′-(2-bromo-6-hydroxybenzylidene)-2- hydroxybenzo-hydrazide |
| 46 | 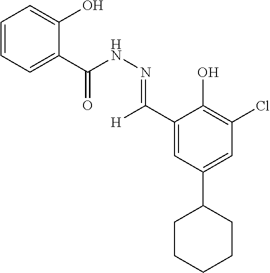 | N′-(3-chloro-5-cyclohexyl-2- hydroxybenzylidene)-2-hydroxybenzo- hydrazide |
| 47 | 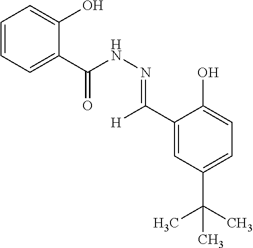 | N′-(5-tert-butyl-2-hydroxybenzylidene)- 2-hydroxy-benzohydrazide |
| 48 | 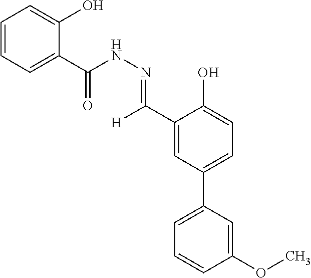 | 2-hydroxy-N′-((4-hydroxy-3′- methoxybiphenyl-3-yl)methylene) benzohydrazide |
| 49 | 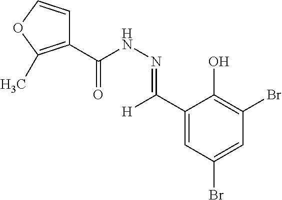 | N′-(3,5-dibromo-2-hydroxy- benzylidene)-2-methylfuran-3- carbohydrazide |
| 50 | 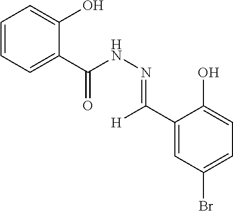 | N′-(5-bromo-2-hydroxy-benzylidene)-2- hydroxybenzohydrazide |
| 51 | 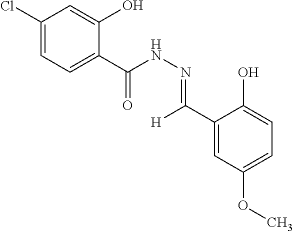 | 4-chloro-2-hydroxy-N′-(2-hydroxy-5- methoxy-benzylidene)benzohydrazide |
| 52 | 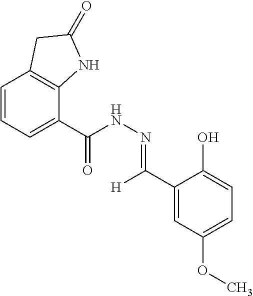 | N′-(2-hydroxy-5-methoxy-benzylidene)- 2-oxoindoline-7-carbohydrazide |
| 53 | 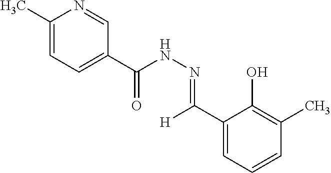 | N′-(2-hydroxy-3-methyl-benzylidene)-6- methylnicotino-hydrazide |
| 54 | 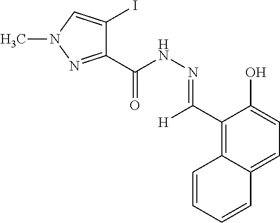 | N′-((2-hydroxy-naphthalen-1- yl)methylene)-4-iodo-1-methyl-1H- pyrazole-3-carbohydrazide |
| 55 | 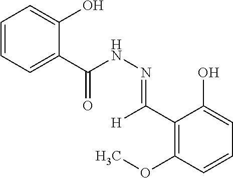 | 2-hydroxy-N′-(2-hydroxy-6-methoxy- benzylidene)benzohydrazide |
| 56 | 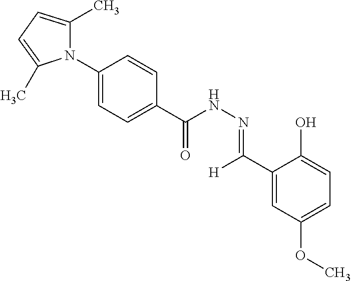 | 4-(2,5-dimethyl-1H-pyrrol-1-yl)-N′-(2- hydroxy-5-methoxy-benzylidene) benzohydrazide |
| 57 | 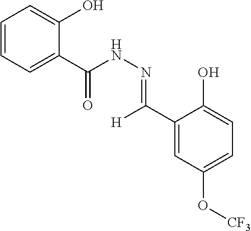 | 2-hydroxy-N′-(2-hydroxy-5-(trifluoro- methoxy)benzylidene)benzohydrazide |
| 58 | 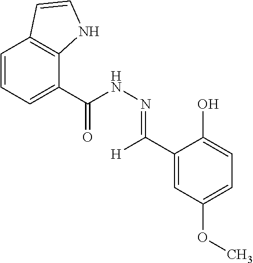 | N′-(2-hydroxy-5-methoxy-benzylidene)- 1H-indole-7-carbohydrazide |
| 59 | 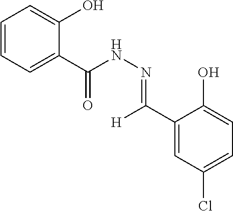 | N′-(5-chloro-2-hydroxy-benzylidene)-2- hydroxy-benzohydrazide |
| 60 | 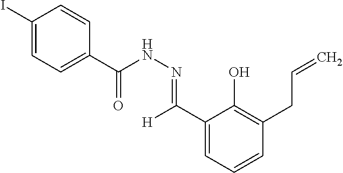 | N′-(3-allyl-2-hydroxy-benzylidene)-4- iodo-benzohydrazide |
| 61 | 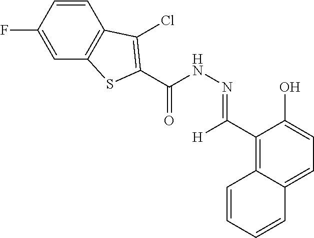 | 3-chloro-6-fluoro-N′-((2- hydroxynaphthalen-1-yl)methylene) benzo[b]thiophene-2-carbohydrazide |
| 62 | 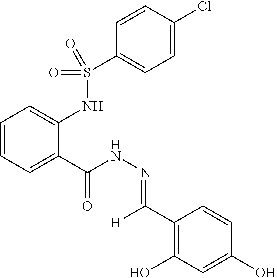 | 4-chloro-N-(2-(2-(2,4-dihydroxy- benzylidene)hydrazine- carbonyl)phenyl)benzene-sulfonamide |
| 63 | 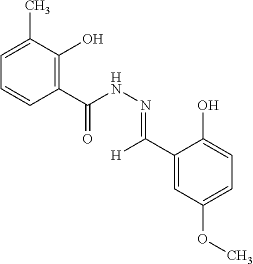 | 2-hydroxy-N′-(2-hydroxy-5-methoxy- benzylidene)-3-methyl-benzohydrazide |
| 64 | 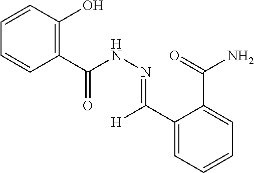 | 2-((2-(2-hydroxybenzoyl)hydrazono) methyl)benzamide |
| 65 | 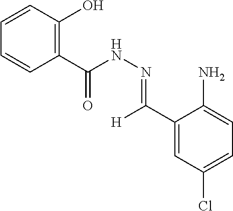 | N′-(2-amino-5-chloro-benzylidene)-2- hydroxy-benzohydrazide |
| 66 | 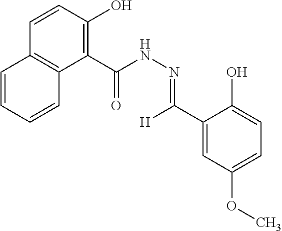 | 2-hydroxy-N′-(2-hydroxy-5-methoxy- benzylidene)-1-naphtho-hydrazide |
| 67 | 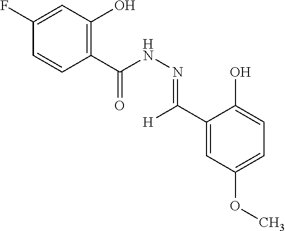 | 4-fluoro-2-hydroxy-N′-(2-hydroxy-5- methoxy-benzylidene)benzohydrazide |
| 68 | 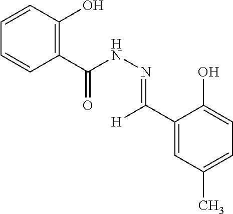 | 2-hydroxy-N′-(2-hydroxy-5-methyl- benzylidene)benzohydrazide |
| 69 | 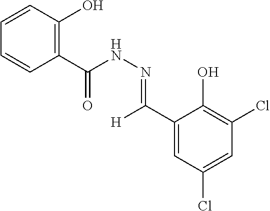 | N′-(3,5-dichloro-2-hydroxy- benzylidene)-2-hydroxy-benzohydrazide |
| 70 | 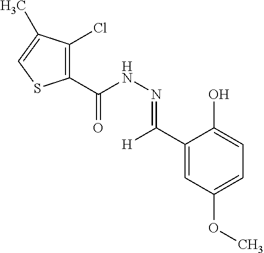 | 3-chloro-N′-(2-hydroxy-5-methoxy- benzylidene)-4-methylthiophene-2- carbohydrazide |
| 71 | 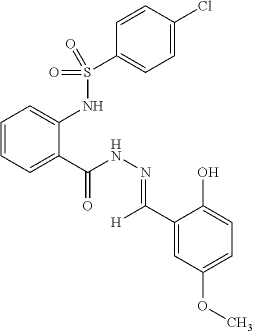 | 4-chloro-N-(2-(2-(2-hydroxy-5-methoxy- benzylidene)hydrazine- carbonyl)phenyl)benzene-sulfonamide |
| 72 | 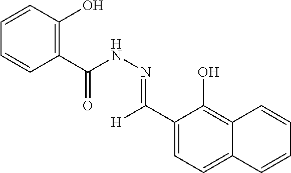 | 2-hydroxy-N′-((1-hydroxy-naphthalen-2- yl)methylene)benzohydrazide |
| 73 | 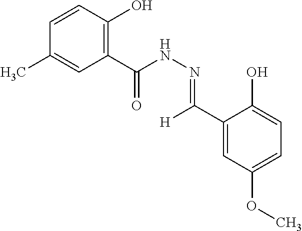 | 2-hydroxy-N′-(2-hydroxy-5-methoxy- benzylidene)-5-methyl-benzohydrazide |
| 74 | 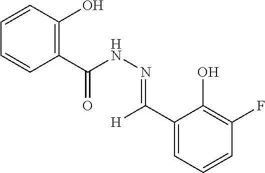 | N′-(3-fluoro-2-hydroxy-benzylidene)-2- hydroxy-benzohydrazide |
| 75 | 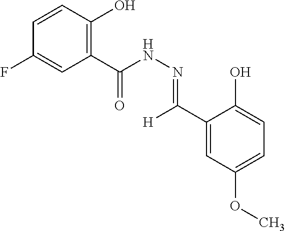 | 5-fluoro-2-hydroxy-N′-(2-hydroxy-5- methoxy-benzylidene)benzohydrazide |
| 76 | 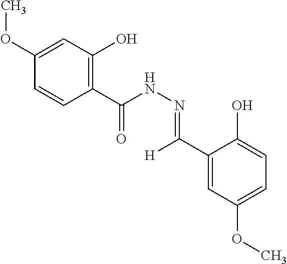 | 2-hydroxy-N′-(2-hydroxy-5-methoxy- benzylidene)-4-methoxy-benzohydrazide |
| 77 | 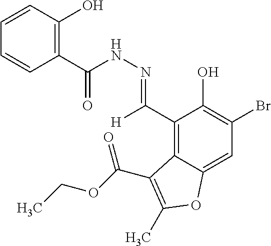 | ethyl 6-bromo-5-hydroxy-4-((2-(2- hydroxybenzoyl)hydrazono)methyl)-2- methyl-benzofuran-3-carboxylate |
| 78 | 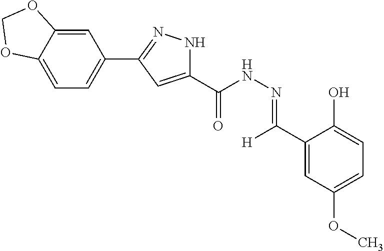 | 3-(benzo[d][1,3]dioxol-5-yl)-N′-(2- hydroxy-5-methoxy benzylidene)-1H- pyrazole-5-carbohydrazide |
| 79 | 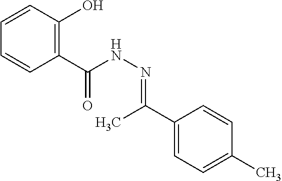 | 2-hydroxy-N′-(1-p-tolylethylidene) benzohydrazide |
| 80 | 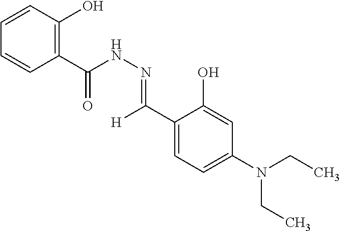 | N'-(4-(diethylamino)-2-hydroxy- benzylidene)-2-hydroxy-benzohydrazide |
| 81 | 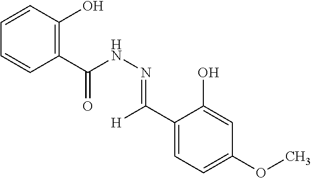 | 2-hydroxy-N′-(2-hydroxy-4-methoxy- benzylidene)benzohydrazide |
| 82 | 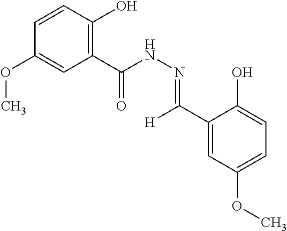 | 2-hydroxy-N′-(2-hydroxy-5-methoxy- benzylidene)-5-methoxy-benzohydrazide |
| 83 | 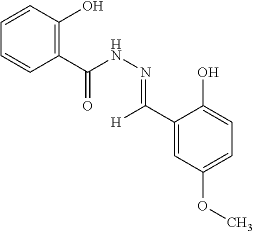 | 2-hydroxy-N′-(2-hydroxy-5-methoxy- benzylidene)benzohydrazide |
| 84 | 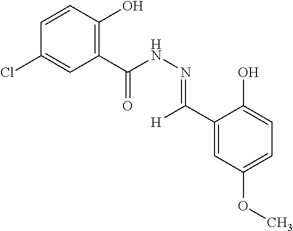 | 5-chloro-2-hydroxy-N′-(2-hydroxy-5- methoxy-benzylidene)benzohydrazide |
| 85 | 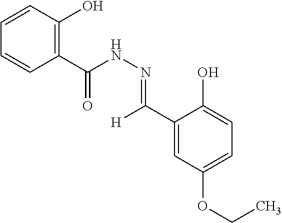 | N′-(5-ethoxy-2-hydroxy-benzylidene)-2- hydroxy-benzohydrazide |
| 86 | 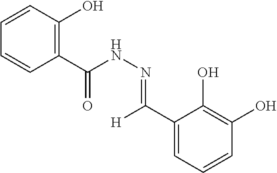 | N′-(2,3-dihydroxy-benzylidene)-2- hydroxy-benzohydrazide |
| 87 | 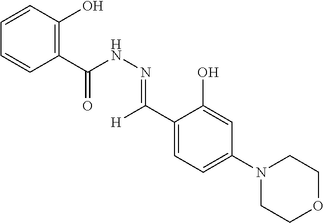 | 2-hydroxy-N′-(2-hydroxy-4-morpholino- benzylidene)-benzohydrazide |
| 88 | 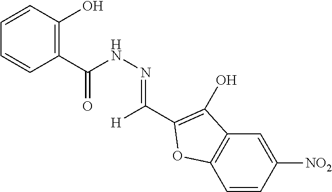 | 2-hydroxy-N′-((3-hydroxy-5-nitro- benzofuran-2-yl)methylene) benzohydrazide |
| 89 | 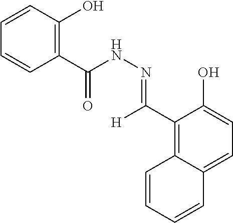 | 2-hydroxy-N′-((2-hydroxy-naphthalen-1- yl)methylene)benzohydrazide |
| 90 | 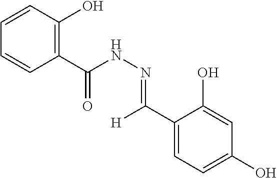 | N′-(2,4-dihydroxybenzylidene)-2- hydroxy-benzohydrazide |
| 91 | 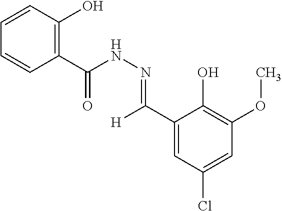 | N′-(5-chloro-2-hydroxy-3-methoxy- benzylidene)-2-hydroxy-benzohydrazide |
| 92 | 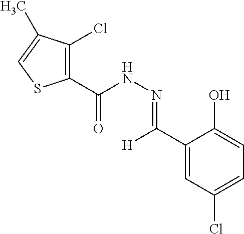 | 3-chloro-N′-(5-chloro-2- hydroxybenzylidene)-4-methylthiophene- 2-carbohydrazide |
| 93 | 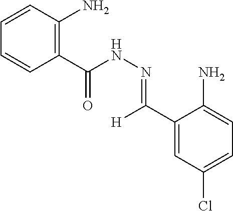 | 2-amino-N′-(2-amino-5- chlorobenzylidene)benzohydrazide |
| 94 | 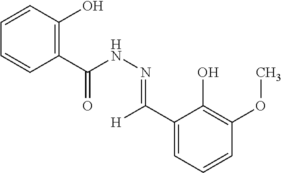 | 2-hydroxy-N′-(2-hydroxy-3- methoxybenzylidene)benzohydrazide |
| 95 | 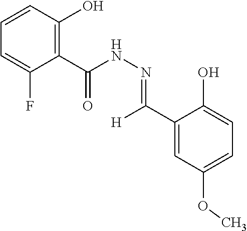 | 2-fluoro-6-hydroxy-N′-(2-hydroxy-5- methoxybenzylidene)benzohydrazide |
| 96 | 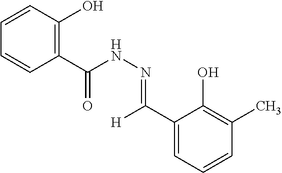 | 2-hydroxy-N′-(2-hydroxy-3- methylbenzylidene)benzohydrazide |
| 97 | 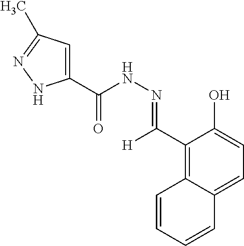 | N′-((2-hydroxynaphthalen-1- yl)methylene)-3-methyl-1H-pyrazole-5- carbohydrazide |
| 98 | 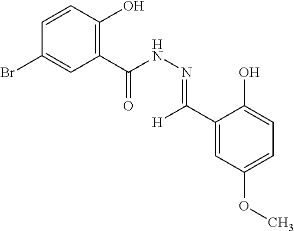 | 5-bromo-2-hydroxy-N′-(2-hydroxy-5- methoxybenzylidene)benzohydrazide |
| 99 | 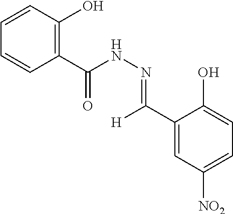 | 2-hydroxy-N′-(2-hydroxy-5- nitrobenzylidene)benzohydrazide |
| 100 | 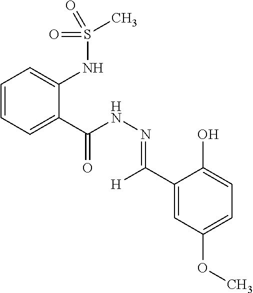 | N-(2-(2-(2-hydroxy-5- methoxybenzylidene)hydrazinecarbonyl) phenyl)methanesulfonamide |
| 101 | 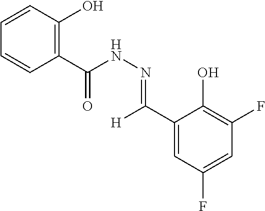 | N′-(3,5-difluoro-2-hydroxybenzylidene)- 2-hydroxybenzohydrazide |
| 102 | 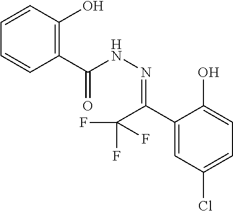 | N′-(1-(5-chloro-2-hydroxyphenyl)-2,2,2- trifluoroethylidene)-2- hydroxybenzohydrazide |
| 103 | 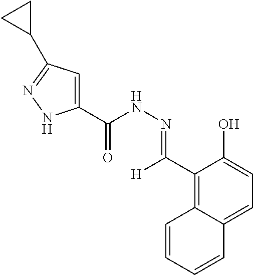 | 3-cyclopropyl-N′-((2- hydroxynaphthalen-1-yl)methylene)-1H- pyrazole-5-carbohydrazide |
| 104 | 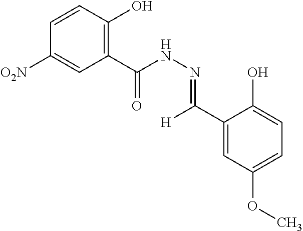 | 2-hydroxy-N′-(2-hydroxy-5- methoxybenzylidene)-5- nitrobenzohydrazide |
| 105 | 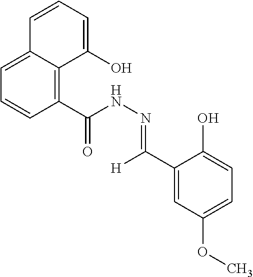 | 8-hydroxy-N′-(2-hydroxy-5- methoxybenzylidene)-1- naphthohydrazide |
| 106 | 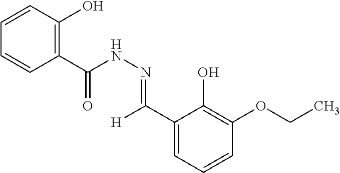 | N′-(3-ethoxy-2-hydroxybenzylidene)-2- hydroxybenzohydrazide |
| 107 | 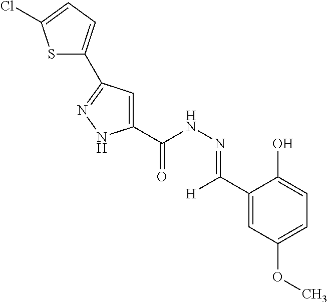 | 3-(5-chlorothiophen-2-yl)-N′-(2- hydroxy-5-methoxybenzylidene)-1H- pyrazole-5-carbohydrazide |
| 108 | 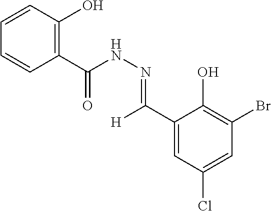 | N′-(3-bromo-5-chloro-2- hydroxybenzylidene)-2- hydroxybenzohydrazide |
| 109 | 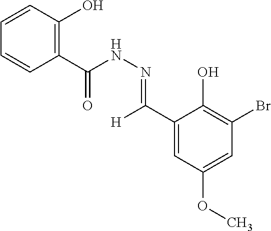 | N′-(3-bromo-2-hydroxy-5- methoxybenzylidene)-2- hydroxybenzohydrazide |
| 110 | 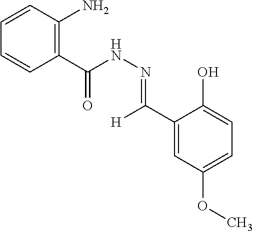 | 2-amino-N′-(2-hydroxy-5- methoxybenzylidene)benzohydrazide |
| 111 | 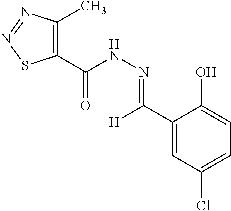 | N′-(5-chloro-2-hydroxybenzylidene)-4- methyl-1,2,3-thiadiazole-5- carbohydrazide |
| 112 | 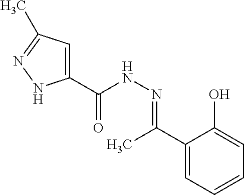 | N′-(1-(2-hydroxyphenyl)ethylidene)-3- methyl-1H-pyrazole-5-carbohydrazide |
| 113 | 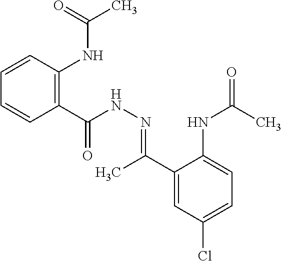 | N-(2-(2-(2-acetamido-5- chlorobenzylidene)hydrazinecarbonyl) phenyl)acetamide |
| 114 | 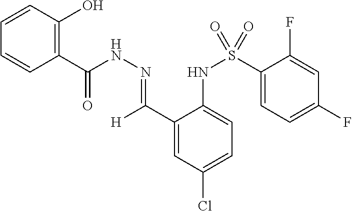 | N-(4-chloro-2-((2-(2- hydroxybenzoyl)hydrazono)methyl) phenyl)-2,4-difluorobenzenesulfonamide |
| 115 | 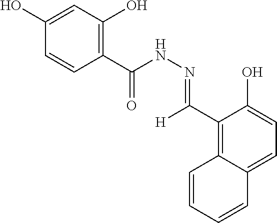 | 2,4-dihydroxy-N′-((2- hydroxynaphthalen-1- yl)methylene)benzohydrazide |
| 116 | 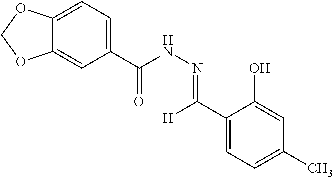 | N′-(2-hydroxy-4- methylbenzylidene)benzo[d][1,3]dioxole- 5-carbohydrazide |

### 2. Chemical Definitions

“Alkyl” refers to monovalent saturated aliphatic hydrocarbyl groups having from 1 to 10 carbon atoms and, in some embodiments, from 1 to 6 carbon atoms. “C_{x-y}alkyl” refers to alkyl groups having from x to y carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 and, in some embodiments, 1 to 3 or 1 to 2 substituents selected from the group consisting of alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Alkylidene” or “alkylene” refers to divalent saturated aliphatic hydrocarbyl groups having from 1 to 10 carbon atoms and, in some embodiments, from 1 to 6 carbon atoms. “(Cᵤ₋ᵥ)alkylene” refers to alkylene groups having from u to v carbon atoms. The alkylidene or alkylene groups include branched and straight chain hydrocarbyl groups. For example “(C₁₋₆)alkylene” is meant to include methylene, ethylene, propylene, 2-methypropylene, pentylene, and the like.

The term “aralkyl” refers to the term aryl-alkylene wherein alkylene is as defined above and aryl is as defined below. Examples of this group include, but are not limited to benzyl, phenethyl, and the like.

“Substituted alkylidene” or “substituted alkylene” refers to an alkylidene group having from 1 to 5 and, in some embodiments, 1 to 3 or 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, SO₃H, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, 1,3-butadienyl, and the like.

“Substituted alkenyl” refers to alkenyl groups having from 1 to 3 substituents and, in some embodiments, 1 to 2 substituents selected from the group consisting of alkoxy, substituted alkoxy, acyl, acylamino, acyloxy, alkyl, substituted alkyl, alkynyl, substituted alkynyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Substituted alkynyl” refers to alkynyl groups having from 1 to 3 substituents and, in some embodiments, from 1 to 2 substituents selected from the group consisting of alkoxy, substituted alkoxy, acyl, acylamino, acyloxy, alkyl, substituted alkyl, alkenyl, substituted alkenyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, substituted hydrazino, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR²¹R²² where R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonyl” refers to the group —C(S)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonylamino” refers to the group —NR²⁰ (O)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonyloxy” refers to the group —O—C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyl” refers to the group —SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyloxy” refers to the group —O—SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amidino” refers to the group —C(═NR²⁵)NR²³R²⁴ where R²⁵, R²³, and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

The term “biaryl” refers to the group aryl-arylene where the term aryl is as noted above and the term arylene refers to a divalent aryl group. Examples of this include, but are not limited to, biphenyl.

“Substituted aryl” refers to aryl groups which are substituted with 1 to 8 and, in some embodiments, 1 to 5, 1 to 3, or 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthyloxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from the group consisting of hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)β-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)β-cycloalkyl, —NR²⁰—C(O)O-substituted cycloalkyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)β-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms.

“Cycloalkenyl” refers to a partially saturated cycloalkyl ring having at least one site of >C═C< ring unsaturation.

“Substituted cycloalkyl” refers to a cycloalkyl group, as defined herein, having from 1 to 8, or 1 to 5, or in some embodiments 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are as defined herein. The term “substituted cycloalkyl” includes substituted cycloalkenyl groups.

“Cycloalkyloxy” refers to —O-cycloalkyl wherein cycloalkyl is as defined herein.

“Substituted cycloalkyloxy” refers to —O-(substituted cycloalkyl) wherein substituted cycloalkyl is as defined herein.

“Cycloalkylthio” refers to —S-cycloalkyl wherein cycloalkyl is as defined herein.

“Substituted cycloalkylthio” refers to —S-(substituted cycloalkyl).

“Guanidino” refers to the group —NHC(═NH)NH₂.

“Substituted guanidino” refers to —NR²⁹C(═NR²⁹)N(R²⁹)₂ where each R²⁹ is independently selected from the group consisting of hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo, and iodo.

“Haloalkyl” refers to substitution of alkyl groups with 1 to 5 or in some embodiments 1 to 3 halo groups.

“Haloalkoxy” refers to substitution of alkoxy groups with 1 to 5 or in some embodiments 1 to 3 halo groups.

“Hydroxy” or “hydroxyl” refers to the group —OH.

“Heteroaryl” refers to an aromatic group of from 1 to 14 carbon atoms and 1 to 6 heteroatoms selected from the group consisting of oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In one embodiment, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, benzothienyl, or oxindolyl.

“Substituted heteroaryl” refers to heteroaryl groups that are substituted with from 1 to 8 or in some embodiments 1 to 5, or 1 to 3, or 1 to 2 substituents selected from the group consisting of the substituents defined for substituted aryl.

“Heteroaryloxy” refers to —O-heteroaryl wherein heteroaryl is as defined herein.

“Substituted heteroaryloxy” refers to the group —O-(substituted heteroaryl) wherein substituted heteroaryl is as defined herein.

“Heteroarylthio” refers to the group —S-heteroaryl wherein heteroaryl is as defined herein.

“Substituted heteroarylthio” refers to the group —S-(substituted heteroaryl) wherein substituted heteroaryl is as defined herein.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from the group consisting of nitrogen, sulfur, or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen and/or sulfur atom(s) of the heterocyclic group are optionally oxidized to provide for the N-oxide, sulfinyl, sulfonyl moieties. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocyclic” or “Substituted heterocycle” or “substituted heterocycloalkyl” or “substituted heterocyclyl” refers to heterocyclic groups, as defined herein, that are substituted with from 1 to 5 or in some embodiments 1 to 3 of the substituents as defined for substituted cycloalkyl.

“Heterocyclyloxy” refers to the group —O-heterocyclyl wherein heterocyclyl is as defined herein.

“Substituted heterocyclyloxy” refers to the group —O-(substituted heterocyclyl) wherein substituted heterocyclyl is as defined herein.

“Heterocyclylthio” refers to the group —S-heterocyclyl wherein heterocyclyl is as defined herein.

“Substituted heterocyclylthio” refers to the group —S-(substituted heterocyclyl) wherein substituted heterocyclyl is as defined herein.

Examples of heterocycle and heteroaryl groups include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocycloalkyl” refers to a 3 to 10 member cyclic substituent formed by replacement of two hydrogen atoms at a common carbon atom with an alkylene group having 2 to 9 carbon atoms, as exemplified by the following structure wherein the methylene group shown here attached to bonds marked with wavy lines is substituted with a spirocycloalkyl group:

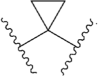

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any forms of the compounds within the generic and subgeneric formulae. Unless specified otherwise, the term further includes the racemates, stereoisomers, and tautomers of the compound or compounds.

“Racemates” refers to a mixture of enantiomers.

“Solvate” or “solvates” of a compound refer to those compounds, where compounds is as defined above, that are bound to a stoichiometric or non-stoichiometric amount of a solvent. Solvates of a compound includes solvates of all forms of the compound. Preferred solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N-moiety such as pyrazoles, imidazoles, benzimidazoles, triazoles, and tetrazoles.

“Prodrug” refers to any derivative of a compound of the embodiments that is capable of directly or indirectly providing a compound of the embodiments or an active metabolite or residue thereof when administered to a subject. Particularly favored derivatives and prodrugs are those that increase the bioavailability of the compounds of the embodiments when such compounds are administered to a subject (e.g., by allowing an orally administered compound to be more readily absorbed into the blood) or which enhance delivery of the parent compound to a biological compartment (e.g., the brain or lymphatic system) relative to the parent species. Prodrugs include ester forms of the compounds of the invention. Examples of ester prodrugs include formate, acetate, propionate, butyrate, acrylate, and ethylsuccinate derivatives. An general overview of prodrugs is provided in T. Higuchi and V. Stella, Pro-drugs as Novel Delivery Systems, Vol. 14 of the A.C.S. Symposium Series, and in Edward B. Roche, ed., Bioreversible

Carriers in Drug Design, American Pharmaceutical Association and Pergamon Press, 1987, both of which are incorporated herein by reference.

For example in this invention, the term “prodrug” refers to compounds of formula I, II, or III that include chemical groups which, in vivo, can be converted to the carboxylate group, hydroxyl group, or amine group on the R³ group, the hydroxyl group or amine group on the R¹ group of the A ring or the amine of a heteroaryl A ring. Examples of such chemical groups which can act as prodrugs for carboxylates and hydroxyl groups are esters which can be chemically cleaved or enzymatically cleaved by esterases. Examples of such chemical groups which can act as prodrugs for amines are amides which can be enzymatically cleaved by proteases and phosphoryloxymethyl carbamates which can be enzymatically cleaved by alkaline phosphatases (see Safadi et al. Pharmaceutical Research 1993, 10 (9), 1350-1355).

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

### 3. Compound Selection

A preferred method of selecting compounds for their use in the methods of the invention involves assaying the compounds with Fluorescence Resonance Energy Transfer (FRET). FRET has been used to measure, detect, identify, assay, analyze, and characterize various interactions and processes in biological systems (see e.g., Mitra et al. (1996) Gene 173:13-17; De Angelis (1999) Physiol. Genomics. 21:93-99; Latif and Graves (2000) Thyroid 10(5):407-412; Rye (2001) Methods 24(3):278-288; Kenworthy (2001) Methods 24(3):289-296; Periasamy (2001) J. Biomed. Opt. 6(3):287-291; Truong and Ikura (2001) Curr. Opin. Struct. Biol. 11(5):573-578; Zhang et al. (2002) Nat. Rev. Mol. Cell. Biol. 3(12):906-918; Sitte and Freissmuth (2003) Eur. J. Pharmacol. 479:229-236; Milligan (2004) Eur. J. Pharm. Sci. 21(4):397-405; Herman et al. (2004) Methods Mol. Biol. 261:351-370; Roda et al. (2004) Trends Biotechnol. 22(6):295-303; Wallrabe and Periasamy (2005) Curr. Opin. Biotechnol. 16(1):19-27; Milligan and Bouvier (2005) FEBS J. 272(12):2914-2915; references in any of the foregoing; and the like).

FRET methods, protocols, techniques, assays, and the like are described generally and specifically in a number of patents and patent applications, including: U.S. Pat. No. 6,908,769; U.S. Pat. No. 6,824,990; U.S. Pat. No. 6,762,280; U.S. Pat. No. 6,689,574; U.S. Pat. No. 6,661,909; U.S. Pat. No. 6,642,001; U.S. Pat. No. 6,639,078; U.S. Pat. No. 6,472,156; U.S. Pat. No. 6,456,734; U.S. Pat. No. 6,376,257; U.S. Pat. No. 6,348,322; U.S. Pat. No. 6,323,039; U.S. Pat. No. 6,291,201; U.S. Pat. No. 6,280,981; U.S. Pat. No. 5,914,245; U.S. Pat. No. 5,661,035; references in any of the foregoing; and the like.

Similarly, fluorescence polarization (FP) has also been used to measure, detect, identify, assay, analyze, and characterize various interactions and processes in biological systems (see e.g., Lundblad et al. (1996) Mol. Endocrinol. 10(6):607-612; Nasir and Jolley (1999) Comb. Chem. High Throughput Screen. 2(4):177-190; Park and Raines (2004) Methods Mol. Biol. 261:161-166; references in any of the foregoing; and the like).

Fluorescence polarization (FP) methods, protocols, techniques, assays, and the like are described generally and specifically in a number of patents and patent applications, including: U.S. Pat. No. 6,794,158; U.S. Pat. No. 6,632,613; U.S. Pat. Nos. 6,630,295; 6,596,546; U.S. Pat. Nos. 6,569,628; 6,555,326; U.S. Pat. No. 6,511,815; U.S. Pat. No. 6,448,018; U.S. Pat. No. 6,432,632; U.S. Pat. No. 6,331,392; U.S. Pat. No. 6,326,142; U.S. Pat. No. 6,284,544; U.S. Pat. No. 6,207,397; U.S. Pat. No. 6,171,807; U.S. Pat. No. 6,066,505; U.S. Pat. No. 5,976,820; U.S. Pat. No. 5,804,395; U.S. Pat. No. 5,756,292; U.S. Pat. No. 5,445,935; U.S. Pat. No. 5,427,960; U.S. Pat. No. 5,407,834; U.S. Pat. No. 5,391,740; U.S. Pat. No. 5,315,015; U.S. Pat. No. 5,206,179; U.S. Pat. No. 5,070,025; U.S. Pat. No. 5,066,426; U.S. Pat. No. 4,952,691; U.S. Pat. No. 4,863,876; U.S. Pat. No. 4,751,190; U.S. Pat. No. 4,681,859; U.S. Pat. No. 4,668,640; U.S. Pat. No. 4,614,823; U.S. Pat. No. 4,585,862; U.S. Pat. No. 4,510,251; U.S. Pat. No. 4,476,229; U.S. Pat. No. 4,429,230; U.S. Pat. No. 4,420,568; U.S. Pat. No. 4,203,670; references in any of the foregoing; and the like.

FRET and FP have been used in the field of amyloid research (see e.g., U.S. Pat. No. 6,927,401; U.S. Pat. No. 6,906,104; U.S. Pat. No. 6,905,827; U.S. Pat. No. 6,881,546; U.S. Pat. No. 6,864,290; U.S. Pat. No. 6,864,103; U.S. Pat. No. 6,858,383; U.S. Pat. No. 6,846,813; U.S. Pat. No. 6,828,106; U.S. Pat. No. 6,803,188; U.S. Pat. No. 6,770,448; U.S. Pat. No. 6,713,276; U.S. Pat. No. 6,600,017; U.S. Pat. No. 6,515,113; U.S. Pat. No. 6,495,664; U.S. Pat. No. 6,323,039; U.S. Pat. No. 6,294,330; U.S. Pat. No. 6,280,981; U.S. Pat. No. 6,197,928; U.S. Pat. No. 5,981,200; Kim and Lee (2004) Biochem. Biophys. Res. Commun. 316(2):393-397; Bacskai et al. (2003) J. Biomed. Opt. 8(3):368-375; Gorman, P. M. et al. (2003) J. Mol. Biol. 325(4):743-757; Garzon-Rodrequez et al. (1997) J. Biol. Chem. 272(34):21037-21044; Lindgren et al. (2005) Biophys. J. 88(6):4200-4212; Lewis et al. (2004) Neurobiol. Aging 25(9):1175-1185; Leissring et al. (2003) J. Biol. Chem. 278(39):37314-37320; Taylor et al. (2003) J. Protein Chem. 22(1):31-40; Allsop et al. (2001) Biochem. Soc. Symp. 67:1-14; Allsop et al. (2001) Biochem. Biophys. Res. Commun. 285(1):58-63; Huang et al. (2000) J. Biol. Chem. 275(46):36436-36440; references in any of the foregoing; and the like).

### 4. Applications of FRET for Compound Discovery

Further compounds useful in the methods of the invention may be evaluated by the FRET assay described in Example 16. This assay was used on a variety of commercially available compound libraries to assess the specific inhibition formation of soluble amyloid β₁₋₄₂ oligomers of each compound. Lead compounds were identified and further compound libraries were obtained. In addition, structure activity relationships around lead compounds were conducted leading to still further enhancements in activity.

### D. Synthesis of Compounds of the Invention

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Second Edition, Wiley, New York, 1991, and references cited therein.

Furthermore, the compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Compounds of the invention were synthesized according to Examples provided below.

### D. Testing and Administration

Effective doses of the compositions of the present invention, for the treatment of the above described diseases, vary depending upon may different factors, including means of administration, physiological state of the patient, whether the patient is human or an animal, other medications administered, and whether treatment is prophylactic or therapeutic. Usually, the patient is a human, but in certain embodiments, a patient is an animal, particularly an animal selected from a mammalian species including rat, rabbit, bovine, ovine, porcine, canine, feline, murine, equine, and primate.

The compounds can be administered on multiple occasions, wherein intervals between single dosages can be daily, weekly, monthly, or yearly. Intervals can also be irregular as indicated by measuring blood levels of Aβ₁₋₄₂ protein or ADDLs, or ADDL complexes in the patient. Alternatively, one or more of the compounds of the invention can be administered as a sustained-release formulation, in which case less frequent administration is required. Dosage and frequency may vary depending on the half-life of the compounds of the invention. In therapeutic applications, a relatively high dosage at relatively short intervals is sometimes required until progression of the disease is reduced or terminated, and sometimes until the patient shows partial or complete amelioration of symptoms of the disease. Thereafter, the patient can be administered a prophylactic regime.

Administration of a pharmaceutical composition of on the compounds described herein can be carried out via a variety of routes including, but are not limited to, oral, topical, pulmonary, rectal, subcutaneous, intradermal, intranasal, intracranial, intramuscular, intraocular, or intra-articular injection and the like. The most typical route of administration is oral, although other routes can be equally effective.

One or more compounds described herein can optionally be administered in combination with other biological or chemical agents that are at least partly effective in treatment of a Aβ₁₋₄₂ associated disease. An example of such an agent is, but are not limited to, Aβ₁₋₄₂ targeted antibodies as described in International Application Nos.: WO 2003/253673; WO 2006/014478, U.S. Pat. No. 2,489,195, US Publication No. 2007-0048312, and U.S. application Ser. No. 11/571,532, which are incorporated herein by reference.

The compounds described herein may be administered to a patient in an amount sufficient to inhibit, regulate and/or modulate the formation of neurotoxic ADDLs or the activity of such ligands in said patient. A skilled clinician would be able to readily ascertain appropriate amounts of the compounds described here to effectively inhibit, regulate and/or modulate the formation of neurotoxic ADDLs or the activity of such ligands in said patient. Contemplated amounts of the compounds described herein include for example, but are not limited to, from about 0.05 to 2000 mg/m²/day of one compound or more than one compound.

As noted above, the compounds described herein may be administered for example, but are not limited to, orally, topically, pulmonaryl), rectally, subcutaneously, intradermally, intranasally, intracranially, intramuscularly, intraocularly, or intra-arterially and the like. The carrier or excipient or excipient mixture can be a solvent or a dispersive medium containing for example, but are not limited to, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Moreover, other or supplementary active ingredients can also be incorporated into the final composition.

Diseases which are treated by the methods described herein include Alzheimer's disease, Down's syndrome, stroke, mild cognitive impairment, focal ischemia associated dementia and neuronal degeneration.

### E. Pharmaceutical Formulations and Routes of Administration

When employed as pharmaceuticals, the compounds of this invention are usually administered in the form of pharmaceutical compositions. These compounds can be administered by a variety of routes including oral, topical, pulmonary, rectal, subcutaneous, intradermal, intranasal, intracranial, intramuscular, intraocular, or intra-articular injection. These compounds are effective as both injectable and oral compositions. Such compositions are prepared in a manner well known in the pharmaceutical art and comprise at least one active compound.

This invention also includes pharmaceutical compositions which contain, as the active ingredient, one or more of the compounds described herein associated with pharmaceutically acceptable carriers. In making the compositions of this invention, the active ingredient is usually mixed with an excipient, diluted by an excipient or enclosed within such a carrier which can be in the form of a capsule, sachet, paper or other container. The excipient employed is typically an excipient suitable for administration to patient. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

Administration of therapeutic agents by intravenous formulation is well known in the pharmaceutical industry. An intravenous formulation should possess certain qualities aside from being just a composition in which the therapeutic agent is soluble. For example, the formulation should promote the overall stability of the active ingredient(s), also, the manufacture of the formulation should be cost effective. All of these factors ultimately determine the overall success and usefulness of an intravenous formulation.

Other accessory additives that may be included in pharmaceutical formulations of compounds of the present invention as follow: solvents: ethanol, glycerol, propylene glycol; stabilizers: ethylene diamine tetraacetic acid (EDTA), citric acid; antimicrobial preservatives: benzyl alcohol, methyl paraben, propyl paraben; buffering agents: citric acid/sodium citrate, potassium hydrogen tartrate, sodium hydrogen tartrate, acetic acid/sodium acetate, maleic acid/sodium maleate, sodium hydrogen phthalate, phosphoric acid/potassium dihydrogen phosphate, phosphoric acid/disodium hydrogen phosphate; and tonicity modifiers: sodium chloride, mannitol, dextrose.

The presence of a buffer may be necessary to maintain the aqueous pH in the range of from about 4 to about 8 and more preferably in a range of from about 4 to about 6. The buffer system is generally a mixture of a weak acid and a soluble salt thereof, e.g., sodium citrate/citric acid; or the monocation or dication salt of a dibasic acid, e.g., potassium hydrogen tartrate; sodium hydrogen tartrate, phosphoric acid/potassium dihydrogen phosphate, and phosphoric acid/disodium hydrogen phosphate.

The amount of buffer system used is dependent on (1) the desired pH; and (2) the amount of drug. Generally, the amount of buffer used is in a 0.5:1 to 50:1 mole ratio of buffer:drug (where the moles of buffer are taken as the combined moles of the buffer ingredients, e.g., sodium citrate and citric acid) of formulation to maintain a pH in the range of 4 to 8 and generally, a 1:1 to 10:1 mole ratio of buffer (combined) to drug present is used.

One useful buffer in the invention is sodium citrate/citric acid in the range of 5 to 50 mg per mL of sodium citrate to 1 to 15 mg per mL of citric acid, sufficient to maintain an aqueous pH of 4-6 of the composition.

The buffer agent may also be present to prevent the precipitation of the drug through soluble metal complex formation with dissolved metal ions, e.g., Ca, Mg, Fe, Al, Ba, which may leach out of glass containers or rubber stoppers or be present in ordinary tap water. The agent may act as a competitive complexing agent with the drug and produce a soluble metal complex leading to the presence of undesirable particulates.

In addition, the presence of an agent, e.g., sodium chloride in an amount of about of 1-8 mg/mL, to adjust the tonicity to the same value of human blood may be required to avoid the swelling or shrinkage of erythrocytes upon administration of the intravenous formulation leading to undesirable side effects such as nausea or diarrhea and possibly to associated blood disorders. In general, the tonicity of the formulation matches that of human blood which is in the range of 282 to 288 mOsm/kg, and in general is 285 mOsm/kg, which is equivalent to the osmotic pressure corresponding to a 0.9% solution of sodium chloride.

The intravenous formulation can be administered by direct intravenous injection, i.v. bolus, or can be administered by infusion by addition to an appropriate infusion solution such as 0.9% sodium chloride injection or other compatible infusion solution.

The compositions can be formulated in an oral unit dosage form. The term “unit dosage forms” refers to physically discrete units suitable as unitary dosages for a patient, each unit containing a predetermined quantity of active material calculated to produce the desired therapeutic effect, in association with a suitable pharmaceutical excipient.

The active compound is effective over a wide dosage range and is generally administered in a pharmaceutically effective amount. It, will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like. One of skill in the art, based on Example 17, would be able to readily assess the appropriate concentration of active compound to provide the necessary cognitive restoration.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.05 to about 2000 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices which deliver the formulation in an appropriate manner.

The following formulation examples illustrate the contemplated pharmaceutical compositions of the present invention.

### Formulation Example 1

Hard gelatin capsules containing the following ingredients are prepared:

| Ingredient | Quantity (mg/capsule) |
|---|---|
| Active Ingredient | 30.0 |
| Starch | 305.0 |
| Magnesium stearate | 5.0 |

The above ingredients are mixed and filled into hard gelatin capsules in 340 mg quantities.

### Formulation Example 2

A tablet formula is prepared using the ingredients below:

| Ingredient | Quantity (mg/tablet) |
|---|---|
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Formulation Example 3

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### Formulation Example 4

Tablets, each containing 30 mg of active ingredient, are prepared as follows:

| Ingredient | Quantity (mg/tablet) | |
|---|---|---|
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in sterile water) | | |
| Sodium carboxymethyl starch | 4.5 | mg |
| Magnesium stearate | 0.5 | mg |
| Talc | 1.0 | mg |

The active ingredient, starch, and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### Formulation Example 5

Capsules, each containing 40 mg of medicament are made as follows:

| Ingredient | Quantity (mg/capsule) | |
|---|---|---|
| Active Ingredient | 40.0 | mg |
| Starch | 109.0 | mg |
| Magnesium stearate | 1.0 | mg |
| Total | 150.0 | mg |

The active ingredient, starch and magnesium stearate are blended, passed through a No. 20 mesh U.S. sieve, and filled into hard gelatin capsules in 150 mg quantities.

### Formulation Example 6

Suppositories, each containing 25 mg of active ingredient are made as follows:

| Ingredient | Amount | |
|---|---|---|
| Active Ingredient | 25 | mg |
| Saturated fatty acid glycerides | 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 50 mg of medicament per 5.0 ml dose are made as follows:

| Ingredient | Amount | |
|---|---|---|
| Active Ingredient | 50.0 | mg |
| Xanthan gum | 4.0 | mg |
| Sodium carboxymethyl cellulose (11%) | | |
| Microcrystalline cellulose (89%) | 50.0 | mg |
| Sucrose | 1.75 | g |
| Sodium benzoate | 10.0 | mg |
| Flavor and Color | q.s. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

| Ingredient | Quantity (mg/capsule) | |
|---|---|---|
| Active Ingredient | 15.0 | mg |
| Starch | 407.0 | mg |
| Magnesium stearate | 3.0 | mg |
| Total | 425.0 | mg |

The active ingredient, starch, and magnesium stearate are blended, passed through a No. 20 mesh U.S. sieve, and filled into hard gelatin capsules in 425.0 mg quantities.

### Formulation Example 9

A subcutaneous formulation may be prepared as follows:

| Ingredient | Quantity |
|---|---|
| Active Ingredient | 5.0 mg |
| Corn Oil | 1.0 mL |

### Formulation Example 10

An intravenous formulation may be prepared as follows:

| Ingredient | Quantity | |
|---|---|---|
| Active Ingredient | 250 | mg |
| Isotonic saline | 1000 | mL |

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472, which is herein incorporated by reference.

Indirect techniques, usually involve formulating the compositions to provide for drug latentiation by the conversion of hydrophilic drugs into lipid-soluble drugs. Latentiation is generally achieved through blocking of the hydroxyl, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions, which can transiently open the blood-brain barrier.

Other suitable formulations for use in the present invention can be found in Remington's Pharmaceutical Sciences, Mace Publishing Company, Philadelphia, Pa., 17th ed. (1985).

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 each of which is incorporated herein by reference.

In prophylactic applications, compositions are administered to a patient at risk of developing AD (determined for example by genetic screening or familial trait) in an amount sufficient to inhibit the onset of symptoms of the disease. An amount adequate to accomplish this is defined as “prophylactically effective dose.” Amounts effective for this use will depend on the judgment of the attending clinician depending upon factors such as the age, weight and general condition of the patient, and the like.

As noted above, the compounds administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 and 8. It will be understood that use of certain of the foregoing excipients, carriers, or stabilizers will result in the formation of pharmaceutical salts.

The following examples are offered to illustrate this invention and are not to be construed in any way as limiting the scope of this invention. Unless otherwise stated, all temperatures are in degrees Celsius.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

In these examples and elsewhere, abbreviations have the following meanings:
μL or μl=microliter
μm=micrometer
AcOH=acetic acid
DCM=dichloromethane
DME ethylene glycol dimethyl ether
DMSO=dimethylsulfoxide
EtOH=ethanol
eq.=equivalents
g=gram
h=hour
HCl=hydrochloric acid
Hz=Hertz
LC=liquid chromatography
LiHMDS lithium hexamethyldisilazide
M=molar
MeOH=methanol
mg=milligram
MgSO₄=magnesium sulfate
min=minute
mL=milliliter
mM=millimolar
mm=millimeter
mmol=millimolar
mol=moles
MS=mass Spectroscopy
N=normal
nm=nanometer
NMR=nuclear magnetic resonance
μM=micromolar
SiO₂=silicon dioxide
TLC=thin layer chromatography
TMS=trimethylsilyl
THF=tetrahydrofuran
RT=room temperature
OMe=methoxy
Me=methyl
Pd(dppf)Cl₂=1,1′-Bis(diphenylphosphino)ferrocene]dichloropalladium(II)

Compounds of the invention are characterized by assessing melting points, mass spectroscopy, and/or nuclear magnetic resonance. Melting points are measured on a Fisher-Johns Melting point apparatus (Fisher cat 12-144) and are uncorrected. Mass spectrometric analysis is performed on an Agilent LC/MSD Multimode Instrument using the following parameters:
Source: ESI or APCI positive or negative mode, as noted for individual samples.
Sample Prep Typically 1 mg/mL in 1:4 DMSO/MeOH
Chromatography: Injection size 5 μL
Column: Diazem-Pfarma C18-A 5 μm, 4.6×50 mm
Solvents: A=H₂O with 0.1% Formic Acid modifier; B=Acetonitrile with 0.1% Formic Acid modifier
Flow rate: 1.0 mL/min
Gradient:

| Time (min) | Solvent Composition |
|---|---|
| 0 to 0.3 | 90% A/10% B |
| 0.3 to 2.5 | 90% A/10% B to 10% A/90% B |
| 2.5 to 4.5 | 10% A/90% B |
| 4.5 to 4.7 | 10% A/90% B to 90% A/10% B |
| 5.7 to 6.0 | 90% A/10% B |

Nuclear Magnetic resonance (NMR) analysis is performed with a Varian 400 MHz machine. The spectral reference is either TMS or the known chemical shift of the solvent. Some compounds are run at elevated temperature (i.e., 75° C.) to promote increased sample solubility.

### Example 1

### Preparation of Hydrazide Starting Materials

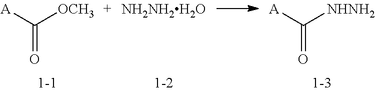

General Procedure: An ester 1-1 (10 mmol) was treated with hydrazine hydrate 1-2 (50 mmol) in refluxing 2-methoxyethanol until the reaction was completed as determined by TLC. The reaction mixture was cooled and water was added. The solution was concentrated until a precipitate formed. The precipitate was collected by vacuum filtration and washed with water, hexane, and ether-ethanol 9:1 and air dried to afford the Compound 1-3.

### 2-Hydroxy-5-nitrobenzohydrazide (1-4)

In analogy to the general procedure of Example 1, methyl 2-hydroxy-5-nitrobenzoate (10 mmol) was treated with hydrazine hydrate (50 mmol) in refluxing 2-methoxyethanol for 30 minutes. The reaction mixture was cooled whereupon a precipitate formed. The precipitate was collected by vacuum filtration and washed with ether-ethanol 9:1, water, and ether and air dried to afford Compound 1-4. LC-MS (APCI neg): 196 (M−H⁺).

### 2-Hydroxy-5-methoxybenzohydrazide (1-5)

Prepared from methyl 2-hydroxy-5-methoxy-benzoate in analogy to the general procedure of Example 1. LC-MS (APCI neg): 196 (M−H⁺).

### 2-Fluoro-6-hydroxybenzohydrazide (1-6)

Prepared from methyl 2-fluoro-6-hydroxy-benzoate in analogy to the general procedure of Example 1. Structure was confirmed by ¹H NMR (DMSO-d₆).

### 2-Hydroxy-4-(1H-pyrrol-1-yl)benzohydrazide (1-7)

Prepared from methyl 2-hydroxy-4-(1H-pyrrol-1-yl)benzoate in analogy to the general procedure of Example 1. LC-MS (APCI pos): 218.1 (M+H⁺).

### 3-Fluoro-6-hydroxybenzohydrazide (1-8)

Prepared from methyl 5-fluoro-2-hydroxybenzoate in analogy to the general procedure of Example 1. LCMS (APCI neg): 169 (M−H⁺).

### 4-Fluoro-6-hydroxybenzohydrazide (1-9)

Prepared from methyl 4-fluoro-2-hydroxybenzoate in analogy to the general procedure of Example 1. LCMS (APCI neg): 169 (M−H⁺).

### 2-Hydroxy-5-methylbenzohydrazide (1-10)

Prepared from methyl 5-fluoro-2-hydroxybenzoate in analogy to the general procedure of Example 1. LCMS (APCI pos): 167 (M+H⁺).

### 2-Oxindoline-7-carbohydrazide (1-11)

Prepared from methyl oxindole-7-carboxylate in analogy to the general procedure of Example 1. LC-MS (APCI neg): 190 (M−H⁺).

### 1-Hydroxy-2-naphthohydrazide (1-12)

Prepared from methyl 1-hydroxy-2-naphthoate in analogy to the general procedure of Example 1. Structure was confirmed by ¹H NMR (DMSO-d₆).

### 3-Hydroxy-2-naphthohydrazide (1-13)

Prepared from methyl 3-hydroxy-2-naphthoate in analogy to the general procedure of Example 1. Structure was confirmed by ¹H NMR (DMSO-d₆).

### 2-Hydroxy-1-naphthohydrazide (1-14)

Prepared from methyl 2-hydroxy-1-naphthoate in analogy to the general procedure of Example 1. LC-MS (APCI neg): 201.0 (M−H⁺).

### Example 2

### N-(2-(2-(1,1,1-trifluoro-4-oxo-4-(thiophen-2-yl)butan-2-ylidene)hydrazinecarbonyl)phenyl)methanesulfonamide (5)

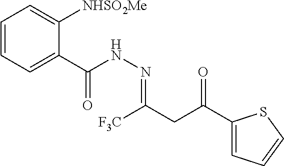

### Step A—Preparation of Methyl 2-(methylsulfonamido)benzoate (2-3)

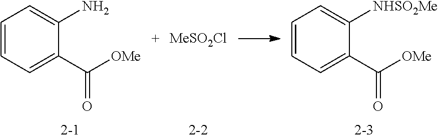

A solution of methane sulfonyl chloride, 2-2, (0.27 mL, 2.2 mmol) in chloroform (1.2 mL) was added dropwise over three minutes to a solution of methyl 2-aminobenzoate, 2-1, (0.26 mL, 2 mmol) in pyridine (0.29 mL) and chloroform (2.6 mL). After stirring at RT for 30 minutes, the mixture was concentrated in vacuo to provide a solid which was washed with hexane to give the product, 2-3, as a rose colored solid (393 mg, 86%). LC-MS (APCI neg): 228.0 (M−H⁺).

### Step B—Preparation of N-(2-(hydrazinecarbonyl)phenyl)methanesulfonamide (9)

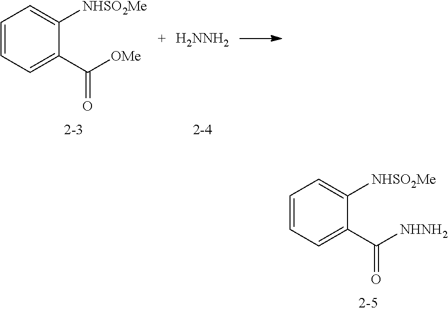

A solution of 1-3 (380 mg, 1.66 mmol) and hydrazine hydrate (0.26 mL, 6.3 mmol) in 2-methoxy ethanol (1 mL) was heated at reflux. After 30 minutes, the mixture was cooled and concentrated in vacuo to afford a transparent oil. The compound was crystallized from ethyl acetate. The solid was filtered and washed with ethyl acetate and ether and air dried to affored the product, 2-5, as a white solid (259 mg, 68%). LC-MS (APCI neg): 228.0 (M−H⁺).

### Step C—Preparation of N-(2-(2-(1,1,1-trifluoro-4-oxo-4-(thiophen-2-yl)butan-2-ylidene)hydrazinecarbonyl)phenyl)methanesulfonamide (5)

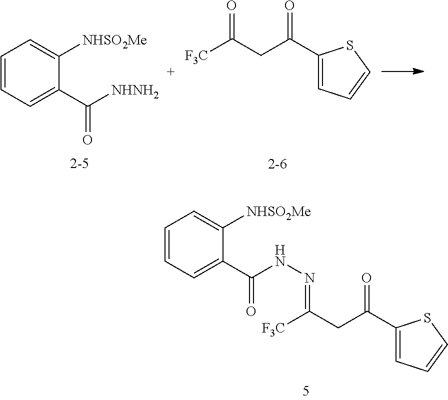

The title compound was prepared from 2-5 (23 mg, 0.10 mmol) and thenoyl trifluoroacetone, 2-6, (Aldrich, 24.4 mg, 0.11 mmol) in DMSO (0.3 mL) and AcOH (1 drop) at 60° C. for 8 hours. The reaction mixture was cooled to room temperature, and water was added dropwise over 1-2 minutes whereupon a thick precipitate formed. The precipitates were collected by vacuum filtration, washed with water, EtOH, and ether and dried in vacuo to afford Compound 5 (30 mg, 70%) as a yellow solid. LC-MS (APCI neg): 432.0 (M−H+).

### Example 3

### 2-Hydroxy-N′-(1,1,1-trifluoro-4-(furan-2-yl)-4-oxobutan-2-ylidene)benzohydrazide (1)

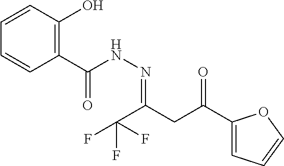

The title compound was prepared from 2-hydroxybenzhydrazide (Alfa, 173 mg, 0.84 mmol), 4,4,4-trifluoro-1-(2-furyl)-1,3-butanedione (Aldrich, 122 mg, 0.8 mmol) and ethanol (1.6 mL) at 90° C. for 8 hours. The reaction mixture was cooled to RT, EtOH (0.5 mL) was added, followed by filtration, triturating with EtOH (3×0.5 mL) and air-drying to afford a residue which was dissolved in DCM (3 mL), loaded on SiO₂ (Flash grade), eluted with 15% ethyl acetate in hexane to afford Compound 1 (20 mg, 7%) as an off-white solid. LC-MS (APCI neg): 339.0 (M−H⁺), 92% purity.

### Example 4

### N′-(4-(benzo[b]thiophen-2-yl)-1,1,1-trifluoro-4-oxobutan-2-ylidene)-2-hydroxybenzohydrazide (8)

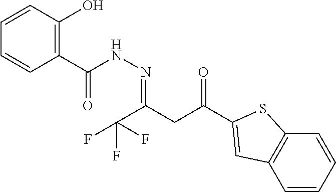

### Step A—Preparation of 1-Benzo[b]thiophen-2-yl-4,4,4-trifluoro-butane-1,3-dione (4-3)

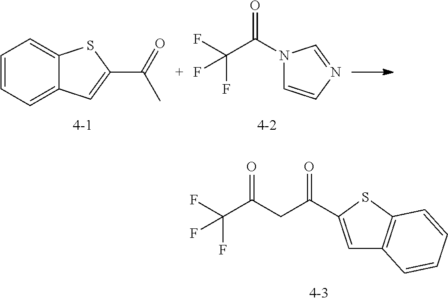

A 50 mL one-neck flask, equipped with magnetic stir bar was charged with 2-acetylbenzo[b]thiophene, 4-1 (Alfa, 2 mmol, 352 mg) and anhydrous THF (Aldrich, 10 mL). The solution was cooled under nitrogen to −78° C. (dry ice-acetone). LiHMDS (Aldrich, 1M solution in THF, 2.2 mmol, 2.2 mL, 1.1 equiv) was added dropwise over 2 min and the solution was stirred for 30 min at −78° C. N-Trifluoroacetylimidazole, 4-2 (Alfa, 2.6 mmol, 0.3 mL, 1.3 equiv) was added over ˜30 sec and the reaction was further stirred at −78° C. for 30 min and then 30 min at 0° C. 2% HCl (−10 mL) was added to the reaction mixture until pH=5. The resulting mixture was poured to a solution of ether-water (30 mL, 1:1). The organic layer was separated, washed with brine (15 mL), dried (MgSO₄) to afford a slowly crystallizing orange oil. This oil was dissolved in DCM-hexane, 1:1 (2 mL), loaded on SiO₂ (Flash, wet in EA-hexane, 1:1), eluted with EA-hexane, 1:6 to afford 4-3 (102 mg, 19%) as a brown solid.

### Step B—N′-(4-(benzo[b]thiophen-2-yl)-1,1,1-trifluoro-4-oxobutan-2-ylidene)-2-hydroxybenzohydrazide (8)

The title compound was prepared in analogy to Example 3 from 2-hydroxybenzhydrazide and 4-3 to afford Compound 8 (19 mg, 31%) as a greenish solid. LC-MS (APCI pos): 407.0 (M+H⁺).

### Example 5

### 2-Hydroxy-N′-(1,1,1-trifluoro-4-oxo-4-(thiophen-2-yl)butan-2-ylidene)benzohydrazide (2)

The title non-proprietary compound was prepared in analogy to Example 3. LC-MS (APCI pos): 357.0 (M+H⁺).

### 2-Hydroxy-N′-(1,1,1-trifluoro-5,5-dimethyl-4-oxohexan-2-ylidene)benzohydrazide (7)

The title compound was prepared in analogy to Example 3. LC-MS (APCI neg): 329.1 (M−H⁺).

### 2-Hydroxy-N′-(1,1,1-trifluoro-4-(1-methyl-1H-pyrazol-5-yl)-4-oxobutan-2-ylidene)benzohydrazide (9)

The title compound was prepared in analogy to Example 3. LC-MS (APCI pos): 353.5 (M−H⁺).

### 3-Chloro-6-fluoro-N′-(1,1,1-trifluoro-4-(5-methylthiophen-2-yl)-4-oxobutan-2-ylidene)benzo[b]thiophene-2-carbohydrazide (10)

The title compound was prepared in analogy to Example 3. LC-MS (APCI pos): 463 (M+H⁺).

### 4-Methyl-N′-(1,1,1-trifluoro-4-oxo-4-(thiophen-2-yl)butan-2-ylidene)-1,2,3-thiadiazole-5-carbohydrazide (11)

The title commercially available compound was prepared in analogy to Example 3. LC-MS (APCI pos): 361 (M+H⁺).

### 3-Chloro-6-fluoro-N′-(1,1,1-trifluoro-5,5-dimethyl-4-oxohexan-2-ylidene)benzo[b]thiophene-2-carbohydrazide (12)

The title compound was prepared in analogy to Example 3. LC-MS (APCI neg): 421 (M−H⁺).

### 2-Hydroxy-N′-(1,1,1-trifluoro-4-(5-methylthiophen-2-yl)-4-oxobutan-2-ylidene)benzohydrazide (13)

The title compound was prepared in analogy to Example 3. LC-MS (APCI pos): 369 (M−H⁺).

### 3-Chloro-N′-(1,1,1-trifluoro-4-oxo-4-(thiophen-2-yl)butan-2-ylidene)benzo[b]thiophene-2-carbohydrazide (14)

The title compound was prepared in analogy to Example 3. LC-MS (APCI neg): 428.9 (M−H⁺).

### 3-Chloro-N′-(1,1,1-trifluoro-4-(furan-2-yl)-4-oxobutan-2-ylidene)benzo[b]thiophene-2-carbohydrazide (15)

The title compound was prepared in analogy to Example 3. LC-MS (ESI neg): 413.5 (M−H⁺).

### 4-Chloro-N-(2-(2-(1,1,1-trifluoro-4-oxo-4-(thiophen-2-yl)butan-2-ylidene)hydrazinecarbonyl)phenyl)benzenesulfonamide (16)

The title compound was prepared in analogy to Example 3. LC-MS (APCI neg): 528.0 (M−H⁺).

### N′-(4-(benzo[b]thiophen-2-yl)-1,1,1-trifluoro-4-oxobutan-2-ylidene)-3-chloro-6-fluorobenzo[b]thiophene-2-carbohydrazide (17)

The title compound was prepared in analogy to Example 3. LC-MS (APCI pos): 499 (M+H⁺).

### N′-(4-(5-chlorothiophen-2-yl)-1,1,1-trifluoro-4-oxobutan-2-ylidene)-2-hydroxybenzohydrazide (19)

The title compound was prepared in analogy to Example 3. LC-MS (APCI pos): 389 (M−H⁺).

### 5-Chloro-2-hydroxy-N′-(1,1,1-trifluoro-4-oxo-4-(thiophen-2-yl)butan-2-ylidene)benzohydrazide (20)

The title compound was prepared in analogy to Example 3. LC-MS (APCI neg): 389 (M−H⁺).

### 3-Chloro-4-methyl-N′-(1,1,1-trifluoro-4-oxo-4-(thiophen-2-yl)butan-2-ylidene)thiophene-2-carbohydrazide (21)

The title compound was prepared in analogy to Example 3. LC-MS (APCI neg): 392.9 (M−H⁺).

### N′-(1,1,1-trifluoro-4-oxo-4-(thiophen-2-yl)butan-2-ylidene)-1H-indole-7-carbohydrazide (22)

The title compound was prepared in analogy to Example 3. LC-MS (APCI neg): 378 (M−H⁺).

### 3-Chloro-N′-(4-(5-chlorothiophen-2-yl)-1,1,1-trifluoro-4-oxobutan-2-ylidene)-6-fluorobenzo[b]thiophene-2-carbohydrazide (23)

The title compound was prepared in analogy to Example 3. LC-MS (APCI neg): 480.9 (M−H⁺).

### 3-Chloro-6-fluoro-N′-(1,1,1-trifluoro-4-oxo-4-(thiophen-2-yl)butan-2-ylidene)benzo[b]thiophene-2-carbohydrazide (24)

The title compound was prepared in analogy to Example 3. LC-MS (APCI neg): 446.7/448.7 (M−H⁺).

### 2-Hydroxy-3-methyl-N′-(1,1,1-trifluoro-4-oxo-4-(thiophen-2-yl)butan-2-ylidene)benzohydrazide (25)

The title compound was prepared in analogy to Example 3. LC-MS (APCI neg): 369 (M−H⁺).

### 4-Nitro-N′-(1,1,1-trifluoro-4-oxo-4-(thiophen-2-yl)butan-2-ylidene)benzohydrazide (26)

The title compound was prepared in analogy to Example 3. LC-MS (APCI neg): 384.0 (M).

### 4-Bromo-N′-(1,1,1-trifluoro-4-oxo-4-(thiophen-2-yl)butan-2-ylidene)benzohydrazide

(27)

The title commerically available compound was prepared in analogy to Example 3. LC-MS (ESI neg): 419.2 (M−H⁺).

### N′-(1,1,1-trifluoro-4-oxo-4-(thiophen-2-yl)butan-2-ylidene)benzo[b]thiophene-2-carbohydrazide (28)

The title compound was prepared in analogy to Example 3. LC-MS (APCI neg) 395 (M−H⁺).

### 3-Chloro-N′-(1,1,1-trifluoro-4-oxo-4-(thiophen-2-yl)butan-2-ylidene)-1H-indole-2-carbohydrazide (30)

The title compound was prepared in analogy to Example 3. LC-MS (APCI neg): 412.0 (M−H⁺).

### 3-Chloro-6-fluoro-N′-(1,1,1-trifluoro-4-(furan-2-yl)-4-oxobutan-2-ylidene)benzo[b]thiophene-2-carbohydrazide (31)

The title compound was prepared in analogy to Example 3. LC-MS (ESI neg): 431.4 (M−H⁺).

### 3-(2-(2-Hydroxybenzoyl)hydrazono)butanamide (32)

The title compound was prepared in analogy to Example 3 with the modification of using refluxing isopropyl alcohol instead of EtOH. LC-MS (ESI neg): 234.1 (M−H⁺).

### 1-Phenyl-N′-(1,1,1-trifluoro-4-oxo-4-(thiophen-2-yl)butan-2-ylidene)-5-(trifluoromethyl)-1H-pyrazole-4-carbohydrazide (34)

The title compound was prepared in analogy to Example 3. LC-MS (APCI neg): 473 (M−H⁺).

### 2,6-Difluoro-N′-(1,1,1-trifluoro-4-oxo-4-(thiophen-2-yl)butan-2-ylidene)benzohydrazide (35)

The title compound was prepared in analogy to Example 3. LC-MS (APCI neg): 375 (M−H⁺).

### Example 6

### 3-Chloro-6-fluoro-N′-(4-(furan-2-yl)-4-oxobutan-2-ylidene)benzo[b]thiophene-2-carbohydrazide (4)

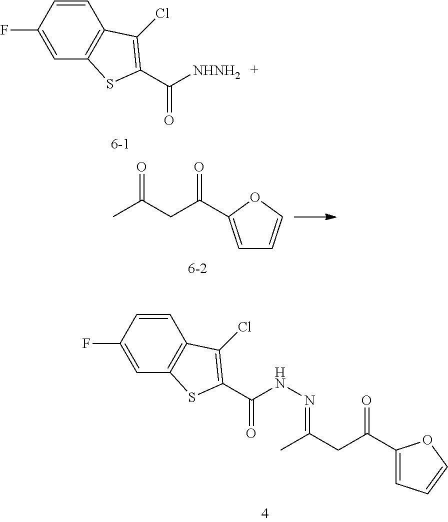

A mixture of the 1-(2-furyl)-1,3-butanedione 6-2 (Alfa, 33 mg, 0.22 mmol), 2 3-chloro-6-fluorobenzo[b]thiophene-2-carbohydrazide 6-1 (Oakwood, 49 mg, 0.2 mmol) in THF (1 to 2 mL) was shaken at 25° C. for 48 hours. The compound was precipitated with hexanes to obtain Compound 4 as a yellow solid (41 mg, 54%). LC-MS (APCI pos): 379.0 (M+H⁺).

### Example 7

### 1-(4-Benzylpiperazin-1-yl)-4,4,4-trifluorobutane-1,3-dione (7-3)

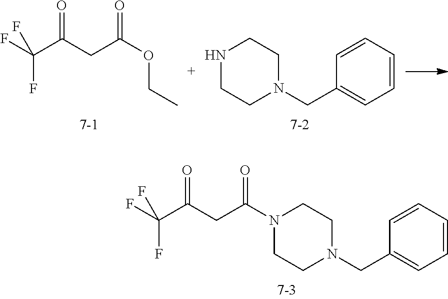

A mixture of ethyl 4,4,4-trifluoroacetate, 7-1, (Aldrich, 2.1 g, 11.3 mmol) and 1-benzylpiperazine, 7-2, (Aldrich, 2 g, 11.3 mmol) in toluene (2.5 mL) was shaken for 8 hours at 90° C. and 105° C. for 8 hours. The solvent was removed in vacuo to obtain the crude mixture. The crude mixture was purified by silica gel flash chromatography to obtain the product (7-3, 2.25 g, 63%). LC-MS (APCI pos): 315 (M+H). Analogous ketoamides may be prepared in a similar fashion.

### Example 8

### N′-(4-(4-benzylpiperazin-1-yl)-1,1,1-trifluoro-4-oxobutan-2-ylidene)-2-hydroxybenzohydrazide (3)

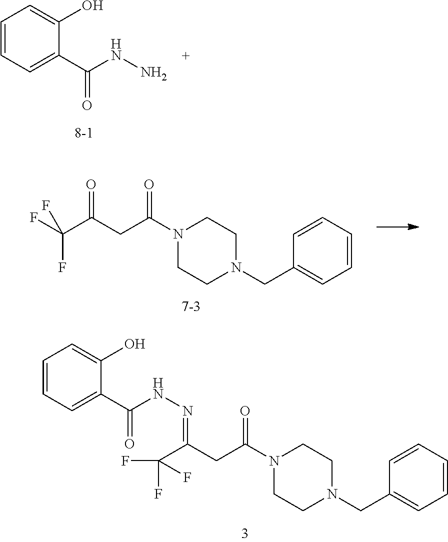

A mixture of dione 7-3 (100 mg, 0.32 mmol) and 2-hydroxybenzhydrazide (8-1, 48 mg, 0.32 mmol) in DMSO (1 mL) and AcOH (15 mg) was shaken at 90° C. for overnight. The crude mixture was purified by silica gel flash chromatography to obtain the Compound 3 (18 mg, 13%): LC-MS (APCI pos): 449.1 (M+H⁺).

### Example 9

### 2-Hydroxy-N′-(1,1,1-trifluoro-4-(4-methylpiperazin-1-yl)-4-oxobutan-2-ylidene)benzohydrazide (6)

The title compound was prepared in analogy to Example 8. LC-MS (APCI pos): 373.3 (M+H⁺).

### 2-Hydroxy-N′-(1,1,1-trifluoro-4-morpholino-4-oxobutan-2-ylidene)benzohydrazide (18)

The title compound was prepared in analogy to Example 8. LC-MS (APCI pos): 360.1 (M+H⁺).

### 2-Hydroxy-N′-(4-oxo-4-(4-phenylpiperidin-1-yl)butan-2-ylidene)benzohydrazide (29)

The title compound was prepared in analogy to Example 8. LC-MS (APCI neg): 378.1 (M−H⁺).

### Example 10

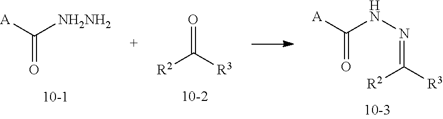

General procedure A. A vial (8-20 mL) equipped with magnetic stir bar, was charged with hydrazides 10-1 (0.30-4.5 mmol, 1 eq.), aldehydes or ketones 10-2 (1.05-1.1 eq.) and solvent (2.3 vol % of AcOH in DMSO; 2 mL/1 mmol of 10-1). The vials were sealed and reaction mixtures were magnetically stirred at RT until TLC (EA-hexane, 1:2) indicated the complete disappearance of starting materials 10-1. Brine was added dropwise over 1-2 min and the reaction mixtures were stirred for 5-10 min. The resulting precipitates were collected by vacuum filtration, washed with water, hexane, hexane-ether, 1:1, ether and air-dried to afford Compounds 10-3.

General procedure B. A vial (8 mL) equipped with magnetic stir bar, was charged with hydrazides 10-1 (0.40 mmol, 1 eq.), aldehydes or ketones 10-2 (1.05 eq.) and solvent (2.3 vol % of AcOH in DMSO or DMSO; 2 mL/1 mmol of 2-1). The vials were sealed and reaction mixtures were magnetically stirred at RT until TLC (EA-hexane, 1:2) indicated the complete disappearance of starting materials 10-1. The reaction was quenched by dropwise addition of a few drops of sat NaHCO₃ or water over 1-2 min. After reaction mixtures were stirred for 5-15 min, the resulting precipitates were collected by vacuum filtration, washed with water, hexane, hexane-ether, 1:1 or ether and air-dried to afford Compounds 10-3.

### Example 11

### 2-Hydroxy-N′-(2-hydroxy-5-methoxybenzylidene)-4-(1H-pyrrol-1-yl)benzohydrazide (36)

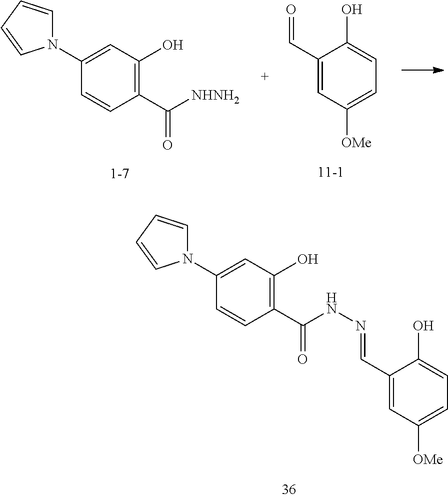

In analogy to general procedure A of Example 10, the title compound was prepared from 2-hydroxybenzhydrazide 1-7 (65 mg, 0.30 mmol), 2-hydroxy-5-methoxybenzaldehyde, 3-2 (Aldrich, 50 mg, 0.33 mmol) and solvent (2.3 vol % of AcOH in DMSO, 0.6 mL). The vial was sealed and reaction mixture was magnetically stirred at RT for 1.5 h until TLC (EA-hexane, 1:2) indicated the complete disappearance of starting material 3-1. Brine was added dropwise over 1-2 min and the reaction mixture was stirred for 10 min. The resulting precipitate was collected by vacuum filtration, washed with water, hexane, ether and air-dried to afford Compound 36 (96 mg, 91%). LC-MS (APCI pos): 352.1 (M+H⁺).

### Example 12

### 3-Hydroxy-N′-(2-hydroxy-5-methoxybenzylidene)-2-naphthohydrazide (37)

The title non-proprietary compound was prepared in analogy to general procedure A of Example 10. LC-MS (APCI pos): 337.1 (M+H⁺).

### 1-Hydroxy-N′-(2-hydroxy-5-methoxybenzylidene)-2-naphthohydrazide (38)

The title compound was prepared in analogy to general procedure A of Example 10. LC-MS (APCI pos): 337.1 (M+H⁺).

### N′-(3-chloro-5-fluoro-2-hydroxybenzylidene)-2-hydroxybenzohydrazide (40)

The title compound was prepared in analogy to general procedure A of Example 10. LC-MS (APCI pos): 309 (M+H⁺).

### N′-(2,6-dihydroxybenzylidene)-2-hydroxybenzohydrazide (41)

The title compound was prepared in analogy to general procedure A of Example 10. LC-MS (APCI pos): 273.0 (M+H⁺).

### N′-(3,5-dibromo-2-hydroxybenzylidene)-2-hydroxybenzohydrazide (42)

The title non-proprietary compound was prepared in analogy to general procedure A of Example 10. LC-MS (APCI pos): 414.9 (M+H⁺).

### 3-Chloro-N′-(2-hydroxy-5-methoxybenzylidene)-1H-indole-2-carbohydrazide (44)

The title compound was prepared in analogy to general procedure A of Example 10. LC-MS (APCI pos): 344.0 (M+H⁺).

### N′-(2-bromo-6-hydroxybenzylidene)-2-hydroxybenzohydrazide (45)

The title compound was prepared in analogy to general procedure A of Example 10. LC-MS (APCI pos): 335.2 (M+H⁺).

### N′-(3-chloro-5-cyclohexyl-2-hydroxybenzylidene)-2-hydroxybenzohydrazide (46)

The title compound was prepared in analogy to general procedure A of Example 10. LC-MS (APCI neg): 371.1 (M−H⁺).

### N′-(5-tert-butyl-2-hydroxybenzylidene)-2-hydroxybenzohydrazide (47)

The title compound was prepared in analogy to general procedure A of Example 10 with additional heating at 60° C. for 24 h. LC-MS (APCI pos): 313.1 (M+H⁺).

### N′-(5-bromo-2-hydroxybenzylidene)-2-hydroxybenzohydrazide (50)

The title non-proprietary compound was prepared in analogy to general procedure A of Example 10. LC-MS (APCI pos): 337.0/338.0 (M+H⁺).

### 4-Chloro-2-hydroxy-N′-(2-hydroxy-5-methoxybenzylidene)benzohydrazide (51)

The title compound was prepared in analogy to general procedure A of Example 10. LC-MS (APCI neg): 319 (M−H⁺).

### N′-(2-hydroxy-5-methoxybenzylidene)-2-oxoindoline-7-carbohydrazide (52)

The title compound was prepared in analogy to general procedure A of Example 10. LC-MS (APCI neg): 324.1 (M−H⁺).

### 2-Hydroxy-N′-(2-hydroxy-6-methoxybenzylidene)benzohydrazide (55)

The title compound was prepared in analogy to general procedure A of Example 10. LC-MS (APCI pos): 287.1 (M+H⁺).

### 2-Hydroxy-N′-(2-hydroxy-5-(trifluoromethoxy)benzylidene)benzohydrazide (57)

The title compound was prepared in analogy to general procedure A of Example 10. LC-MS (APCI neg): 339 (M−H⁺).

### N′-(2-hydroxy-5-methoxybenzylidene)-1H-indole-7-carbohydrazide (58)

The title compound was prepared in analogy to general procedure A of Example 10. LC-MS (APCI pos): 310.1 (M+H⁺).

### N′-(5-chloro-2-hydroxybenzylidene)-2-hydroxybenzohydrazide (59)

The title non-proprietary compound was prepared in analogy to general procedure A of Example 10. LC-MS (APCI pos): 291.0 (M+H⁺).

### 3-Chloro-6-fluoro-N′((2-hydroxynaphthalen-1-yl)methylene)benzo[b]thiophene-2-carbohydrazide (61)

The title compound was prepared in analogy to general procedure A of Example 10. LC-MS (APCI pos): 399 (M+H⁺).

### 2-Hydroxy-N′-(2-hydroxy-5-methoxybenzylidene)-3-methylbenzohydrazide (63)

The title compound was prepared in analogy to general procedure A of Example 10. LC-MS (APCI pos): 301.1 (M+H⁺).

### 2-Hydroxy-N′-(2-hydroxy-5-methoxybenzylidene)-1-naphthohydrazide (66)

The title compound was prepared in analogy to general procedure A of Example 10. LC-MS (APCI pos): 337.1 (M+H⁺).

### 4-Fluoro-2-hydroxy-N′-(2-hydroxy-5-methoxybenzylidene)benzohydrazide (67)

The title compound was prepared in analogy to general procedure A of Example 10. LC-MS (APCI pos): 305 (M+H⁺).

### 2-Hydroxy-N′-(2-hydroxy-5-methylbenzylidene)benzohydrazide (68)

The title non-proprietary compound was prepared in analogy to general procedure A of Example 10. LC-MS (APCI pos): 271.1 (M+H⁺).

### N′-(3,5-dichloro-2-hydroxybenzylidene)-2-hydroxybenzohydrazide (69)

The title commercially available compound was prepared in analogy to general procedure A of Example 10. LC-MS (APCI pos): 325.0 (M+H⁺).

### 3-Chloro-N′-(2-hydroxy-5-methoxybenzylidene)-4-methylthiophene-2-carbohydrazide (70)

The title compound was prepared in analogy to general procedure A of Example 10. LC-MS (APCI pos) 325.0 (M+H⁺).

### 2-Hydroxy-N′-((1-hydroxynaphthalen-2-yl)methylene)benzohydrazide (72)

The title non-proprietary compound was prepared in analogy to general procedure A of Example 10. LC-MS (APCI pos) 307.1 (M+H⁺).

### 2-Hydroxy-N′-(2-hydroxy-5-methoxybenzylidene)-5-methylbenzohydrazide (73)

The title compound was prepared in analogy to general procedure A of Example 10. LC-MS (APCI pos): 301.1 (M+H⁺).

### N′-(3-fluoro-2-hydroxybenzylidene)-2-hydroxybenzohydrazide (74)

The title compound was prepared in analogy to general procedure A of Example 10. LC-MS (APCI pos): 275.1 (M+H⁺).

### 5-Fluoro-2-hydroxy-N′-(2-hydroxy-5-methoxybenzylidene)benzohydrazide (75)

The title compound was prepared in analogy to general procedure A of Example 10. LC-MS (APCI pos): 305 (M+H⁺).

### 2-Hydroxy-N′-(2-hydroxy-5-methoxybenzylidene)-4-methoxybenzohydrazide (76)

The title non-proprietary compound was prepared in analogy to general procedure A of Example 10. LC-MS (APCI neg) 315.1 (M−H⁺).

### Ethyl 6-bromo-5-hydroxy-4-((2-(2-hydroxybenzoyl)hydrazono)methyl)-2-methylbenzofuran-3-carboxylate (77)

The title compound was prepared in analogy to general procedure A of Example 10. LC-MS (APCI pos): 461/463 (M+H⁺).

### 3-(Benzo[d][1,3]dioxol-5-yl)-N′-(2-hydroxy-5-methoxybenzylidene)-1H-pyrazole-5-carbohydrazide (78)

The title compound was prepared in analogy to general procedure A of Example 10. LC-MS (APCI pos): 381.1 (M+H⁺).

### 2-Hydroxy-N′-(2-hydroxy-4-methoxybenzylidene)benzohydrazide (81)

The title compound was prepared in analogy to general procedure A of Example 10. LC-MS (APCI pos): 287.1 (M+H⁺).

### 2-Hydroxy-N′-(2-hydroxy-5-methoxybenzylidene)-5-methoxybenzohydrazide (82)

The title compound was prepared in analogy to general procedure A of Example 10. LC-MS (APCI pos): 317.1 (M+H⁺).

### 2-Hydroxy-N′-(2-hydroxy-5-methoxybenzylidene)benzohydrazide (83)

The title compound was prepared in analogy to general procedure A of Example 10. LC-MS (APCI pos): 287.1 (M+H⁺).

### 5-Chloro-2-hydroxy-N′-(2-hydroxy-5-methoxybenzylidene)benzohydrazide (84)

The title non-proprietary compound was prepared in analogy to general procedure A of Example 10. LC-MS (APCI pos): 321.1 (M+H⁺).

### N′-(5-ethoxy-2-hydroxybenzylidene)-2-hydroxybenzohydrazide (85)

The title compound was prepared in analogy to general procedure A of Example 10. LC-MS (APCI neg): 299.1 (M−H⁺).

### N′-(2,3-dihydroxybenzylidene)-2-hydroxybenzohydrazide (86)

The title compound was prepared in analogy to general procedure A of Example 10. LC-MS (APCI pos): 273.1 (M+H⁺).

### 2-Hydroxy-N′-((3-hydroxy-5-nitrobenzofuran-2-yl)methylene)benzohydrazide (88)

The title compound was prepared in analogy to general procedure A of Example 10.

### 2-Hydroxy-N′-((2-hydroxynaphthalen-1-yl)methylene)benzohydrazide (89)

The title commercially available compound was prepared in analogy to general procedure A of Example 10. LC-MS (APCI pos): 307.1 (M+H⁺).

### N′-(2,4-dihydroxybenzylidene)-2-hydroxybenzohydrazide (90)

The title compound was prepared in analogy to general procedure A of Example 10. LC-MS (APCI pos): 273.0 (M+H⁺).

### N′-(5-chloro-2-hydroxy-3-methoxybenzylidene)-2-hydroxybenzohydrazide (91)

The title compound was prepared in analogy to general procedure A of Example 10. LC-MS (APCI pos): 321 (M+H⁺).

### 3-Chloro-N′-(5-chloro-2-hydroxybenzylidene)-4-methylthiophene-2-carbohydrazide (92)

The title compound was prepared in analogy to general procedure A of Example 10. LC-MS (APCI pos): 328.9 (M+H⁺).

### 2-Amino-N′-(2-amino-5-chlorobenzylidene)benzohydrazide (93)

The title compound was prepared in analogy to general procedure A of Example 10. LC-MS (APCI pos): 289.0 (M+H⁺).

### 2-Hydroxy-N′-(2-hydroxy-3-methoxybenzylidene)benzohydrazide (94)

The title compound was prepared in analogy to general procedure A of Example 10. LC-MS (APCI pos): 287.1 (M+H⁺).

### 2-Fluoro-6-hydroxy-N′-(2-hydroxy-5-methoxybenzylidene)benzohydrazide (95)

The title compound was prepared in analogy to general procedure A of Example 10. LC-MS (APCI pos): 305.1 (M+H⁺).

### 2-Hydroxy-N′-(2-hydroxy-3-methylbenzylidene)benzohydrazide (96)

The title compound was prepared in analogy to general procedure A of Example 10. LC-MS (APCI pos): 271.1 (M+H⁺).

### N′-((2-hydroxynaphthalen-1-yl)methylene)-3-methyl-1H-pyrazole-5-carbohydrazide (97)

The title compound was prepared in analogy to general procedure A of Example 10. LC-MS (APCI pos): 295.1 (M+H⁺).

### 5-Bromo-2-hydroxy-N′-(2-hydroxy-5-methoxybenzylidene)benzohydrazide (98)

The title compound was prepared in analogy to general procedure A of Example 10. LC-MS (APCI pos): 365 (M+H⁺).

### 2-Hydroxy-N′-(2-hydroxy-5-nitrobenzylidene)benzohydrazide (99)

The title compound was prepared in analogy to general procedure A of Example 10. LC-MS (APCI neg): 300 (M−H⁺).

### N-(2-(2-(2-hydroxy-5-methoxybenzylidene)hydrazinecarbonyl)phenyl)methanesulfonamide (100)

The title compound was prepared in analogy to general procedure A of Example 10. LC-MS (APCI pos): 364.0 (M+H⁺).

### N′-(3,5-difluoro-2-hydroxybenzylidene)-2-hydroxybenzohydrazide (101)

The title compound was prepared in analogy to general procedure A of Example 10. LC-MS (APCI pos): 293 (M+H⁺).

### N′-(1-(5-chloro-2-hydroxyphenyl)-2,2,2-trifluoroethylidene)-2-hydroxybenzohydrazide (102)

The title compound was prepared in analogy to general procedure A of Example 10. LC-MS (APCI neg): 357.0 (M−H⁺).

### 2-Hydroxy-N′-(2-hydroxy-5-methoxybenzylidene)-5-nitrobenzohydrazide (104)

The title compound was prepared in analogy to general procedure A of Example 10. LC-MS (APCI neg): 330 (M−H⁺).

### 8-Hydroxy-N′-(2-hydroxy-5-methoxybenzylidene)-1-naphthohydrazide (105)

The title compound was prepared in analogy to general procedure A of Example 10. LC-MS (APCI neg): 335.1 (M−H⁺).

### N′-(3-ethoxy-2-hydroxybenzylidene)-2-hydroxybenzohydrazide (106)

The title compound was prepared in analogy to general procedure A of Example 10. LC-MS (APCI pos): 301.1 (M+H⁺).

### 3-(5-Chlorothiophen-2-yl)-N′-(2-hydroxy-5-methoxybenzylidene)-1H-pyrazole-5-carbohydrazide (107)

The title compound was prepared in analogy to general procedure A of Example 10. LC-MS (APCI pos): 377.0 (M+H⁺).

### N′-(3-bromo-5-chloro-2-hydroxybenzylidene)-2-hydroxybenzohydrazide (108)

The title compound was prepared in analogy to general procedure A of Example 10. LC-MS (APCI pos): 369.9 (M+H⁺).

### N′-(3-bromo-2-hydroxy-5-methoxybenzylidene)-2-hydroxybenzohydrazide (109)

The title compound was prepared in analogy to general procedure A of Example 10. LC-MS (APCI pos): 366.9 (M+H⁺).

### 2-Amino-N′-(2-hydroxy-5-methoxybenzylidene)benzohydrazide (110)

The title compound was prepared in analogy to general procedure A of Example 10. LC-MS (APCI pos): 286.1 (M+H⁺).

### N′-(5-Chloro-2-hydroxybenzylidene)-4-methyl-1,2,3-thiadiazole-5-carbohydrazide (111)

The title compound was prepared in analogy to general procedure A of Example 10. LC-MS (APCI neg): 295 (M−H⁺).

### N-(4-chloro-2-β2-(2-hydroxybenzoyl)hydrazono)methyl)phenyl)-2,4-difluorobenzenesulfonamide (114)

The title compound was prepared in analogy to general procedure A of Example 10. LC-MS (APCI neg): 464 (M−H⁺).

### Example 13

### 2-Hydroxy-N′-((4-hydroxy-3′-methoxybiphenyl-3-yl)methylene)benzohydrazide (48)

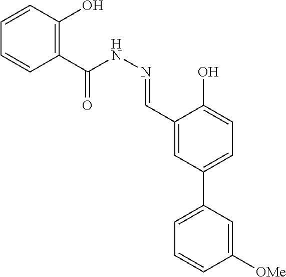

### Step A—Preparation of 4-Hydroxy-3′-methoxybiphenyl-3-carbaldehyde (13-3)

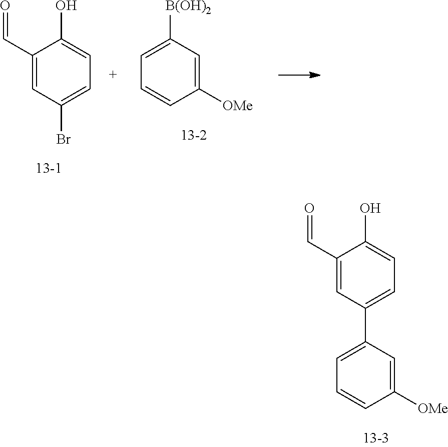

A mixture of 5-bromosalicylaldehyde, 13-1, (Aldrich, 568 mg, 2.8 mmol), 3-methoxyphenyl boronic acid, 13-2, (Aldrich, 472 mg, 3.1 mmol) and a 3:1 mixture of DME and water (4.8 mL) was bubbled with nitrogen for 10 minutes. Sodium carbonate (448 mg, 4.2 mmol) was added to the mixture, followed by Pd(dppf)Cl₂ (Aldrich, 115 mg, 0.14 mmol). Nitrogen was bubbled through the mixture for 5 minutes and the reaction was heated at reflux for 19 hours. (LC-MS (APCI neg) 227.0 (M−H⁺). Water was added and the mixture was shaken and extracted with DCM. The organic fraction was washed with water and brine, dried over MgSO₄, filtered and concentrated. The material was purified by silica gel chromatography to afford 13-3 (189 mg, 29%) as a yellow oil. LC-MS (APCI neg) 227.0 (M−H⁺).

### Step B—2-Hydroxy-N′-((4-hydroxy-3′-methoxybiphenyl-3-yl)methylene)benzohydrazide (48)

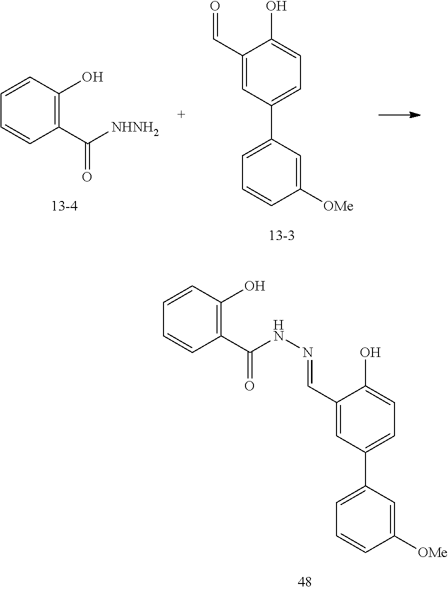

In analogy to general procedure B of Example 10, the title compound was prepared from 2-hydroxybenzhydrazide 13-4 (30 mg, 0.20 mmol), aldehyde, 13-3 (53 mg, 0.21 mmol) and solvent. The vial was sealed and the reaction mixture was magnetically stirred at RT for 30 minutes whereupon a precipitate had 1-2 minutes and the mixture was stirred for 15 minutes. The resulting precipitate was collected by vacuum filtration, washed with water, hexane, hexane-ether, 1:1 and air-dried to afford Compound 48 (69 mg, 95%) as a white solid. LC-MS (APCI pos): 363.1 (M+H⁺)

### Example 14

### 2-((2-(2-Hydroxybenzoyl)hydrazono)methyl)benzamide (64)

The title compound was prepared in analogy to general procedure B of Example 10. LC-MS (APCI neg): 282.1 (M−H⁺).

### N′-(2-amino-5-chlorobenzylidene)-2-hydroxybenzohydrazide (65)

The title compound was prepared in analogy to general procedure B of Example 10. LC-MS (APCI pos): 290.0 (M+H⁺).

### N′-(4-(diethylamino)-2-hydroxybenzylidene)-2-hydroxybenzohydrazide (80)

The title compound was prepared in analogy to general procedure B of Example 10. LC-MS (APCI neg): 326.1 (M−H⁺).

### 2-Hydroxy-N′-(2-hydroxy-4-morpholinobenzylidene)benzohydrazide (87)

The title compound was prepared in analogy to general procedure B of Example 10. LC-MS (APCI pos): 342.1 (M+H⁺).

### N-(2-(2-(2-acetamido-5-chlorobenzylidene)hydrazinecarbonyl)phenyl)acetamide (113)

The title compound was prepared in analogy to general procedure B of Example 10. LC-MS (APCI pos): 373.6 (M+H⁺).

### Example 15

The following compounds were obtained from commercial sources.

| No. | Source |
|---|---|
| 11 | TOSLab (Ekaterinburg, Russia) |
| 27 | ChemDiv (San Diego, CA) |
| 33 | ChemDiv (San Diego, CA) |
| 39 | Chembridge (San Diego, CA) |
| 43 | Aldrich (Milwaukee, WI) |
| 49 | Timtec (Newark, DE) |
| 53 | AnaLogix (Burlington, WI) |
| 54 | AnaLogix (Burlington, WI) |
| 56 | Timtec (Newark, DE) |
| 60 | Timtec (Newark, DE) |
| 62 | Aldrich (Milwaukee, WI) |
| 69 | ChemDiv (San Diego, CA) |
| 71 | Aldrich (Milwaukee, WI) |
| 79 | Chembridge (San Diego, CA) |
| 89 | Chembridge (San Diego, CA) |
| 103 | Chembridge (San Diego, CA) |
| 112 | Chembridge (San Diego, CA) |
| 115 | Aldrich (Milwaukee, WI) |
| 116 | AnaLogix (Burlington, WI) |

### Example 16

### FRET Assay

Fluorescence (or Förster) Resonance Energy Transfer (FRET) is a distance-dependent, non-radiative transfer of energy in which the de-excitation of one fluorophore (donor) is coupled to excitation of another fluorophore (acceptor). FRET occurs if (1) the quantum of energy emitted by a donor fluorophore corresponds to an acceptor fluorophore's excitation energy, (2) the orientations of donor and acceptor transition dipoles are nearly parallel and (3) the donor fluorescent emission spectrum overlaps the acceptor absorption spectrum.

In this assay, assembly of Aβ₁₋₄₂ oligomer formation is monitored by FRET using N-terminal conjugates of fluorescein-Aβ₁₋₄₂ as both the donor and acceptor fluorophore (fluorescein-fluorescein Rₒ˜45 Å). Aβ₁₋₄₂ monomers assembling into oligomeric species results in a decrease of fluorescence as the fluorescein labeled Aβ₁₋₄₂ peptides become proximal to each other and FRET efficiency increases. Inhibition of Aβ₁₋₄₂ assembly is observed as the absence or attenuation of fluorescein quenching.

FRET and FP assays are performed in 384-well Corning Non-Binding Surface, black, opaque microtiter plates, and the assay buffer consists of 25 mM MOPS-Tris (pH 8.0) with 100 mM MgCl₂. The assay volume, containing 0.2 μM FITC-Aβ(1-42) and 0.8 μM Aβ(1-42), is 50 μl and the temperature is 37° C. ADDL assembly is monitored on a Tecan GENios Pro plate reader, exciting at a wavelength of 485 nm and detecting emission at a wavelength of 515 nm. Kinetic traces are collected by recording fluorescence intensity and polarization readings every five minutes over about a three hour time course. Negative control reactions, which do not appreciably assemble into ADDLs during this time, lack MgCl₂ but contain all other buffer and peptide components. Positive control reactions contain all buffer components in the absence of added small molecule or antibody reagents. To test for ADDL assembly inhibition, the compound was incubated with the peptide mixture at six concentrations from about 10 μM decreasing to about 0.03 μM.

Results for the compounds of the invention that were tested in this assay are shown in Table 2 below.

TABLE 2

| No. | IC₅₀ |
|---|---|
| 1 | 4.2 |
| 2 | 4.5 |
| 3 | 5.2 |
| 4 | 5.9 |
| 5 | 6 |
| 6 | 7.1 |
| 7 | 7.4 |
| 8 | 7.7 |
| 9 | 8.2 |
| 10 | 8.3 |
| 11 | 8.4 |
| 12 | 8.6 |
| 13 | 9.1 |
| 14 | 9.5 |
| 15 | 9.5 |
| 16 | 9.7 |
| 17 | 10.2 |
| 18 | 10.4 |
| 19 | 11.5 |
| 20 | 11.9 |
| 21 | 12.5 |
| 22 | 13.5 |
| 23 | 16 |
| 24 | 16.1 |
| 25 | 16.3 |
| 26 | 16.5 |
| 27 | 18.8 |
| 28 | 19.4 |
| 29 | 26.7 |
| 30 | 28.3 |
| 31 | 30.2 |
| 32 | 35 |
| 33 | 40.8 |
| 34 | 40.9 |
| 35 | 41.2 |
| 36 | 1 |
| 37 | 1.2 |
| 38 | 1.2 |
| 39 | 1.6 |
| 40 | 2.1 |
| 41 | 2.3 |
| 42 | 2.8 |
| 43 | 3.2 |
| 44 | 3.2 |
| 45 | 3.3 |
| 46 | 3.3 |
| 47 | 3.5 |
| 48 | 3.7 |
| 49 | 3.8 |
| 50 | 4.1 |
| 51 | 4.2 |
| 52 | 4.2 |
| 53 | 4.6 |
| 54 | 5 |
| 55 | 5.3 |
| 56 | 6.1 |
| 57 | 6.3 |
| 58 | 6.4 |
| 59 | 6.5 |
| 60 | 6.5 |
| 61 | 6.7 |
| 62 | 6.7 |
| 63 | 7 |
| 64 | 7.1 |
| 65 | 7.5 |
| 66 | 7.7 |
| 67 | 8.3 |
| 68 | 9.2 |
| 69 | 9.2 |
| 70 | 9.4 |
| 71 | 9.7 |
| 72 | 10.4 |
| 73 | 10.4 |
| 74 | 10.9 |
| 75 | 10.9 |
| 76 | 11.1 |
| 77 | 11.6 |
| 78 | 11.6 |
| 79 | 13.3 |
| 80 | 14.4 |
| 81 | 15 |
| 82 | 15.1 |
| 83 | 15.4 |
| 84 | 15.5 |
| 85 | 16 |
| 86 | 17.4 |
| 87 | 18.3 |
| 88 | 20.4 |
| 89 | 20.6 |
| 90 | 21.4 |
| 91 | 22.8 |
| 92 | 23.7 |
| 93 | 23.9 |
| 94 | 24.9 |
| 95 | 26.2 |
| 96 | 28.6 |
| 97 | 30.6 |
| 98 | 31.9 |
| 99 | 32.5 |
| 100 | 33.2 |
| 101 | 33.4 |
| 102 | 34 |
| 103 | 34.5 |
| 104 | 35.1 |
| 105 | 36.5 |
| 106 | 39.2 |
| 107 | 40.1 |
| 108 | 41.7 |
| 109 | 42 |
| 110 | 42.3 |
| 111 | 42.9 |
| 112 | 45.4 |
| 113 | 46 |
| 114 | 46.7 |
| 115 | 47.6 |
| 116 | 50 |

### Example 17

### Alternating Lever Cyclic Ratio Rat Model

Preparations of Aβ₁₋₄₂ ADDLs and a potential therapeutic compound under the Alternating Lever Cyclic Ratio (ALCR) rat model of AD were tested to show in vivo efficacy. This highly sensitive model has been able to detect cognitive deficits due to direct injection of cell-derived Aβ oligomers into rat brain. Using this technique, a direct injection of ADDLs made from synthetic Aβ₁₋₄₂ and a putative therapeutic compound under the ALCR procedure were tested.

In this task, rats learned a complex sequence of lever-pressing requirements in order to earn food reinforcement in a two-lever experimental chamber. Subjects alternated between two levers by switching to the other lever after pressing the first lever enough to get food reward. The exact number of presses required for each food reward changed, first increasing from 2 responses per food pellet up to 56 presses per food pellet, then decreasing back to 2 responses per pellet. Intermediate values were based on the quadratic function, x²−x. One cycle was an entire ascending and descending sequence of these lever press requirements (e.g., 2, 6, 12, 20, 30, 42, 56, 56, 42, 30, 20, 12, 6, and 2 presses per food reward). Six such full cycles were presented during each daily session. Errors were scored when the subject perseveres on a lever after pressing enough to get the food reward, i.e., did not alternate (a Perseveration Error), or when a subject switched levers before completing the response requirement on that lever (an Approach Error).

### Materials and Methods

Synthetic Aβ₁₋₄₂ powder was dissolved in 1,1,1,3,3,3-hexafluorisopropanol (HFIP) to afford a solution of Aβ₁₋₄₂ in HFIP of about 1 mM and allowed to incubate at ambient temperature for about 1 h. The resulting solution was chilled on ice for about 5-10 min, then aliquoted into eppendorf tubes to provide about 50 μL of solution per tube. The tubes were then placed in a chemical fume hood and allowed to stand overnight to allow the HFIP to evaporate under a slow stream of nitrogen. To remove final traces of HFIP, the tubes were subjected to two SpeedVac cycles of 15 min at room temperature and about 15 to 25 mm Hg of vacuum. The resulting films of monomerized Aβ₁₋₄₂ peptide were stored over desiccant at −80° C. until used.

A tube of monomerized Aβ₁₋₄₂ peptide was warmed to room temperature and the Aβ₁₋₄₂ peptide was dissolved in anhydrous DMSO to afford a peptide stock DMSO solution containing about 10 μM to about 100 μM Aβ₁₋₄₂ peptide in DMSO.

2 μL of a 20 mM stock solution of test compound (Compound 2 and Compound 89 from Table 1) in anhydrous DMSO was added to an appropriate amount of neural basal media (phenol red free, Gibco 12348-017) to provide the requisite and noted concentration of the test compound in neural basal media. The concentration of the test compound is listed below in Table 3.

For the Aβ₁₋₄₂ peptide only treatment, peptide stock DMSO solution was added to 37° C. neural basal to obtain the requisite Aβ₁₋₄₂ peptide monomer concentration, provided that the maximum concentration of DMSO is 1% or less, and the tube was vortexed for 30 to 60 seconds, spun down briefly in a microfuge and incubated at 37° C. for 15 min prior to the start of injections.

For the Aβ₁₋₄₂ peptide plus test compound treatment, peptide stock DMSO solution was added to 37° C. compound neural basal media solution to obtain the requisite Aβ₁₋₄₂ peptide monomer concentration, provided that the maximum concentration of DMSO is 1% or less, and the tube was vortexed for 30 to 36 seconds, spun down briefly in a microfuge and incubated at 37° C. for 15 min prior to the start of injections.

For control injections, compound neural basal media solution was incubated at 37° C. for 15 min prior to the start of injections.

Rats: Rats are trained under ALCR until their error rates are stable. Once the rats are placed upon the final ALCR procedure, training sessions are conducted 7 days each week until the end of the study.

Surgery: All rats received a single 28 gauge cannula, which was permanently affixed to the skull, and aimed at the lateral ventricle. Half of the rats received cannula in the right ventricle and half receive cannula in the left ventricle. Rats were allowed 5 days to recover from surgery before training resumed.

Injection of Test Material and ALCR Testing: Test were conducted about every fourth day. Animals received a 10-20 μL injection of control, peptide, or peptide plus compound solutions via the implanted cannula over about 3 to 4 minutes. Animals were tested about 3 hours following injection.

Error Rate Analysis: All error rates were compared to baseline error rates consisting of at least 3 non-treatment days temporally contiguous to the injection. Student's T test of statistical inference was used for analysis of effects.

### Results

The preservation errors and approach errors are found in Table 3 below.

TABLE 3

| | | | | 1 nM | | 1 nM | | | |
|---|---|---|---|---|---|---|---|---|---|
| | | | | ADDLs + | | ADDLs + | 1 nM | | |
| | | 27 nM | | 2 μM | | 2 μM | ADDLs + | 2 μM | |
| | | Aβ₁₋₄₂ | 1 nM | Cmpd | 1 nM | Cmpd | 2 μM | Cmpd | 2 μM |
| | Vehicle | Monomer | ADDLs | 89 | ADDLs | 89 | Cmpd 2 | 89 | Cmpd 2 |
| Perseveration | 106% | 117% | 133% | 92% | 127% | 107% | 105% | 112% | 85% |
| Errors | | | | | | | | | |
| Approach | 105% | 105% | 102% | 100% | 142% | 111% | 115% | 101% | 92% |
| Errors | | | | | | | | | |

As can be seen from Table 3, the presence of ADDLs compared to presence of vehicle or Aβ₁₋₄₂ monomer alone, increased the Perseveration error. When ADDLs were injected along with either Compound 2 or Compound 89, the Perseveration error decreased significantly. It is contemplated that addition of one or more of the compounds will not increase or decrease error rates when given alone, but when combined with ADDLs, will eliminate the increase in Perseveration Error. Thus, it is contemplated that at least one or more of the compounds described herein will reduce errors produced by Aβ₁₋₄₂ ADDLs.

From the foregoing description, various modifications and changes in the compositions and methods will occur to those skilled in the art. All such modifications coming within the scope of the appended claims are intended to be included therein.

1. A method for antagonizing neurotoxic ADDL formation from monomeric Aβ₁₋₄₂ by contacting monomeric Aβ₁₋₄₂ with an effective amount of a compound of the formula: 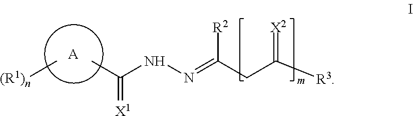
wherein:
A is a 5-10 membered heteroaryl ring having 1 to 3 heteroatoms or an aryl ring;
X¹ and X² are independently selected from the group consisting of oxygen, sulfur or N—OR⁴;
R¹ is selected from the group consisting of hydroxy, halo, nitro, C₁₋₆ alkyl, C₁₋₆ haloalkyl, C₁₋₆ alkoxy, —N(R⁵)(R⁶), C₃₋₁₀ cycloalkyl, aryl, heteroaryl, heterocyclic, wherein the aryl, heteroaryl, and heterocyclic group is optionally substituted with 1-3 R⁹ groups;
R² is selected from the group consisting of hydrogen, C₁₋₆ alkyl, and C₁₋₆ haloalkyl;
R³ is selected from the group consisting of C₁₋₆ alkyl, C₁₋₆ alkoxy, —N(R⁵)(R⁶), and R⁸;
R⁴ is selected from the group consisting of hydrogen and C₁₋₆ alkyl;
each R⁵ is independently selected from the group consisting of hydrogen, C₁₋₆ alkyl, —C(═O)—C₁₋₆ alkyl, and —SOAR);
each R⁶ is independently selected from the group consisting of hydrogen and C₁₋₆ alkyl;
R⁷ is selected from the group consisting of hydrogen, C₁₋₆ alkyl, and aryl optionally substituted with 1 to 3 of C₁₋₄ alkyl or halo;
R⁸ is selected from the group consisting of aryl, biaryl, heteroaryl, and heterocyclic, wherein each R⁸ is optionally substituted with 1-4 R⁹ groups;
each R⁹ is independently selected from the group consisting of hydroxy, halo, nitro, C₁₋₆ alkyl, C₂₋₆ alkenyl, C₁₋₆ alkoxy, C₁₋₆ haloalkoxy, C₃₋₁₀ cycloalkyl, aralkyl, aryl, —N(R⁵)(R⁶), carboxyl, carboxyl ester, and heterocyclic;
n is 0, 1, 2, or 3; and
m is 0 or 1;
or a pharmaceutically acceptable salt, stereoisomer, tautomer, or prodrug thereof;
with the proviso that the compound exhibits an IC₅₀ of about 50 μM or less in the FRET assay.

2. A method of inhibiting, regulating and/or modulating the ADDL-induced neuronal dysfunction and/or neurotoxicity in a neuronal cell or neuronal tissue by inhibiting the formation of ADDLs which method comprises contacting Aβ₁₋₄₂ monomers which may be in the presence of a neuronal cell with an effective amount of a compound of the formula: 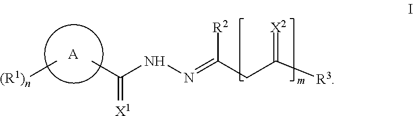
wherein:
A is a 5-10 membered heteroaryl ring having 1 to 3 heteroatoms or an aryl ring;
X¹ and X² are independently selected from the group consisting of oxygen, sulfur or N—OR⁴;
R¹ is selected from the group consisting of hydroxy, halo, nitro, C₁₋₆ alkyl, C₁₋₆ haloalkyl, C₁₋₆ alkoxy, —N(R⁵)(R⁶), C₃₋₁₀ cycloalkyl, aryl, heteroaryl, heterocyclic, wherein the aryl, heteroaryl, and heterocyclic group is optionally substituted with 1-3 R⁹ groups;
R² is selected from the group consisting of hydrogen, C₁₋₆ alkyl, and C₁₋₆ haloalkyl;
R³ is selected from the group consisting of C₁₋₆ alkyl, C₁₋₆ alkoxy, —N(R⁵)(R⁶), and R⁸;
R⁴ is selected from the group consisting of hydrogen and C₁₋₆ alkyl;
each R⁵ is independently selected from the group consisting of hydrogen, C₁₋₆ alkyl, —C(═O)—C₁₋₆ alkyl, and —SO₂(R⁷);
each R⁶ is independently selected from the group consisting of hydrogen and C₁₋₆ alkyl;
R⁷ is selected from the group consisting of hydrogen, C₁₋₆ alkyl, and aryl optionally substituted with 1 to 3 of C₁₋₄ alkyl or halo;
R⁸ is selected from the group consisting of aryl, biaryl, heteroaryl, and heterocyclic, wherein each R⁸ is optionally substituted with 1-4 R⁹ groups;
each R⁹ is independently selected from the group consisting of hydroxy, halo, nitro, C₁₋₆ alkyl, C₂₋₆ alkenyl, C₁₋₆ alkoxy, C₁₋₆ haloalkoxy, C₃₋₁₀ cycloalkyl, aralkyl, aryl, —N(R⁵)(R⁶), carboxyl, carboxyl ester, and heterocyclic;
n is 0, 1, 2, or 3; and
m is 0 or 1;
or a pharmaceutically acceptable salt, stereoisomer, tautomer, or prodrug thereof;
with the proviso that the compound exhibits an IC₅₀ of about 50 μM or less in the FRET assay.

3. A method of inhibiting, regulating and/or modulating formation of amyloid-β oligomers or the activity of said oligomers in a patient suffering from or at risk of suffering from a disease associated with the formation of amyloid-β oligomers, wherein said method comprises administering to said patient a therapeutically effective amount of a compound of the formula: 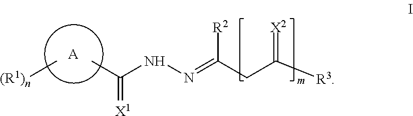
wherein:
A is a 5-10 membered heteroaryl ring having 1 to 3 heteroatoms or an aryl ring;
X¹ and X² are independently selected from the group consisting of oxygen, sulfur or N—OR⁴;
R¹ is selected from the group consisting of hydroxy, halo, nitro, C₁₋₆ alkyl, C₁₋₆ haloalkyl, C₁₋₆ alkoxy, —N(R⁵)(R⁶), C₃₋₁₀ cycloalkyl, aryl, heteroaryl, heterocyclic, wherein the aryl, heteroaryl, and heterocyclic group is optionally substituted with 1-3 R⁹ groups;
R² is selected from the group consisting of hydrogen, C₁₋₆ alkyl, and C₁₋₆ haloalkyl;
R³ is selected from the group consisting of C₁₋₆ alkyl, C₁₋₆alkoxy, —N(R⁵)(R⁶), and R⁸;
R⁴ is selected from the group consisting of hydrogen and C₁₋₆ alkyl;
each R⁵ is independently selected from the group consisting of hydrogen, C₁₋₆ alkyl, —C(═O)—C₁₋₆ alkyl, and —SO₂(R⁷);
each R⁶ is independently selected from the group consisting of hydrogen and C₁₋₆ alkyl;
R⁷ is selected from the group consisting of hydrogen, C₁₋₆ alkyl, and aryl optionally substituted with 1 to 3 of C₁₋₄ alkyl or halo;
R⁸ is selected from the group consisting of aryl, biaryl, heteroaryl, and heterocyclic, wherein each R⁸ is optionally substituted with 1-4 R⁹ groups;
each R⁹ is independently selected from the group consisting of hydroxy, halo, nitro, C₁₋₆ alkyl, C₂₋₆ alkenyl, C₁₋₆ alkoxy, C₁₋₆ haloalkoxy, C₃₋₁₀ cycloalkyl, aralkyl, aryl, —N(R⁵)(R⁶), carboxyl, carboxyl ester, and heterocyclic;
n is 0, 1, 2, or 3; and
m is 0 or 1;
or a pharmaceutically acceptable salt, stereoisomer, tautomer, or prodrug thereof;
with the proviso that the compound exhibits an IC₅₀ of about 50 μM or less in the FRET assay.

4. A method for treating a patient suffering from or at risk of suffering from a disease selected from the group consisting of Alzheimer's disease, Down's Syndrome, stroke, mild cognitive impairment, focal ischemia associated dementia, and neuronal degeneration comprising administering to said patient a therapeutically effective amount of a compound of the formula: 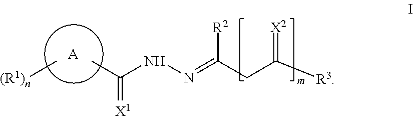
wherein:
A is a 5-10 membered heteroaryl ring having 1 to 3 heteroatoms or an aryl ring;
X¹ and X² are independently selected from the group consisting of oxygen, sulfur or N—OR⁴;
R¹ is selected from the group consisting of hydroxy, halo, nitro, C₁₋₆ alkyl, C₁₋₆ haloalkyl, C₁₋₆ alkoxy, —N(R⁵)(R⁶), C₃₋₁₀ cycloalkyl, aryl, heteroaryl, heterocyclic, wherein the aryl, heteroaryl, and heterocyclic group is optionally substituted with 1-3 R⁹ groups;
R² is selected from the group consisting of hydrogen, C₁₋₆ alkyl, and C₁₋₆ haloalkyl;
R³ is selected from the group consisting of C₁₋₆ alkyl, C₁₋₆ alkoxy, —N(R⁵)(R⁶), and R⁸;
R⁴ is selected from the group consisting of hydrogen and C₁₋₆ alkyl;
each R⁵ is independently selected from the group consisting of hydrogen, C₁₋₆ alkyl, —C(═O)—C₁₋₆ alkyl, and —SO₂(R⁷);
each R⁶ is independently selected from the group consisting of hydrogen and C₁₋₆ alkyl;
R⁷ is selected from the group consisting of hydrogen, C₁₋₆ alkyl, and aryl optionally substituted with 1 to 3 of C₁₋₄ alkyl or halo;
R⁸ is selected from the group consisting of aryl, biaryl, heteroaryl, and heterocyclic, wherein each R⁸ is optionally substituted with 1-4 R⁹ groups;
each R⁹ is independently selected from the group consisting of hydroxy, halo, nitro, C₁₋₆ alkyl, C₂₋₆ alkenyl, C₁₋₆ alkoxy, C₁₋₆ haloalkoxy, C₃₋₁₀ cycloalkyl, aralkyl, aryl, —N(R⁵)(R⁶), carboxyl, carboxyl ester, and heterocyclic;
n is 0, 1, 2, or 3; and
m is 0 or 1;
or a pharmaceutically acceptable salt, stereoisomer, tautomer, or prodrug thereof;
with the proviso that the compound exhibits an IC₅₀ of about 50 μM or less in the FRET assay.

5. A method of enhancing cognitive function in a patient who is suffering from or at risk of suffering from a disease characterized by diminished cognitive function due to ADDL neurotoxicity, which method comprises administering to the patient a therapeutically effective amount of a compound of the formula: 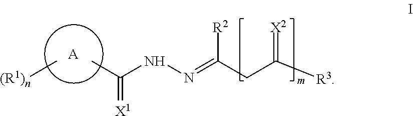
wherein:
A is a 5-10 membered heteroaryl ring having 1 to 3 heteroatoms or an aryl ring;
X¹ and X² are independently selected from the group consisting of oxygen, sulfur or N—OR⁴;
R¹ is selected from the group consisting of hydroxy, halo, nitro, C₁₋₆ alkyl, C₁₋₆ haloalkyl, C₁₋₆ alkoxy, —N(R⁵)(R⁶), C₃₋₁₀ cycloalkyl, aryl, heteroaryl, heterocyclic, wherein the aryl, heteroaryl, and heterocyclic group is optionally substituted with 1-3 R⁹ groups;
R² is selected from the group consisting of hydrogen, C₁₋₆ alkyl, and C₁₋₆ haloalkyl;
R³ is selected from the group consisting of C₁₋₆ alkyl, C₁₋₆ alkoxy, —N(R⁵)(R⁶), and R⁸;
R⁴ is selected from the group consisting of hydrogen and C₁₋₆ alkyl;
each R⁵ is independently selected from the group consisting of hydrogen, C₁₋₆ alkyl, —C(═O)—C₁₋₆ alkyl, and —SO₂(R⁷);
each R⁶ is independently selected from the group consisting of hydrogen and C₁₋₆ alkyl;
R⁷ is selected from the group consisting of hydrogen, C₁₋₆ alkyl, and aryl optionally substituted with 1 to 3 of C₁₋₄ alkyl or halo;
R⁸ is selected from the group consisting of aryl, biaryl, heteroaryl, and heterocyclic, wherein each R⁸ is optionally substituted with 1-4 R⁹ groups;
each R⁹ is independently selected from the group consisting of hydroxy, halo, nitro, C₁₋₆ alkyl, C₂₋₆ alkenyl, C₁₋₆ alkoxy, C₁₋₆ haloalkoxy, C₃₋₁₀ cycloalkyl, aralkyl, aryl, —N(R⁵)(R⁶), carboxyl, carboxyl ester, and heterocyclic;
n is 0, 1, 2, or 3; and
m is 0 or 1;
or a pharmaceutically acceptable salt, stereoisomer, tautomer, or prodrug thereof;
with the proviso that the compound exhibits an IC₅₀ of about 50 μM or less in the FRET assay.

6. The method as in claim 3, wherein the disease is associated with formation of and/or activity of ADDLs.

7. The method as in claim 6, wherein the disease is selected from the group consisting of Alzheimer's disease, Down's Syndrome, stroke and mild cognitive impairment.

8. The method as in claim 3, wherein the disease associated with insoluble amyloid fibrils, senile plaques, and/or tangles.

9. The method as in claim 3, wherein the disease associated with over-expression of Aβ₁₋₄₂ protein.

10. The method as in claim 9, wherein disease is selected from the group consisting of focal ischemia associated dementia and neuronal degeneration.

11. A method of inhibiting, regulating and or modulating the binding of neurotoxic ADDLs to spines and/or synapses of a neuronal cell which comprises contacting said neuronal cell with an effective amount of a compound of the formula: 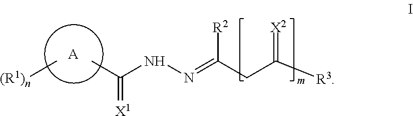
wherein:
A is a 5-10 membered heteroaryl ring having 1 to 3 heteroatoms or an aryl ring;
X¹ and X² are independently selected from the group consisting of oxygen, sulfur or N—OR⁴;
R¹ is selected from the group consisting of hydroxy, halo, nitro, C₁₋₆ alkyl, C₁₋₆ haloalkyl, C₁₋₆ alkoxy, —N(R⁵)(R⁶), C₃₋₁₀ cycloalkyl, aryl, heteroaryl, heterocyclic, wherein the aryl, heteroaryl, and heterocyclic group is optionally substituted with 1-3 R⁹ groups;
R² is selected from the group consisting of hydrogen, C₁₋₆ alkyl, and C₁₋₆ haloalkyl;
R³ is selected from the group consisting of C₁₋₆ alkyl, C₁₋₆ alkoxy, —N(R⁵)(R⁶), and R⁸;
R⁴ is selected from the group consisting of hydrogen and C₁₋₆ alkyl;
each R⁵ is independently selected from the group consisting of hydrogen, C₁₋₆ alkyl, —C(═O)—C₁₋₆ alkyl, and —SO₂(R⁷);
each R⁶ is independently selected from the group consisting of hydrogen and C₁₋₆ alkyl;
R⁷ is selected from the group consisting of hydrogen, C₁₋₆ alkyl, and aryl optionally substituted with 1 to 3 of C₁₋₄ alkyl or halo;
R⁸ is selected from the group consisting of aryl, biaryl, heteroaryl, and heterocyclic, wherein each R⁸ is optionally substituted with 1-4 R⁹ groups;
each R⁹ is independently selected from the group consisting of hydroxy, halo, nitro, C₁₋₆ alkyl, C₂₋₆ alkenyl, C₁₋₆ alkoxy, C₁₋₆ haloalkoxy, C₃₋₁₀ cycloalkyl, aralkyl, aryl, —N(R⁵)(R⁶), carboxyl, carboxyl ester, and heterocyclic;
n is 0, 1, 2, or 3; and
m is 0 or 1;
or a pharmaceutically acceptable salt, stereoisomer, tautomer, or prodrug thereof;
with the proviso that the compound exhibits an IC₅₀ of about 50 μM or less in the FRET assay.

12. A method of inhibiting, regulating and/or modulating the long term potentiation of neuronal cells which method comprises contacting said cells with an effective amount of a compound of the formula: 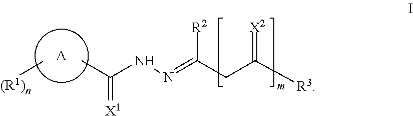
wherein:
A is a 5-10 membered heteroaryl ring having 1 to 3 heteroatoms or an aryl ring;
X¹ and X² are independently selected from the group consisting of oxygen, sulfur or N—OR⁴;
R¹ is selected from the group consisting of hydroxy, halo, nitro, C₁₋₆ alkyl, C₁₋₆ haloalkyl, C₁₋₆ alkoxy, —N(R⁵)(R⁶), C₃₋₁₀ cycloalkyl, aryl, heteroaryl, heterocyclic, wherein the aryl, heteroaryl, and heterocyclic group is optionally substituted with 1-3 R⁹ groups;
R² is selected from the group consisting of hydrogen, C₁₋₆ alkyl, and C₁₋₆ haloalkyl;
R³ is selected from the group consisting of C₁₋₆ alkyl, C₁₋₆ alkoxy, —N(R⁵)(R⁶), and R⁸;
R⁴ is selected from the group consisting of hydrogen and C₁₋₆ alkyl;
each R⁵ is independently selected from the group consisting of hydrogen, C₁₋₆ alkyl, —C(═O)—C₁₋₆ alkyl, and —SO₂(R⁷);
each R⁶ is independently selected from the group consisting of hydrogen and C₁₋₆ alkyl;
R⁷ is selected from the group consisting of hydrogen, C₁₋₆ alkyl, and aryl optionally substituted with 1 to 3 of C₁₋₄ alkyl or halo;
R⁸ is selected from the group consisting of aryl, biaryl, heteroaryl, and heterocyclic, wherein each R⁸ is optionally substituted with 1-4 R⁹ groups;
each R⁹ is independently selected from the group consisting of hydroxy, halo, nitro, C₁₋₆ alkyl, C₂₋₆ alkenyl, C₁₋₆ alkoxy, C₁₋₆ haloalkoxy, C₃₋₁₀ cycloalkyl, aralkyl, aryl, —N(R⁵)(R⁶), carboxyl, carboxyl ester, and heterocyclic;
n is 0, 1, 2, or 3; and
m is 0 or 1;
or a pharmaceutically acceptable salt, stereoisomer, tautomer, or prodrug thereof;
with the proviso that the compound exhibits an IC₅₀ of about 50 μM or less in the FRET assay.

13. A method of treating a patient suffering from diminished cognitive function due to suffering from or at risk of suffering from a disease selected from the group consisting of Alzheimer's disease, Down's Syndrome, stroke, mild cognitive impairment, focal ischemia associated dementia and neuronal degeneration, the method comprising administering to said patient a therapeutically effective amount of a compound of the formula: 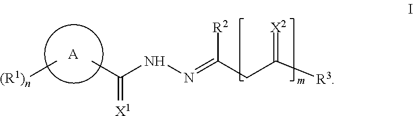
wherein:
A is a 5-10 membered heteroaryl ring having 1 to 3 heteroatoms or an aryl ring;
X¹ and X² are independently selected from the group consisting of oxygen, sulfur or N—OR⁴;
R¹ is selected from the group consisting of hydroxy, halo, nitro, C₁₋₆ alkyl, C₁₋₆ haloalkyl, C₁₋₆ alkoxy, —N(R⁵)(R⁶), C₃₋₁₀ cycloalkyl, aryl, heteroaryl, heterocyclic, wherein the aryl, heteroaryl, and heterocyclic group is optionally substituted with 1-3 R⁹ groups;
R² is selected from the group consisting of hydrogen, C₁₋₆ alkyl, and C₁₋₆ haloalkyl;
R³ is selected from the group consisting of C₁₋₆ alkyl, C₁₋₆ alkoxy, —N(R⁵)(R⁶), and R⁸;
R⁴ is selected from the group consisting of hydrogen and C₁₋₆ alkyl;
each R⁵ is independently selected from the group consisting of hydrogen, C₁₋₆ alkyl, —C(═O)—C₁₋₆ alkyl, and —SO₂(R⁷);
each R⁶ is independently selected from the group consisting of hydrogen and C₁₋₆ alkyl;
R⁷ is selected from the group consisting of hydrogen, C₁₋₆ alkyl, and aryl optionally substituted with 1 to 3 of C₁₋₄ alkyl or halo;
R⁸ is selected from the group consisting of aryl, biaryl, heteroaryl, and heterocyclic, wherein each R⁸ is optionally substituted with 1-4 R⁹ groups;
each R⁹ is independently selected from the group consisting of hydroxy, halo, nitro, C₁₋₆ alkyl, C₂₋₆ alkenyl, C₁₋₆ alkoxy, C₁₋₆ haloalkoxy, C₃₋₁₀ cycloalkyl, aralkyl, aryl, —N(R⁵)(R⁶), carboxyl, carboxyl ester, and heterocyclic;
n is 0, 1, 2, or 3; and
m is 0 or 1;
or a pharmaceutically acceptable salt, stereoisomer, tautomer, or prodrug thereof;
with the proviso that the compound exhibits an IC₅₀ of about 50 μM or less in the FRET assay.

14. The method as in claim 3, wherein the compound is administered in a pharmaceutical composition, further comprising a pharmaceutically acceptable excipient.

15. The method as in claim 14, wherein the compound is administered in an amount of from about 0.05 milligram to about 1000 milligram, one or more times per day.

16. The method as in claim 2, wherein said neuronal cell is isolated from animal brain tissue and grown in tissue culture.

17. The method as in claim 1 wherein the compound is of the formula: 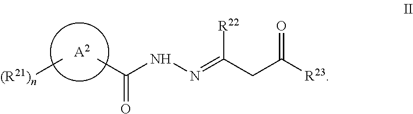
wherein:
A² is a 5-10 membered heteroaryl ring having 1 to 3 heteroatoms or an aryl ring;
R²¹ is selected from the group consisting of hydroxy, halo, nitro, C₁₋₆ alkyl, C₁₋₆ haloalkyl, —N—S(O)₂—R²⁴, and aryl;
R²² is selected from the group consisting of hydrogen, C₁₋₆ alkyl, and C₁₋₆ haloalkyl;
R²³ is selected from the group consisting of C₁₋₆ alkyl, amino, and R²⁵;
R²⁴ is selected from the group consisting of C₁₋₆ alkyl, and aryl optionally substituted with halo or C₁₋₆ alkyl,
R²⁵ is selected from the group consisting of aryl, heteroaryl, and heterocyclic, all of which may be optionally substituted with 1-3 R²⁶ groups;
each R²⁶ is independently selected from the group consisting of hydroxy, halo, C₁₋₆ alkyl, aralkyl, and aryl;
n is 0, 1, 2, or 3;
or a pharmaceutically acceptable salt, stereoisomer, tautomer, or prodrug thereof;
with the proviso that the compound exhibits an IC₅₀ of about 50 μM or less in the FRET assay.

18. The method as in claim 1 wherein the compound is of the formula: 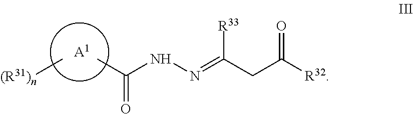
wherein:
A¹ is a 5-10 membered heteroaryl ring having 1 to 3 heteroatoms or an aryl ring;
R³¹ is selected from the group consisting of hydroxy, halo, nitro, C₁₋₆ alkyl, C₁₋₆ alkoxy, C₃₋₁₀ cycloalkyl, aryl, heteroaryl optionally substituted with 1-3 C₁₋₆ alkyl, amino, —N—SO₂—R³⁴, and —N—C(═O)—R³⁴;
R³² is selected from the group consisting of hydrogen, C₁₋₆ alkyl, and C₁₋₆ haloalkyl;
R³³ is selected from the group consisting of aryl, biaryl, heteroaryl, and heterocyclic, wherein each R³³ is optionally substituted with 1-4 R³⁵ groups;
R³⁴ is selected from the group consisting of C₁₋₆ alkyl and aryl optionally substituted with halo;
R³⁵ is selected from the group consisting of hydroxy, nitro, halo, C₁₋₆ alkyl, C₂₋₆ alkenyl, C₁₋₆ alkoxy, C₁₋₆ haloalkoxy, C₃₋₁₀ cycloalkyl, halo, amino, alkylamino, dialkylamino, aminoacyl, aryl-alkylene, carboxyl, carboxyl ester, and heterocyclic; and
n is 0, 1, 2, or 3;
or a pharmaceutically acceptable salt, stereoisomer, tautomer, or prodrug thereof.

19. The method as in claim 1, wherein the compound has an IC₅₀ of about 25 μM or less.

20. The method as in claim 1, wherein the compound has an IC₅₀ of about 10 μM or less.

21. The method as in claim 1, wherein the compound has an IX₅₀ of about 5 μM or less.

22. The method as in claim 1, wherein A is selected from the group consisting of phenyl, naphthyl, benzothiophenyl, thiadiazolyl, indanyl, thiophenyl, indolyl, pyrazolyl, furanyl, oxoindolinyl, pyridyl, and benzodioxoyl.

23. The method as in claim 1, wherein R¹ is selected from the group hydroxy, chloro, fluoro, bromo, iodo, methyl, methoxy, trifluoromethyl, cyclopropyl, phenyl, pyrrolyl, methylsulfonamido, 4-chlorophenylsulfonylamido, nitro, benzo[d][1,3]dioxolyl, amino, thienyl, 5-chlorothienyl, and methylcarbonylamino.

24. The method as in claim 1, wherein A is optionally substituted and is selected from the group consisting of 2-(methylsulfonamido)phenyl, 1H-indan-7-yl, 1H-indol-7-yl, 1-hydroxy-naphthalen-2-yl, 1-phenyl-5-(trifluoromethyl)-1H-pyrazol-4-yl, 2-(4-chlorophenyl sulfonamido) phenyl, 2,4-dihydroxyphenyl, 2,6-difluorophenyl, 2-acetamidophenyl, 2-aminophenyl, 2-benzothiophene, 2-fluoro-6-hydroxyphenyl, 2-hydroxy-3-methylphenyl, 2-hydroxy-4-(1H-pyrrol-1-yl)phenyl, 2-hydroxy-4-methoxyphenyl, 2-hydroxy-5-methoxyphenyl, 2-hydroxy-5-methylphenyl, 2-hydroxy-5-nitrophenyl, 2-hydroxy-naphthalen-1-yl, 2-hydroxyphenyl, 2-methylfuran-3-yl, 2-oxoindolin-7-yl, 3-(5-chlorothiophen-2-yl)-1H-pyrazol-5-yl, 3-(benzo[d][1,3]dioxol-5-yl-1H-pyrazol-5-yl, 3-aminophenyl, 3-chloro-1H-indol-2-yl, 3-chloro-4-methyl-thiophen-2-yl, 3-chloro-6-fluorobenzo thiophene, 3-chlorobenzothiophen-2-yl, 3-cyclopropyl-1H-pyrazol-5-yl, 3-fluorophenyl, 3-hydroxy-naphthalen-2-yl, 3-methyl-1H-pyrazol-5-yl, 4-(2,5-dimethyl-1H-pyrrol-1-yl)phenyl, 4-bromophenyl, 4-chloro-2-hydroxyphenyl, 4-iodo-1-methyl-1H-pyrazol-3-yl, 4-iodophenyl, 4-methyl-1,2,3-thiadiazol-5-yl, 4-methylphenyl, 4-nitrophenyl, 5-bromo-2-hydroxyphenyl, 6-methyl-pyrid-3-yl, 8-hydroxynaphthalen-1-yl, and benzo[d][1,3]dioxol-5-yl.

25. The method as in claim 21, wherein X¹ and X² are oxygen.

26. The method as in claim 22, wherein R² is selected from hydrogen, methyl, or trifluoromethyl.

27. The method as in claim 26 wherein m is 0.

28. The method as in claim 26 wherein m is 1.

29. The method as in claim 26, wherein R³ is selected from the group consisting of butyl, t-butyl, and amino.

30. The method as in claim 26 wherein R³ is optionally substituted and is selected from the group consisting of phenyl, biphenyl, thienyl, naphthyl, furanyl, piperazinyl, benzothiophenyl, pyrazolyl, morpholino, and piperidinyl.

31. The method as in claim 30 wherein R³ is substituted with one to four groups independently selected from the group consisting of hydroxy, chloro, fluoro, bromo, iodo, methyl, t-butyl, methoxy, ethoxy, benzyl, phenyl, cyclohexyl, trifluoromethoxy, allyl, aminocarbonyl, amino, ethoxycarbonyl, diethylamino, morpholino, nitro, 2,4-difluorophenylsulfonylamino, and methylcarbonylamino.

32. The method as in claim 26, wherein R³ is selected from the group consisting of 5-chloro-2-(2,4-difluorophenylsulfonamido)phenyl, 1-hydroxy-napthalen-2-yl, 1-methyl-1H-pyrazol-5-yl, 2,3-dihydroxyphenyl, 2,4-dihydroxyphenyl, 2,6-dihydroxyphenyl, 2-acetamido-5-chlorophenyl, 2-amino-5-chlorophenyl, benzothiophen-2-yl, 2-bromo-6-hydroxyphenyl, 2-furanyl, 2-hydroxy naphthalen-1-yl, 4-hydroxy-3′-methoxybiphen-3-yl, 2-hydroxy-3-methoxyphenyl, 2-hydroxy-3-methylphenyl, 2-hydroxy-4-methoxyphenyl, 2-hydroxy-4-methylphenyl, 2-hydroxy-4-morpholinophenyl, 2-hydroxy-5-methoxyphenyl, 2-hydroxy-5-methylphenyl, 2-hydroxy-5-nitrophenyl, 2-hydroxy-5-trifluoro methoxyphenyl, 2-hydroxy-6-methoxy-phenyl, 2-hydroxynaphthalen-1-yl, 2-hydroxyphenyl, 3,5-dibromo-2-hydroxyphenyl, 3,5-dichloro-2-hydroxy-phenyl, 3,5-difluoro-2-hydroxyphenyl, 3-allyl-2-hydroxyphenyl, 3-bromo-2-hydroxy-5-methoxyphenyl, 3-bromo-5-chloro-2-hydroxyphenyl, 3-chloro-5-cyclohexyl-2-hydroxyphenyl, 3-chloro-5-fluoro-2-hydroxyphenyl, 3-ethoxy-2-hydroxyphenyl, 3-fluoro-2-hydroxyphenyl, 3-hydroxy-5-nitrobenzofuran-2-yl, 4-benzyl piperazin-1-yl, 4-diethylamino-2-hydroxyphenyl, 4-methyl piperazin-1-yl, 4-methylphenyl, 4-morpholino, 4-phenylpiperidin-1-yl, 5-bromo-2-hydroxy-3-iodophenyl, 5-bromo-2-hydroxyphenyl, 5-chloro thiophen-2-yl, 2-amino-5-chloro-phenyl, 5-chloro-2-hydroxy-3-methoxyphenyl, 5-chloro-2-hydroxyphenyl, 5-chlorothiophen-2-yl, 5-ethoxy-2-hydroxyphenyl, 5-methyl thiophen-2-yl, 5-tert-butyl-2-hydroxyphenyl, 6-bromo-5-hydroxy-2-(ethoxycarbonyl)benzofuran-4-yl, benzamid-2-yl, —NH₂, tert-butyl, and thien-2-yl.

33. The method as in claim 1, wherein the compound is selected from the group consisting of the compounds in Table 1A and 1B.

34. A compound selected from the group consisting of:
2-hydroxy-N-(1,1,1-trifluoro-4-(furan-2-yl)-4-oxobutan-2-ylidene)benzohydrazide;
N-(4-(4-benzylpiperazin-1-yl)-1,1,1-trifluoro-4-oxobutan-2-ylidene)-2-hydroxybenzohydrazide;
3-chloro-6-fluoro-N′-(4-(furan-2-yl)-4-oxobutan-2-ylidene)benzo[b]thiophene-2-carbohydrazide;
N-(2-(2-(1,1,1-trifluoro-4-oxo-4-(thiophen-2-yl)butan-2-ylidene)hydrazinecarbonyl)phenyl)methanesulfonamide;
2-hydroxy-N′-(1,1,1-trifluoro-4-(4-methylpiperazin-1-yl)-4-oxobutan-2-ylidene)benzohydrazide;
2-hydroxy-N-(1,1,1-trifluoro-5,5-dimethyl-4-oxohexan-2-ylidene)benzohydrazide;
N-(4-(benzo[b]thiophen-2-yl)-1,1,1-trifluoro-4-oxobutan-2-ylidene)-2-hydroxybenzohydrazide;
2-hydroxy-N′-(1,1,1-trifluoro-4-(1-methyl-1H-pyrazol-5-yl)-4-oxobutan-2-ylidene)benzohydrazide;
3-chloro-6-fluoro-N-(1,1,1-trifluoro-4-(5-methylthiophen-2-yl)-4-oxobutan-2-ylidene)benzo[b]thiophene-2-carbohydrazide;
3-chloro-6-fluoro-N′-(1,1,1-trifluoro-5,5-dimethyl-4-oxohexan-2-ylidene)benzo[b]thiophene-2-carbohydrazide;
2-hydroxy-N′-(1,1,1-trifluoro-4-(5-methylthiophen-2-yl)-4-oxobutan-2-ylidene)benzohydrazide;
3-chloro-N-(1,1,1-trifluoro-4-oxo-4-(thiophen-2-yl)butan-2-ylidene)benzo[b]thiophene-2-carbohydrazide;
3-chloro-N-(1,1,1-trifluoro-4-(furan-2-yl)-4-oxobutan-2-ylidene)benzo[b]thiophene-2-carbohydrazide;
4-chloro-N-(2-(2-(1,1,1-trifluoro-4-oxo-4-(thiophen-2-yl)butan-2-ylidene)hydrazinecarbonyl)phenyl)benzenesulfonamide;
N′-(4-(benzo[b]thiophen-2-yl)-1,1,1-trifluoro-4-oxobutan-2-ylidene)-3-chloro-6-fluorobenzo[b]thiophene-2-carbohydrazide;
2-hydroxy-N-(1,1,1-trifluoro-4-morpholino-4-oxobutan-2-ylidene)benzohydrazide;
N′-(4-(5-chlorothiophen-2-yl)-1,1,1-trifluoro-4-oxobutan-2-ylidene)-2-hydroxybenzohydrazide;
5-chloro-2-hydroxy-N-(1,1,1-trifluoro-4-oxo-4-(thiophen-2-yl)butan-2-ylidene)benzohydrazide;
3-chloro-4-methyl-N-(1,1,1-trifluoro-4-oxo-4-(thiophen-2-yl)butan-2-ylidene)thiophene-2-carbohydrazide;
N′-(1,1,1-trifluoro-4-oxo-4-(thiophen-2-yl)butan-2-ylidene)-1H-indole-7-carbohydrazide;
3-chloro-N′-(4-(5-chlorothiophen-2-yl)-1,1,1-trifluoro-4-oxobutan-2-ylidene)-6-fluorobenzo[b]thiophene-2-carbohydrazide;
3-chloro-6-fluoro-N′-(1,1,1-trifluoro-4-oxo-4-(thiophen-2-yl)butan-2-ylidene)benzo[b]thiophene-2-carbohydrazide;
2-hydroxy-3-methyl-N′-(1,1,1-trifluoro-4-oxo-4-(thiophen-2-yl)butan-2-ylidene)benzohydrazide;
4-nitro-N′-(1,1,1-trifluoro-4-oxo-4-(thiophen-2-yl)butan-2-ylidene)benzohydrazide;
N′-(1,1,1-trifluoro-4-oxo-4-(thiophen-2-yl)butan-2-ylidene)benzo[b]thiophene-2-carbohydrazide;
2-hydroxy-N′-(4-oxo-4-(4-phenylpiperidin-1-yl)butan-2-ylidene)benzohydrazide;
3-chloro-N′-(1,1,1-trifluoro-4-oxo-4-(thiophen-2-yl)butan-2-ylidene)-1H-indole-2-carbohydrazide;
3-chloro-6-fluoro-N′-(1,1,1-trifluoro-4-(furan-2-yl)-4-oxobutan-2-ylidene)benzo[b]thiophene-2-carbohydrazide;
3-(2-(2-hydroxybenzoyl)hydrazono)butanamide;
1-phenyl-N′-(1,1,1-trifluoro-4-oxo-4-(thiophen-2-yl)butan-2-ylidene)-5-(trifluoromethyl)-1H-pyrazole-4-carbohydrazide;
2,6-difluoro-N′-(1,1,1-trifluoro-4-oxo-4-(thiophen-2-yl)butan-2-ylidene)benzohydrazide;
2-hydroxy-N′-(2-hydroxy-5-methoxybenzylidene)-4-(1H-pyrrol-1-yl)benzohydrazide;
1-hydroxy-N′-(2-hydroxy-5-methoxybenzylidene)-2-naphthohydrazide;
N′-(3-chloro-5-fluoro-2-hydroxybenzylidene)-2-hydroxybenzohydrazide;
N′-(2,6-dihydroxybenzylidene)-2-hydroxybenzohydrazide;
3-chloro-N′-(2-hydroxy-5-methoxybenzylidene)-1H-indole-2-carbohydrazide;
N′-(2-bromo-6-hydroxybenzylidene)-2-hydroxybenzohydrazide;
N′-(3-chloro-5-cyclohexyl-2-hydroxybenzylidene)-2-hydroxybenzohydrazide;
N′-(5-tert-butyl-2-hydroxybenzylidene)-2-hydroxybenzohydrazide;
2-hydroxy-N′-((4-hydroxy-3′-methoxybiphenyl-3-yl)methylene)benzohydrazide;
4-chloro-2-hydroxy-N′-(2-hydroxy-5-methoxybenzylidene)benzohydrazide;
N′-(2-hydroxy-5-methoxybenzylidene)-2-oxoindoline-7-carbohydrazide;
2-hydroxy-N′-(2-hydroxy-6-methoxybenzylidene)benzohydrazide;
2-hydroxy-N′-(2-hydroxy-5-(trifluoromethoxy)benzylidene)benzohydrazide;
N′-(2-hydroxy-5-methoxybenzylidene)-1H-indole-7-carbohydrazide;
3-chloro-6-fluoro-N′-((2-hydroxynaphthalen-1-yl)methylene)benzo[b]thiophene-2-carbohydrazide;
2-hydroxy-N′-(2-hydroxy-5-methoxybenzylidene)-3-methylbenzohydrazide;
2-((2-(2-hydroxybenzoyl)hydrazono)methyl)benzamide;
N′-(2-amino-5-chlorobenzylidene)-2-hydroxybenzohydrazide;
2-hydroxy-N′-(2-hydroxy-5-methoxybenzylidene)-1-naphthohydrazide;
4-fluoro-2-hydroxy-N′-(2-hydroxy-5-methoxybenzylidene)benzohydrazide;
3-chloro-N′-(2-hydroxy-5-methoxybenzylidene)-4-methylthiophene-2-carbohydrazide;
2-hydroxy-N′-(2-hydroxy-5-methoxybenzylidene)-5-methylbenzohydrazide;
N′-(3-fluoro-2-hydroxybenzylidene)-2-hydroxybenzohydrazide;
5-fluoro-2-hydroxy-N′-(2-hydroxy-5-methoxybenzylidene)benzohydrazide;
ethyl 6-bromo-5-hydroxy-4-((2-(2-hydroxybenzoyl)hydrazono)methyl)-2-methylbenzofuran-3-carboxylate;
3-(benzo[d][1,3]dioxol-5-yl)-N′-(2-hydroxy-5-methoxybenzylidene)-1H-pyrazole-5-carbohydrazide;
N′-(4-(diethylamino)-2-hydroxybenzylidene)-2-hydroxybenzohydrazide;
2-hydroxy-N′-(2-hydroxy-4-methoxybenzylidene)benzohydrazide;
2-hydroxy-N′-(2-hydroxy-5-methoxybenzylidene)-5-methoxybenzohydrazide;
2-hydroxy-N′-(2-hydroxy-5-methoxybenzylidene)benzohydrazide;
N′-(5-ethoxy-2-hydroxybenzylidene)-2-hydroxybenzohydrazide;
N′-(2,3-dihydroxybenzylidene)-2-hydroxybenzohydrazide;
2-hydroxy-N′-(2-hydroxy-4-morpholinobenzylidene)benzohydrazide;
2-hydroxy-N′-((3-hydroxy-5-nitrobenzofuran-2-yl)methylene)benzohydrazide;
N′-(2,4-dihydroxybenzylidene)-2-hydroxybenzohydrazide;
N′-(5-chloro-2-hydroxy-3-methoxybenzylidene)-2-hydroxybenzohydrazide;
3-chloro-N′-(5-chloro-2-hydroxybenzylidene)-4-methylthiophene-2-carbohydrazide;
2-amino-N′-(2-amino-5-chlorobenzylidene)benzohydrazide;
2-hydroxy-N′-(2-hydroxy-3-methoxybenzylidene)benzohydrazide;
2-fluoro-6-hydroxy-N′-(2-hydroxy-5-methoxybenzylidene)benzohydrazide;
2-hydroxy-N′-(2-hydroxy-3-methylbenzylidene)benzohydrazide;
N′-((2-hydroxynaphthalen-1-yl)methylene)-3-methyl-1H-pyrazole-5-carbohydrazide;
5-bromo-2-hydroxy-N′-(2-hydroxy-5-methoxybenzylidene)benzohydrazide;
2-hydroxy-N′-(2-hydroxy-5-nitrobenzylidene)benzohydrazide;
N-(2-(2-(2-hydroxy-5-methoxybenzylidene)hydrazinecarbonyl)phenyl)methanesulfonamide;
N′-(3,5-difluoro-2-hydroxybenzylidene)-2-hydroxybenzohydrazide;
N′-(1-(5-chloro-2-hydroxyphenyl)-2,2,2-trifluoroethylidene)-2-hydroxybenzohydrazide;
2-hydroxy-N′-(2-hydroxy-5-methoxybenzylidene)-5-nitrobenzohydrazide;
8-hydroxy-N′-(2-hydroxy-5-methoxybenzylidene)-1-naphthohydrazide;
N′-(3-ethoxy-2-hydroxybenzylidene)-2-hydroxybenzohydrazide;
3-(5-chlorothiophen-2-yl)-N′-(2-hydroxy-5-methoxybenzylidene)-1H-pyrazole-5-carbohydrazide;
N′-(3-bromo-5-chloro-2-hydroxybenzylidene)-2-hydroxybenzohydrazide;
N′-(3-bromo-2-hydroxy-5-methoxybenzylidene)-2-hydroxybenzohydrazide;
2-amino-N′-(2-hydroxy-5-methoxybenzylidene)benzohydrazide;
N′-(5-chloro-2-hydroxybenzylidene)-4-methyl-1,2,3-thiadiazole-5-carbohydrazide;
N-(2-(2-(2-acetamido-5-chlorobenzylidene)hydrazinecarbonyl)phenyl)acetamide; and
N-(4-chloro-2-((2-(2-hydroxybenzoyl)hydrazono)methyl)phenyl)-2,4-difluorobenzenesulfonamide;
or a pharmaceutically acceptable salt, stereoisomer, tautomer, or prodrug thereof.
